SEQUENCE LISTING (40 sequences)

SEQ ID NO: 1 - protein, 465 aa, Myceliophthora thermophila
APVIEERQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGATSTASYSGNPFSGVRLFANDYYRSEVHNLAIPSMTGTLAAKASAVAEVPSFQWLDRNVTIDTLMVQTLSQVRALNKAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPPF

SEQ ID NO: 2 - protein, 465 aa, Artificial sequence
  Synthetic polypeptide of Myceliophthora thermophila cellobiohydrolase type 2b variant 81 without signal peptide
APVIEERQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGATSTASYSGNPFSGVRLFANDYYRSEVHNLAIPSMTGTLAAKASAVAEVPSFQWLDRNVTIDTLMVQTLSQVRALNKAGANPPYAAQLVVYDLPDRDCAAAASNGEFSIANGGAANYRSYIDAIRKHIIEYPDIRIILVIEPDSMANMVTNMNVPKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNSAGFPARFIVDTGRNGKQPTGQQQWGDWCNVKGTGFGVRPTANTGHPLVDAFVWVKPGGESDGTSDTSAARYDYHCGLPDALQPAPEAGQWFQAYFEQLLTNANPPF

SEQ ID NO: 3 - protein, 465 aa, Artificial sequence
  Synthetic polypeptide of Myceliophthora thermophila cellobiohydrolase type 2b variant 160 without signal peptide
APVIEESQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGAGSTASYSGNPFSGVRLFANDHYRSEVHNLAIPSMTGTLAAKASAVAEVPSFQWLDRNVTIDTLMVRTLSQVRALNKAGANPPYAAQLVVYDLPDRDCAAAASNGEFSIANGGAANYRSYIDAIRKHIIEYPDIRIILVIEPDSMANMVTNMNVPKCSNAASTYHELTVYALKQLNLPNVAMYLDAGHAGWLGWPANIQPAAELFAGIYNDAGKPAAVRGLATNVANYNAWSIASAPSYTQPNPNYDEKHYIEAFSPLLNSAGFPARFIVDTGRNGKQPTGQQQWGDWCNVKGTGFGVRPTANTGHPLVDAFVWVKPGGESDGTSDTSAARYDYHCGLPDALQPAPEAGQWFQAYFEQLLNNANPPF

SEQ ID NO: 4 - protein, 465 aa, Artificial sequence
  Synthetic polypeptide of Myceliophthora thermophila cellobiohydrolase type 2b variant 155 without signal peptide
APVIEESQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGAGSTASYSGNPFSGVRLFANDHYRSEVHNLAIPSMTGTLAAKASAVAEVPSFQWLDRNVTIDTLMVRTLSQVRALNKAGANPPYAAQLVVYDLPDRDCAAAASNGEFSIANGGAANYRSYIDAIRKHIMEYPDIRIILVIEPDSMANMVTNMNVPKCSNAASTYHELTVYALKQLNLPNVAMYLDAGHAGWLGWPANIQPAAELFAGIYNDAGKPAAVRGLATNVANYNAWSIASAPSYTQPNPNYDEKHYIEAFSPLLNSAGFPARFIVDTGRNGKQPTGQQQWGDWCNVKGTGFGVRPTANTGHPLVDAFVWVKPGGESDGTSDTSAARYDYHCGLPDALQPAPEAGQWFQAYFEQLLNNANPPF

SEQ ID NO: 5 - protein, 362 aa, Humicola insolens
YNGNPFEGVQLWANNYYRSEVHTLAIPQITDPALRAAASAVAEVPSFQWLDRNVTVDTLLVQTLSEIREANQAGANPQYAAQIVVYDLPDRDCAAAASNGEWAIANNGVNNYKAYINRIREILISFSDVRTILVIEPDSLANMVTNMNVPKCSGAASTYRELTIYALKQLDLPHVAMYMDAGHAGWLGWPANIQPAAELFAKIYEDAGKPRAVRGLATNVANYNAWSVSSPPPYTSPNPNYDEKHYIEAFRPLLEARGFPAQFIVDQGRSGKQPTGQKEWGHWCNAIGTGFGMRPTANTGHQYVDAFVWVKPGGECDGTSDTTAARYDYHCGLEDALKPAPEAGQWFNEYFIQLLRNANPPF

SEQ ID NO: 6 - protein, 459 aa, Chaetomium thermophilum
APLLEERQSCSSVWGQCGGINYNGPTCCQSGSVCAYLNDWYSQCIPGQAQPGTTSTTARTTSTSTTSTSSVRPTTSNTPVTTAPPTTTIPGGASSTASYNGNPFSGVQLWANTYYSSEVHTLAIPSLSPELAAKAAKVAEVPSFQWLDRNVTVDTLFSGTLAEIRAANQRGANPPYAGIFVVYDLPDRDCAAAASNGEWSIANNGANNLQRYIDRIRELLIQYSDIRTILVIEPDSLANMVTNMNVQKCSNAASTYKELTVYALKQLNLPHVAMYMDAGHAGWLGWPANIQPAAELFAQIYRDAGRPAAVRGLATNVANYNAWSIASPPSYTSPNPNYDEKHYIEAFAPLLRNQGFDAKFIVDTGRNGKQPTGQLEWGHWCNVKGTGFGVRPTANTGHELVDAFVWVKPGGESDGTSDTSAARYDYHCGLSDALTPAPEAGQWFQAYFEQLLINANPPF

SEQ ID NO: 7 - protein, 460 aa, Humicola insolens
APVVEERQNCAPTWGQCGGIGFNGPTCCQSGSTCVKQNDWYSQCLPGSQVTTTSTTSTSSSSTTSRATSTTRTGGVTSITTAPTRTVTIPGGATTTASYNGNPFEGVQLWANNYYRSEVHTLAIPQITDPALRAAASAVAEVPSFQWLDRNVTVDTLLVETLSEIRAANQAGANPPYAAQIVVYDLPDRDCAAAASNGEWAIANNGANNYKGYINRIREILISFSDVRTILVIEPDSLANMVTNMNVAKCSGAASTYRELTIYALKQLDLPHVAMYMDAGHAGWLGWPANIQPAAELFAKIYEDAGKPRAVRGLATNVANYNAWSISSPPPYTSPNPNYDEKHYIEAFRPLLEARGFPAQFIVDQGRSGKQPTGQKEWGHWCNAIGTGFGMRPTANTGHQYVDAFVWVKPGGECDGTSDTTAARYDYHCGLEDALKPAPEAGQWFQAYFEQLLRNANPPF

SEQ ID NO: 8 - protein, 468 aa, Artificial sequence
  Synthetic polypeptide of hypothetical protein CHGG-10762 from Chaetomium globosum CBS 148.51
APVVEERQNCATLWGQCGGNGWNGATCCASGSTCTKQNDWYSQCLPGGAVTTPGTTTKPTSTSTSTSTSSRSTSTSQGGGVSSSTSSPPVVTNPPTSIPGGASSTASYTGNPFSGVQMWANDYYRSEVHTLAMPSLTGAMATKAAKVAEVPSYQWMDRNVTVDTLFSGTLAQIRAANQAGASPPYAGIFVVYDLPDRDCAAAASNGEWSIANGGAANYKAYIKRIRELIIQYSDIRMLLVIEPDSLANMVTNMGVAKCAGAASTYKELTIHALKELNLPNVAMYLDAGHAGWLGWPANIQPAADLFATLYKDAGRPAAVRGLATNVANYNAWSVSSAPAYTSPNPNYDEKHYVEAFSPLLTAAGFPAHFITDTGRSGKQPTGQLEWGHWCNAVGTGFGQRPSANTGHDLLDAFVWIKPGGECDGTSDTTAARYDHNCGLADALKPAPEAGQWFQAYFEQLLTNANPPF

SEQ ID NO: 9 - protein, 453 aa, Humicola insolens
ASCAPTWGQCGGIGFNGPTCCQSGSTCVKQNDWYSQCLPGSQVTTTSTTSTSSSSTTSRATSTTSTGGVTSITTAPTRTVTIPGGATTTASYNGNPFEGVQLWANNYYRSEVHTLAIPQITDPALRAAASAVAEVPSFQWLDRNVTVDTLLVETLSEIRAANQAGANPPYAAQIVVYDLPDRDCAAAASNGEWAIANNGANNYKGYINRIREILISFSDVRTILVIEPDSLANMVTNMNVAKCSGAASTYRELTIYALKQLDLPHVAMYMDAGHAGWLGWPANIQPAAELFAKIYEDAGKPRAVRGLATNVANYNAWSISSPPPYTSPNPNYDEKHYIEAFRPLLEARGFPAQFIVDQGRSGKQPTGQKEWGHWCNAIGTGFGMRPTANTGHQYVDAFVWVKPGGECDGTSDTTAARYDYHCGLEDALKPAPEAGQWFQAYFEQLLRNANPPF

SEQ ID NO: 10 - protein, 467 aa, Artificial sequence
  Synthetic polypeptide of hypothetical protein from Podospora anserina S mat+
APVIEERQNCGSVWSQCGGQGWTGATCCASGSTCVAQNQWYSQCLPGSQVTTTAQAPSSTRTTTSSSSRPTSSSISTSAVNVPTTTTSAGASVTVPPGGGASSTASYSGNPFLGVQQWANSYYSSEVHTLAIPSLTGPMATKAAAVAKVPSFQWMDRNVTVDTLFSGTLADIRAANRAGANPPYAGIFVVYDLPDRDCAAAASNGEWAIADGGAAKYKAYIDRIRHHLVQYSDIRTILVIEPDSLANMVTNMNVPKCQGAANTYKELTVYALKQLNLPNVAMYLDAGHAGWLGWPANIGPAAELFAGIYKDAGRPTSLRGLATNVANYNGWSLSSAPSYTTPNPNFDEKRFVQAFSPLLTAAGFPAHFITDTGRSGKQPTGQLEWGHWCNAIGTGFGPRPTTDTGLDIEDAFVWIKPGGECDGTSDTTAARYDHHCGFADALKPAPEAGQWFQAYFEQLLTNANPPF

SEQ ID NO: 11 - protein, 463 aa, Sordaria macrospora
APVLEDRQNCGSSWSQCGGIGWSGATCCSSGNYCSEINPYYFQCLPGAATTTKASSTSPTSTSKVSSTTSKVTTSSANQPITTTAPSVPTTTIAGGASSTASFTGNPFVGVQGWANSYYSSEIYNHAIPSMTGTWAAKASAVAKVPTFQWLDRNITVDTLMKSTLQEIRAANKAGANPPYAAHFVVYDLPDRDCAAAASNGEFSLANNGINNYKTYINAIRKLLVEYSDIRTILVVEPDSLANLVTNTNVAKCANAASAYKECTNYAITQLDLPHVAQYLDAGHGGWLGWPANIGPAATLFADVYKNAGKPKSVRGLVTNVSNYNGWSLASAPSYTTPNPNYDEKRFVEAFSPLLNAAGFPAQFIVDTGRSGMQPTGQIEQGDWCNAIGTGFGTRPTTNTGSSITDAFVWVKPGGESDGTSNTSAARYDFHCGLSDALKPAPEAGQWFQAYFEQLLKNANPAF

SEQ ID NO: 12 - protein, 438 aa, Artificial sequence
  Synthetic polypeptide of hypothetical protein BC1G_08989 from Botryotinia fuckeliana B05.10
QGAAYAQCGGQGWSGATTCVSGYTCVVNNAYYSQCLPGSAVTTTATTAPTATTPTTIITSTTKATTTTGGSSATTTAAVAGNPFSGKALYANPYYASEISASAIPSLTGAMATKAAAVAKVPTFYWLDTAAKVPLMGTYLANIRALNKAGANPPVAGTFVVYDLPDRDCAAAASNGEYSIADGGLVKYKAYIDSIVALLKTYSDVSVILVIEPDSLANLVTNLSVAKCSNAQAAYLEGTEYAIAQLNLPNVAMYLDAGHAGWLGWPANIGPAAQLFGQIYKAAGSPAAVRGLATNVANYNAWTSTTCPSYTSGDSNCNEKLYINALAPLLTAQGFPAHFIMDTSRNGVQPTAQQAWGDWCNLIGTGFGVRPTTNTGDALEDAFVWIKPGGEGDGTSDTTAARYDFHCGLADALKPAPEAGTWFQAYFAQLLTNANPSF

SEQ ID NO: 13 - protein, 450 aa, Artificial sequence
  Synthetic polypeptide of hypothetical protein NECHADRAFT_73991 from Nectria haematococca mpVI 77-13-4
APLVEERQACAAQWAQCGGFSWNGATCCQSGSYCSKINDYYSQCIPGEGPATSKSSTLPASTTTTQPTSTSTAGTSSTTKPPPAGSGTATYSGNPYSGVNLWANSYYRSEVTNLAIPKLSGAMATAAAKVADVPSYQWMDSFDHISLMEDTLVDIRKANLAGGNYAGQFVVYDLPDRDCAAAASNGEYSLDNDGANKYKNYIQTIKKIIQSYSDIRILLVIEPDSLANLVTNMDVAKCAKAHDAYISLTNYAVTELNLPNVAMYLDAGHAGWLGWPANQGPAAKLFASIYKDAGKPAALRGLATNVANYNAWSLSSAPPYTQGASIYDEKSFIHAMGPLLEQNGWPGAHFITDQGRSGKQPTGQIQWGDWCNSKGTGFGIRPSANTGDSLLDAFVWVKPGGESDGTSDTSATRYDYHCGASAALQPAPEAGTWFQAYFEQLLTNANPSFL

SEQ ID NO: 14 - protein, 435 aa, Aspergillus fumigatus Af293
QQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 15 - protein, 447 aa, Trichoderma reesei [sequence omitted]

SEQ ID NO: 16 - protein, 439 aa, Gibberella zeae
APVEERQSCSNGVWSQCGGQNWSGTPCCTSGNKCVKVNDFYSQCQPGSADPSPTSTIVSATTTKATTTGSGGSVTSPPPVATNNPFSGVDLWANNYYRSEVSTLAIPKLSGAMATAAAKVADVPSFQWMDTYDHISFMEDSLADIRKANKAGGNYAGQFVVYDLPDRDCAAAASNGEYSLDKDGKNKYKAYIADQGILQDYSDTRIILVIEPDSLANMVTNMNVPKCANAASAYKELTIHALKELNLPNVSMYIDAGHGGWLGWPANLPPAAQLYGQLYKDAGKPSRLRGLVTNVSNYNAWKLSSKPDYTESNPNYDEQKYIHALSPLLEQEGWPGAKFIVDQGRSGKQPTGQKAWGDWCNAPGTGFGLRPSANTGDALVDAFVWVKPGGESDGTSDTSAARYDYHCGIDGAVKPAPEAGTWFQAYFEQLLKNANPSFL

SEQ ID NO: 17 - protein, 470 aa, Artificial sequence
  Synthetic polypeptide of hypothetical protein MGG_05520 from Magnaporthe oryzae 70-15
SPLAVEERQACAAQWGQCGGQDYTGPTCCQSGSTCVVSNQWYSQCLPGSSNPTTTSRTSTSSSSSTSRTSSSTSRPPSSVPTTPTSVPPTITTTPTTTPTGGSGPGTTASFTGNPFAGVNLFPNKFYSSEVHTLAIPSLTGSLVAKASAVAQVPSFQWLDIAAKVETLMPGALADVRAANAAGGNYAAQLVVYDLPDRDCAAAASNGEFSIADGGVVKYKAYIDAIRKQLLAYSDVRTILVIEPDSLANMVTNMGVPKCAGAKDAYLECTIYAVKQLNLPHVAMYLDGGHAGWLGWPANLQPAADLFGKLYADAGKPSQLRGMATNVANYNAWDLTTAPSYTTPNPNFDEKKYISAFAPLLAAKGWSAHFIIDQGRSGKQPTGQKEWGHWCNQQGVGFGRRPSANTGSELADAFVWIKPGGECDGVSDPTAPRFDHFCGTDYGAMSDAPQAGQWFQKYFEMLLTNANPPL

SEQ ID NO: 18 - protein, 381 aa, Pyrenophora tritici-repentis Pt-1C-BFP
LPQATGTPKPTGTSPSMTTAAASGNPFAGYNFYANPYYSSEVYTLAMPSLAASLKPAATAVANIGSFVWMDTMAKVPLMDTYLANIKAKNAAGAKLMGTFVVYDLPDRDCAALASNGELKISEGGAEKYKKQYIDKIAAIIQKYPDVKINLAIEPDSLANMVTNLGVAKCANAAPYYKDLTAYAISKLNFANVDMYLDGGHAGWLGWDANIGPAAKLYADVYKAAGKPRAVRGIVTNVSNYNAFRIATCPAITQGNKNCDEERYINAFAPLLQAEGFPAHFIVDTGRSGKQPTGQQAWGDWCNVSGAGFGARPSTNTGNANVDAFVWVKPGGESDGTSDQSAARYDSHCGVSSALKPAPEAGTWFQAYFEMLLKNASPALA

SEQ ID NO: 19 - protein, 446 aa, Verticillium albo-atrum VaMs.102
APLEERQACASQWGQCGGQGWSGPTCCPSGTTCQLQNAWYSQCLPGAAPPPAVTTTRPATTAASSTRPATTSSIRSTTVVNPPTTTVAPPPGTTVAPPPGTTVAPPPGGATYTGNPFAGVNQWANAYYRSEVSSLAVPSLSGPLATAAAKVADVPTFQWMDTTAKVPLIDGALADIRRANAAGGNYAGIFVVYNLPDRDCAAAASNGELSIANDGINKYKAYIDSIRTVLLKYNDIRTLLVIEPDSLANMVTNMGVAKCSNAAAAYKECTKYAVQKLDLPHVAQYLDAGHAGWLGWPANIGPAATIFTDIYKEAGKPKSLRGLATNVSNYNAWNASSPAPYTSPNPNYDEKHYVDAFAPLLRQNGWDAKFIIDQGRSGKQPTGQQEWGHWCNALGTGFGLRPTSNTGHPDVDAFVWVKPGGEADGTSDTTAVRYDHFCGSASSMKP

SEQ ID NO: 20 - protein, 429 aa, Artificial sequence
  Synthetic polypeptide of hypothetical protein SNOG_06409 from Phaeosphaeria nodorum SN15
SLYQQCGGTGFSGSTTCVSGAYCSKVNDSATSAAPAPTTFKTSKTVGSPATGSSTTGSSATGTASPGDGSNPLKGKNFYANSYYASEINNLAAPSLVAAGNAALAAKASNVAKVGTFYWLDVRAKVPIISTFAKDVQKRNAAGANEVLPLVVYDLPERDCAALASNGELSLANNGTALYQEYIDMIAAQIKQFPDVTFLLVVEPDSLANLVTNLNVAKCANAATAYKTLTAYAIKTLNLKNVIMYLDAGHAGWLGWTANIEPAAELFGALYKSAGSPAAVRGLVTNVANYNAWSIATCPSYTQGNTNCDEKRYVNALAPLLVKNGFPAHFLTDTGRNGVQPTKQQAWGDWCNVIGTGFGIRPSSTTDDPLLDAYVWVKPGGEGDGTSDTSAVRYDAHCGYADALKPAPEAGSWFQAYFVQLLSNASPAF

SEQ ID NO: 21 - protein, 418 aa, Agaricus bisporus
QSPVWGQCGGNGWTGPTTCASGSTCVKQNDFYSQCLPNNQAPPSTTTQPGTTPPATTTSGGTGPTSGAGNPYTGKTVWLSPFYADEVAQAAADISNPSLATKAASVAKIPTFVWFDTVAKVPDLGGYLADARSKNQLVQIVVYDLPDRDCAALASNGEFSLANDGLNKYKNYVDQIAAQIKQFPDVSVVAVIEPDSLANLVTNLNVQKCANAQSAYKEGVIYAVQKLNAVGVTMYIDAGHAGWLGWPANLSPAAQLFAQIYRDAGSPRNLRGIATNVANFNALRASSPDPITQGNSNYDEIHYIEALAPMLSNAGFPAHFIVDQGRSGVQNIRDQWGDWCNVKGAGFGQRPTTNTGSSLIDAIVWVKPGGECDGTSDNSSPRFDSHCSLSDAHQPAPEAGTWFQAYFETLVANANPAL

SEQ ID NO: 22 - protein, 422 aa, Volvariella volvacea
QSPLYGQCGGNGWTGPKTCVSGATCTVINDWYWQCLPGNGPTSSSPTSTPTTTTTTGGPQPTVPAAGNPYTGYEIYLSPYYAAEAQAAAAQISDATQKAKALKVAQIPTFTWFDVIAKTSTLGDYLAEASALGKSSGKKYLVQIVVYDLPDRDCAALASNGEFSIANNGLNNYKGYIDQLVAQIKKYPDVRVVAVIEPDSLANLVTNLNVSKCANAQTAYKAGVTYALQQLNSVGVYMYLDAGHAGWLGWPANLNPAAQLFSQLYRDAGSPQYVRGLATNVANYNALSASSPDPVTQGNPNYDELHYINALAPALQSGGFPAHFIVDQGRSGVQNIRQQWGDWCNVKGAGFGQRPTLSTGSSLIDAIVWIKPGGECDGTTNTSSPRYDSHCGLSDATPNAPEAGQWFQAYFETLVRNASPPL

SEQ ID NO: 23 - protein, 369 aa, Coniophora puteana
MPASTQARAADATANPYTGYTIFKNPEYVAEVQAAVQQISDSSLASAAAGVEDVPVFFWLDQVAKVPNLTTYLAAADAEAKSSGSQQLFQIVVYDLPDRDCAAAASNGEFSISDNGQANYENYIDQIVASIKQYPDVRVVAVVEPDSMANLVTNLSVQKCADAESTYKTCVAYAIEQLATVGVYMYLDAGHAGWLGWPANLSPAAELFAQMYSTTGSSPYFRGLATNVANYNSLTTDSPDPITSGDSNYDELLYIEALSPLLVDNGFPAQFIVEQARSGVQNIRSAWGDWCNVKGAGFGLRPSTDTPSSLIDSIVWVKPGGEADGTSNSSAARYDYHCSLSDALQPAPEAGTWFQTYFEDLVSGANPAF

SEQ ID NO: 24 - protein, 440 aa, Phaenerochaete chrysosporium
ASSEWGQCGGIGWTGPTTCVSGTTCTVLNPYYSQCLPGSAVTTTSVITSHSSSVSSVSSHSGSSTSTSSPTGPTGTNPPPPPSANNPWTGFQIFLSPYYANEVAAAAKQITDPTLSSKAASVANIPTFTWLDSVAKIPDLGTYLASASALGKSTGTKQLVQIVIYDLPDRDCAAKASNGEFSIANNGQANYENYIDQIVAQIQQFPDVRVVAVIEPDSLANLVTNLNVQKCANAKTTYLACVNYALTNLAKVGVYMYMDAGHAGWLGWPANLSPAAQLFTQVWQNAGKSPFIKGLATNVANYNALQAASPDPITQGNPNYDEIHYINALAPLLQQAGWDATFIVDQGRSGVQNIRQQWGDWCNIKGAGFGTRPTTNTGSQFIDSIVWVKPGGECDGTSNSSSPRYDSTCSLPDAAQPAPEAGTWFQAYFQTLVSAANPPL

SEQ ID NO: 25 - protein, 445 aa, Lentinus sajor-caju
VGEWGQCGGINYTGSTTCDAGLVCNVINDYYHQCLPTPDAGNPYIGYDVSHVLWCQIYLSPYYADEVAAAVSAISNPALAAKAASVANIPTFIWFDVVAKVPTLGTYLADALSIQQSTGRNQLVQIVVYDLPDRDCAALASNGEFSIANNGLANYKNYVDQIVAQIARTCCPLVTSAITDLACLSEYPQIRVVAVVEPDSLANMVTNLNVPKCAGAQAAYTEGVTYALQKLNTVGVYSYVDAGHAGWLGWPANLGPAAQLFANLYTNAGSPSFFRGLATNVANYNLLNAPSPDPVTSPNANYDEIHYINVSDCFVLIWTSLTICIIALAPELSSRGFPAHFIVDQGRSAVQGIRGAWGDWCNVDNAGFGTRPTTSTGSSLIDAIVWVKPGGESDGTSDTSAVRYDGHCGLASAKKPAPEAMASVYSHSSFQAYFEMLVANAVPAL

SEQ ID NO: 26 - protein, 440 aa, Coniophora puteana
QVAAYGQCGGQDWTGATACASGTACTKVNDYYYQCLPGSSGSSVSGGSGSGSTSAPSPTSTVPTSTSSASTAPSSTSTSSAASSDNPYTGYQIFLNPEYASEVQAAIPSITDSAVAAKALKVAEVPVFFWLDQVAKVPDLETYLAAADKQGKSSGQKQLLQIVVYDLPDRDCAANASNGEFSISDDGQAKYENYIDQIVAIVKKYPDVRVVAVVEPDSMGNLVTNMDLPKCSAAAPTYKTCINYAIAQLSSAGVYMYVDAGHAGWLGWPNNLAPAAQLFGELYETSGKSAYFRGLATNVANYNALNTSSPDPCTQNAPNYDEMLYINALSPLLQQQGFSAQFIVDQGRSGVQNIRNAWGDWCNIKGAGFGIRPTTDTGSPLIDSIVWVKPGGECDGTSNSSAPRYDSTCSLSDSLQPAPEAGTWFQQYFEALVTNAVPSL

SEQ ID NO: 27 - protein, 377 aa, Verticillium albo-atrum VaMs.102
VPHRNKCRDVATYEGNPLADVQLYPDPYYVNEIETLAIPQIEDEELVAAAKAVTKISTFQWLTSDKISKLDLLNSTLHEIRAANDAGASPPYAATIVVYNFPDRDCSAKASAGELILAEDGLNRYKTEYIDPIAALIKKFSDIRTVIAYEPDGLANLVTNMAVEKCANAASAYREATEYGLATLNFANVAIYVDAGHAGWLGWDGNLQPTAELYAELYKNAGSPKAVRGVVTNVSNFNGYNLTTPPAYTEPNAQWDESKFHDALAPHLETAGYPAHFIVDQGRSGVQPGLRSAWSHWCNINGTGFGPRPTTEIADEITDAIVWIKPGGESDGTSDETAVRFDENCHSPSAFQPAPEAGGWFQAYFEMLLKNANPPLA

SEQ ID NO: 28 - protein, 433 aa, Coprinopsis cinerea okayama7#130
QRPLYAQCGGTGWTGETTCVSGAVCEVINQWYHQCLPGSNQPQPPVTTQPPVVVPTTSQPPVVVPTNPPGGTPVPSTGNPFEGYDIYLSPYYAEEVEAAAAMIDDPVLKAKALKVKEIPTFIWFDVVRKTPDLGRYLADATAIQQRTGRKQLVQIVVYDLPDRDCAAAASNGEFSLADGGMEKYKDYVDRLASEIRKYPDVRIVAVIEPDSLANMVTNMNVAKCRGAEAAYKEGVIYALRQLSALGVYSYVDAGHAGWLGWNANLAPSARLFAQIYKDAGRSAFIRGLATNVSNYNALSATTRDPVTQGNDNYDELRFINALAPLLRNEGWDAKFIVDQGRSGVQNIRQEWGNWCNVYGAGFGMRPTLNTPSSAIDAIVWIKPGGEADGTSDTSAPRYDTHCGKSDSHKPAPEAGTWFQEYFVNLVKNANPPL

SEQ ID NO: 29 - protein, 361 aa, Artificial sequence
  Synthetic polypeptide of hypothetical protein MPER_09318 from Moniliophthora perniciosa FA553
IPGSDPGNPGPTSSSTLSSTAAPPTNTQSPVEDNPYTGYTIYLSPYYADEIDAAAAKITDPTLKVQALKVKEIPTFIWFDTTAKLSTLEPYLKDASAKGKAEGKKYLLQIVVYTLPERDCAALASNGELSIDNGGEVKSREYIDTMVATIKKYPDVRVVAVVEPDSLANLVTNLNVQKCSKAQTIYKTSTQYALKQLDTAGVYMYLDAGHAGWLGWPANLTPTAQLFQQVWQDAGSPKFVRGLATNVANFNALRAASPDPVTSQNPNYDEIHYIEGRAGQQNLRKEWGDWCNVKGAGFGTRPTTNTGSSLIDSIVWVKPGGESARFDAKCVSASSHVPAPEAGTWFQEYFEALVRNANPAL

SEQ ID NO: 30 - protein, 378 aa, Myceliophthora thermophila
APSRTTPQKPRQASAGCASAVTLDASTNVFQQYTLHPNNFYRAEVEAAAEAISDSALAEKARKVADVGTFLWLDTIENIGRLEPALEDVPCENIVGLVIYDLPGRDCAAKASNGELKVGELDRYKTEYIDKIAEILKAHSNTAFALVIEPDSLPNLVTNSDLQTCQQSASGYREGVAYALKQLNLPNVVMYIDAGHGGWLGWDANLKPGAQELASVYKSAGSPSQVRGISTNVAGWNAWDQEPGEFSDASDAQYNKCQNEKIYINTFGAELKSAGMPNHAIIDTGRNGVTGLRDEWGDWCNVNGAGFGVRPTANTGDELADAFVWVKPGGESDGTSDSSAARYDSFCGKPDAFKPSPEAGTWNQAYFEMLLKNANPSF

SEQ ID NO: 31 - protein, 366 aa, Artificial sequence
  Synthetic polypeptide of hypothetical protein SNOG_16444 from Phaeosphaeria nodorum SN15
APSPVENGPITARAVGAAAAACATPVTLSGNPFASRQIYANKFYSSEVSAAAAAMTDSALAASATKIDIVEDTIKDVPCDQIAALVIYDLPGRDCAAKASNGELPVGSLETYKTEYIDPIVAIFKKYPNIAIALVIEPDSLPNLVTNANLQTCKDSAEGYRKGVAYALKSLNLPNIAMYIDAGHGGWLGWNDNLKPGAKELATVYKDAGSPKQVRGVSTNVAGWNAYDLSPGEFSKATDAQYNKAQNEKLFVSMFSPELKSAGMPGQAIIDTARNGVTGLRKEWGDWCNVKGAGFGVRPTGNTGNTLVDAFVWVKPGGESDGTSDSSATRYDSFCGKDDAFKPSPEAGQWHQAYFEELVKNAKPAL

SEQ ID NO: 32 - protein, 436 aa, Trametes versicolor
MFKFAAAGQCGGVGWTGRTTCVSGSVCSKQNDYYSQCISGAGAPGTTVAPTTAPTAPATSAPGGSPTTVSAPSTPSSTPAAGNPFTGFQVYLSPYYSAEIASAAAAVTDSSLKAKAASVANIPTFTWLDSVAKVPDLGTYLADASSIQTKTGQKQLVPIVVYELPDRDCAAKASNGEFSIADAGAENYKDYIDQIVPQIKQFPDVRVVAVIEPDSLANLVTNLNVQKCANGGTYKASVTYALQQLSSVGVTMYMDAGHAGWLGWPANIQPGSEVFAEMFKSADFVAFVRAFATNVREYNALTAAFPRPITQGNPNYDEFPYIQRVRPMLKSPGFPAQFVVDQGRAGQQNFRQQWGDWCNIKGAGFGTRPTTSTGNPLIDAIIWVKPGGESDGTSNSSSPRYDSTLLSVRRDDPAPEAGTWFQAYFETLVSKPTRPL

SEQ ID NO: 33 - DNA, 2487 bp, Myceliophthora thermophila
atgaggacctcctctcgtttaatcggtgcccttgcggcggcactcttgccgtctgcccttgcgcagaacaacgcgccggtaaccttcaccgacccggactcgggcattaccttcaacacgtggggtctcgccgaggattctccccagactaagggcggtttcacttttggtgttgctctgccctctgatgccctcacgacagacgccaaggagttcatcggttacttgaaatgcgcgaggaacgatgagagcggttggtgcggtgtctccctgggcggccccatgaccaactcgctcctcatcgcggcctggccccacgaggacaccgtctacacctctctccgcttcgccaccggctatgccatgccggatgtctaccagggggacgccgagatcacccaggtctcctcctctgtcaactcgacgcacttcagcctcatcttcaggtgcgagaactgcctgcaatggagtcaaagcggcgccaccggcggtgcctccacctcgaacggcgtgttggtcctcggctgggtccaggcattcgccgaccccggcaacccgacctgccccgaccagatcaccctcgagcagcacgacaacggcatgggtatctggggtgcccagctcaactccgacgccgccagcccgtcctacaccgagtgggccgcccaggccaccaagaccgtcacgggtgactgcggcggtcccaccgagacctctgtcgtcggtgtccccgttccgacgggcgtctcgttcgattacatcgtcgtgggcggcggtgccggtggcatccccgccgccgacaagctcagcgaggccggcaagagtgtgctgctcatcgagaagggctttgcctcgaccgccaacaccggaggcactctcggccccgagtggctcgagggccacgaccttacccgctttgacgtgccgggtctgtgcaaccagatctgggttgactccaaggggatcgcttgcgaggataccgaccagatggctggctgtgtcctcggcggcggtaccgccgtgaatgccggcctgtggttcaagccctactcgctcgactgggactacctcttccctagtggttggaagtacaaagacgtccagccggccatcaaccgcgccctctcgcgcatcccgggcaccgatgctccctcgaccgacggcaagcgctactaccaacagggcttcgacgtcctctccaagggcctggccggcggcggctggacctcggtcacggccaataacgcgccagacaagaagaaccgcaccttctcccatgcccccttcatgttcgccggcggcgagcgcaacggcccgctgggcacctacttccagaccgccaagaagcgcagcaacttcaagctctggctcaacacgtcggtcaagcgcgtcatccgccagggcggccacatcaccggcgtcgaggtcgagccgttccgcgacggcggttaccaaggcatcgtccccgtcaccaaggttacgggccgcgtcatcctctctgccggtacctttggcagtgcaaagatcctgctgaggagcggtatcggtccgaacgatcagctgcaggttgtcgcggcctcggagaaggatggccctaccatgatcagcaactcgtcctggatcaacctgcctgtcggctacaacctggatgaccacctcaacaccgacactgtcatctcccaccccgacgtcgtgttctacgacttctacgaggcgtgggacaatcccatccagtctgacaaggacagctacctcaactcgcgcacgggcatcctcgcccaagccgctcccaacattgggcctatgttctgggaagagatcaagggtgcggacggcattgttcgccagctccagtggactgcccgtgtcgagggcagcctgggtgcccccaacggcaagaccatgaccatgtcgcagtacctcggtcgtggtgccacctcgcgcggccgcatgaccatcaccccgtccctgacaactgtcgtctcggacgtgccctacctcaaggaccccaacgacaaggaggccgtcatccagggcatcatcaacctgcagaacgccctcaagaacgtcgccaacctgacctggctcttccccaactcgaccatcacgccgcgccaatacgttgacagcatggtcgtctccccgagcaaccggcgctccaaccactggatgggcaccaacaagatcggcaccgacgacgggcgcaagggcggctccgccgtcgtcgacctcaacaccaaggtctacggcaccgacaacctcttcgtcatcgacgcctccatcttccccggcgtgcccaccaccaaccccacctcgtacatcgtgacggcgtcggagcacgcctcggcccgcatcctcgccctgcccgacctcacgcccgtccccaagtacgggcagtgcggcggccgcgaatggagcggcagcttcgtctgcgccgacggctccacgtgccagatgcagaacgagtggtactcgcagtgcttgtga

SEQ ID NO: 34 - protein, 828 aa, Myceliophthora thermophila
MRTSSRLIGALAAALLPSALAQNNAPVTFTDPDSGITFNTWGLAEDSPQTKGGFTFGVALPSDALTTDAKEFIGYLKCARNDESGWCGVSLGGPMTNSLLIAAWPHEDTVYTSLRFATGYAMPDVYQGDAEITQVSSSVNSTHFSLIFRCENCLQWSQSGATGGASTSNGVLVLGWVQAFADPGNPTCPDQITLEQHDNGMGIWGAQLNSDAASPSYTEWAAQATKTVTGDCGGPTETSVVGVPVPTGVSFDYIVVGGGAGGIPAADKLSEAGKSVLLIEKGFASTANTGGTLGPEWLEGHDLTRFDVPGLCNQIWVDSKGIACEDTDQMAGCVLGGGTAVNAGLWFKPYSLDWDYLFPSGWKYKDVQPAINRALSRIPGTDAPSTDGKRYYQQGFDVLSKGLAGGGWTSVTANNAPDKKNRTFSHAPFMFAGGERNGPLGTYFQTAKKRSNFKLWLNTSVKRVIRQGGHITGVEVEPFRDGGYQGIVPVTKVTGRVILSAGTFGSAKILLRSGIGPNDQLQVVAASEKDGPTMISNSSWINLPVGYNLDDHLNTDTVISHPDVVFYDFYEAWDNPIQSDKDSYLNSRTGILAQAAPNIGPMFWEEIKGADGIVRQLQWTARVEGSLGAPNGKTMTMSQYLGRGATSRGRMTITPSLTTVVSDVPYLKDPNDKEAVIQGIINLQNALKNVANLTWLFPNSTITPRQYVDSMVVSPSNRRSNHWMGTNKIGTDDGRKGGSAVVDLNTKVYGTDNLFVIDASIFPGVPTTNPTSYIVTASEHASARILALPDLTPVPKYGQCGGREWSGSFVCADGSTCQMQNEWYSQCL

SEQ ID NO: 35 - DNA, 2364 bp, Myceliophthora thermophila
atgaagctactcagccgcgttggggcgaccgccctagcggcgacgttgtcactgcagcaatgtgcagcccagatgaccgaggggacctacaccgatgaggctaccggtatccaattcaagacgtggaccgcctccgagggcgcccctttcacgtttggcttgaccctccccgcggacgcgctggaaaaggatgccaccgagtacattggtctcctgcgttgccaaatcaccgatcccgcctcgcccagctggtgcggtatctcccacggccagtccggccagatgacgcaggcgctgctgctggtcgcctgggccagcgaggacaccgtctacacgtcgttccgctacgccaccggctacacgctccccggcctctacacgggcgacgccaagctgacccagatctcctcctcggtcagcgaggacagcttcgaggtgctgttccgctgcgaaaactgcttctcctgggaccaggatggcaccaagggcaacgtctcgaccagcaacggcaacctggtcctcggccgcgccgccgcgaaggatggtgtgacgggccccacgtgcccggacacggccgagttcggtttccatgataacggtttcggacagtggggtgccgtgcttgagggtgctacttcggactcgtacgaggagtgggctaagctggccacgaccacgcccgagaccacctgcgatggcactggccccggcgacaaggagtgcgttccggctcccgaggacacgtatgattacatcgttgtcggtgccggcgccggtggtatcaccgtcgccgacaagctcagcgaggccggccacaaggtccttctcatcgagaagggacccccttcgaccggcctgtggaacgggaccatgaagcccgagtggctcgagagcaccgaccttacccgcttcgacgttcccggcctgtgcaaccagatctgggtcgactctgccggcatcgcctgcaccgataccgaccagatggcgggctgcgttctcggcggtggcaccgctgtcaacgctggtttgtggtggaagccccaccccgctgactgggatgagaacttccccgaagggtggaagtcgagcgatctcgcggatgcgaccgagcgtgtcttcaagcgcatccccggcacgtcgcacccgtcgcaggacggcaagttgtaccgccaggagggcttcgaggtcatcagcaagggcctggccaacgccggctggaaggaaatcagcgccaacgaggcgcccagcgagaagaaccacacctatgcacacaccgagttcatgttctcgggcggtgagcgtggcggccccctggcgacgtaccttgcctcggctgccgagcgcagcaacttcaacctgtggctcaacactgccgtccggagggccgtccgcagcggcagcaaggtcaccggcgtcgagctcgagtgcctcacggacggtggcttcagcgggaccgtcaacctgaatgagggcggtggtgtcatcttctcggccggcgctttcggctcggccaagctgctccttcgcagcggtatcggtcctgaggaccagctcgagattgtggcgagctccaaggacggcgagaccttcactcccaaggacgagtggatcaacctccccgtcggccacaacctgatcgaccatctcaacactgacctcattatcacgcacccggatgtcgttttctatgacttctatgcggcctgggacgagcccatcacggaggataaggaggcctacctgaactcgcggtccggcattctcgcccaggcggcgcccaatatcggccctatgatgtgggatcaagtcacgccgtccgacggcatcacccgccagttccagtggacatgccgtgttgagggcgacagctccaagaccaactcgacccacgccatgaccctcagccagtacctcggccgtggcgtcgtctcgcgcggccggatgggcatcacctccgggctgagcacgacggtggccgagcacccgtacctgcacaacaacggcgacctggaggcggtcatccaggggatccagaacgtggtggacgcgctcagccaggtggccgacctcgagtgggtgctcccgccgcccgacgggacggtggccgactacgtcaacagcctgatcgtctcgccggccaaccgccgggccaaccactggatgggcacggccaagctgggcaccgacgacggccgctcgggcggcacctcggtcgtcgacctcgacaccaaggtgtacggcaccgacaacctgttcgtcgtcgacgcgtccgtcttccccggcatgtcgacgggcaacccgtcggccatgatcgtcatcgtggccgagcaggcggcgcagcgcatcctggccctgcggtcttaa

SEQ ID NO: 36 - protein, 787 aa, Myceliophthora thermophila
MKLLSRVGATALAATLSLQQCAAQMTEGTYTDEATGIQFKTWTASEGAPFTFGLTLPADALEKDATEYIGLLRCQITDPASPSWCGISHGQSGQMTQALLLVAWASEDTVYTSFRYATGYTLPGLYTGDAKLTQISSSVSEDSFEVLFRCENCFSWDQDGTKGNVSTSNGNLVLGRAAAKDGVTGPTCPDTAEFGFHDNGFGQWGAVLEGATSDSYEEWAKLATTTPETTCDGTGPGDKECVPAPEDTYDYIVVGAGAGGITVADKLSEAGHKVLLIEKGPPSTGLWNGTMKPEWLESTDLTRFDVPGLCNQIWVDSAGIACTDTDQMAGCVLGGGTAVNAGLWWKPHPADWDENFPEGWKSSDLADATERVFKRIPGTSHPSQDGKLYRQEGFEVISKGLANAGWKEISANEAPSEKNHTYAHTEFMFSGGERGGPLATYLASAAERSNFNLWLNTAVRRAVRSGSKVTGVELECLTDGGFSGTVNLNEGGGVIFSAGAFGSAKLLLRSGIGPEDQLEIVASSKDGETFTPKDEWINLPVGHNLIDHLNTDLIITHPDVVFYDFYAAWDEPITEDKEAYLNSRSGILAQAAPNIGPMMWDQVTPSDGITRQFQWTCRVEGDSSKTNSTHAMTLSQYLGRGVVSRGRMGITSGLSTTVAEHPYLHNNGDLEAVIQGIQNVVDALSQVADLEWVLPPPDGTVADYVNSLIVSPANRRANHWMGTAKLGTDDGRSGGTSVVDLDTKVYGTDNLFVVDASVFPGMSTGNPSAMIVIVAEQAAQRILALRS

SEQ ID NO: 37 - DNA, 1395 bp, Artificial sequence
  Synthetic DNA which is cDNA sequence encoding Myceliophthora thermophila wild-type cellobiohydrolase type 2b without signal peptide
gcccccgtcattgaggagcgccagaactgcggcgctgtgtggactcaatgcggcggtaacgggtggcaaggtcccacatgctgcgcctcgggctcgacctgcgttgcgcagaacgagtggtactctcagtgcctgcccaacagccaggtgacgagttccaccactccgtcgtcgacttccacctcgcagcgcagcaccagcacctccagcagcaccaccaggagcggcagctcctcctcctcctccaccacgcccccgcccgtctccagccccgtgaccagcattcccggcggtgcgacctccacggcgagctactctggcaaccccttctcgggcgtccggctcttcgccaacgactactacaggtccgaggtccacaatctcgccattcctagcatgactggtactctggcggccaaggcttccgccgtcgccgaagtccctagcttccagtggctcgaccggaacgtcaccatcgacaccctgatggtccagactctgtcccaggtccgggctctcaataaggccggtgccaatcctccctatgctgcccaactcgtcgtctacgacctccccgaccgtgactgtgccgccgctgcgtccaacggcgagttttcgattgcaaacggcggcgccgccaactacaggagctacatcgacgctatccgcaagcacatcattgagtactcggacatccggatcatcctggttatcgagcccgactcgatggccaacatggtgaccaacatgaacgtggccaagtgcagcaacgccgcgtcgacgtaccacgagttgaccgtgtacgcgctcaagcagctgaacctgcccaacgtcgccatgtatctcgacgccggccacgccggctggctcggctggcccgccaacatccagcccgccgccgagctgtttgccggcatctacaatgatgccggcaagccggctgccgtccgcggcctggccactaacgtcgccaactacaacgcctggagcatcgcttcggccccgtcgtacacgtcgcctaaccctaactacgacgagaagcactacatcgaggccttcagcccgctcttgaactcggccggcttccccgcacgcttcattgtcgacactggccgcaacggcaaacaacctaccggccaacaacagtggggtgactggtgcaatgtcaagggcaccggctttggcgtgcgcccgacggccaacacgggccacgagctggtcgatgcctttgtctgggtcaagcccggcggcgagtccgacggcacaagcgacaccagcgccgcccgctacgactaccactgcggcctgtccgatgccctgcagcctgcccccgaggctggacagtggttccaggcctacttcgagcagctgctcaccaacgccaacccgcccttc

SEQ ID NO: 38 - protein, 433 aa, Artificial sequence
  Synthetic polypeptide consensus sequence
APVIEERQCASVWGQCGGGWNGPTCCSGSTCVQNDWYSQCLPGVTTSSTSTSSSSSSSSTSSTTSTSSTTPTSIPGGASSTASYSGNPFGVQLWANYYRSEVHTLAIPSITDPALAAKAAAVAEVPSFQWLDRNVTVDTLLTLSEIRAANQAGANPPYAAQIVVYDLPDRDCAAAASNGEWSIANNGAANYKAYIDRIREILIYSDIRTILVIEPDSLANMVTNMNVKCSGAASTYRELTIYALKQLNLPNVAMYMDAGHAGWLGWPANIQPAAELFAIYKDAGKPAVRGLATNVANYNAWSISSPPSYTSPNPNYDEKHYIEAFSPLLAAGFPAFIVDTGRSGKQPTGQEWGHWCNAIGTGFGMRPTANTGHELVDAFVWVKPGGECDGTSDTSAARYDYHCGLDALKPAPEAGQWFQAYFEQLLTNANPPF

SEQ ID NO: 39 - protein, 319 aa, Artificial sequence
  Synthetic polypeptide consensus sequence
WGQCGGWTGTCSGCNYYQCLPGSSSTTSTTTSGNPFGQLYNPYYASEVAAIPITALAAKAAAVAVPTFWLDAKVPLYLADIANAGGNLGQIVVYDLPDRDCAAASNGEFSIAGLKYKYIDIAIYDVRVVLVIEPDSLANLVTNLNVKCANASAYKEYALQLNLVMYLDAGHAGWLGWPANLPAALFAVYKAGPVRGLATNVANYNAWSSPPTGNNYDEYIALAPLLGFPAFIVDQGRSGVQPQWGDWCNVGAGFGVRPTTNTGSLIDAFVWVKPGGESDGTSDTSARYDSHCGLSDALPAPEAGTWFQAYFELLNANPA

SEQ ID NO: 40 - protein, 17 aa, Myceliophthora thermophila
MAKKLFITAALAAAVLA

(19) United States
(12) Patent Application Publication
(10) Pub. No.: US 2012/0276594 A1
(43) Pub. Date: Nov. 1, 2012

(54) CELLOBIOHYDROLASE VARIANTS

(75) Inventors: Rama Voladri, Redwood City, CA (US); Xiyun Zhang, Redwood City, CA (US); Sachin Patil, Redwood City, CA (US); David Elgart, Redwood City, CA (US); Gregory Miller, Redwood City, CA (US); Louis Clark, Redwood City, CA (US); Kui Chan, Redwood City, CA (US)

(73) Assignee: Codexis, Inc., Redwood City, CA (US)

(21) Appl. No.: 13/459,038

(22) Filed: Apr. 27, 2012

Related U.S. Application Data
(60) Provisional application No. 61/613,827, filed on Mar. 21, 2012.
(60) Provisional application No. 61/479,800, filed on Apr. 27, 2011.

(51) Int. Cl. C12N 9/42; C12N 1/19; C12N 1/15; C12P 19/14; C12P 7/14; C12P 7/16; C12P 7/48; C12P 7/54; C12P 7/56; C12P 7/58; C12P 7/46; C12P 7/20; C12P 7/18; C12P 13/14; C12N 15/56; C12N 15/80

(52) U.S. Cl. 435/99; 435/209; 435/320.1; 435/254.2; 435/254.11; 435/162; 435/160; 435/144; 435/140; 435/139; 435/137; 435/145; 435/159; 435/158; 435/110; 536/23.2

(57) ABSTRACT

The present invention relates to cellobiohydrolase variants having improved thermostability and/or thermoactivity in comparison to wild-type Myceliophthora thermophila CBH2b.

### CROSS-REFERENCES TO RELATED APPLICATIONS

This application claims benefit of priority of U.S. Provisional Application No. 61/479,800, filed Apr. 27, 2011, and of U.S. Provisional Application No. 61/613,827, filed Mar. 21, 2012, the entire content of each of which is incorporated herein by reference.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 90834-836557_ST25.TXT, created on Apr. 27, 2012, 151,371 bytes, machine format IBM-PC, MS-Windows operating system, is hereby incorporated by reference in its entirety for all purposes.

### FIELD OF THE INVENTION

This invention relates to cellobiohydrolase variants and their use in the production of fermentable sugars from cellulosic biomass.

### BACKGROUND OF THE INVENTION

Cellulosic biomass is a significant renewable resource for the generation of fermentable sugars. These sugars can be used as reactants in various metabolic processes, including fermentation, to produce biofuels, chemical compounds, and other commercially valuable products. While the fermentation of simple sugars such as glucose to ethanol is relatively straightforward, the efficient conversion of cellulosic biomass to fermentable sugars is challenging (see, e.g., Ladisch et al., 1983, Enzyme Microb. Technol. 5:82). Cellulose may be pretreated chemically, mechanically, enzymatically or in other ways to increase the susceptibility of cellulose to hydrolysis. Such pretreatment may be followed by the enzymatic conversion of cellulose to cellobiose, cello-oligosaccharides, glucose, and other sugars and sugar polymers, using enzymes that break down the β-1-4 glycosidic bonds of cellulose. These enzymes are collectively referred to as “cellulases.”

Cellulases are divided into three sub-categories of enzymes: 1,4-β-D-glucan glucanohydrolase (“endoglucanase” or “EG”); 1,4-β-D-glucan cellobiohydrolase (“exoglucanase,” “cellobiohydrolase,” or “CBH”); and β-D-glucoside-glucohydrolase (“β-glucosidase,” “cellobiase,” or “BGL”). See Methods in Enzymology, 1988, Vol. 160, p. 200-391 (Eds. Wood, W. A. and Kellogg, S.T.). These enzymes act in concert to catalyze the hydrolysis of cellulose-containing substrates. Endoglucanases break internal bonds and disrupt the crystalline structure of cellulose, exposing individual cellulose polysaccharide chains (“glucans”). Cellobiohydrolases incrementally shorten the glucan molecules, releasing mainly cellobiose units (a water-soluble β-1,4-linked dimer of glucose) as well as glucose, cellotriose, and cellotetrose. β-glucosidases split the cellobiose into glucose monomers.

Cellulases with improved properties for use in processing cellulosic biomass would reduce costs and increase the efficiency of production of biofuels and other commercially valuable compounds.

### BRIEF SUMMARY OF THE INVENTION

In one aspect, the present invention provides recombinant cellobiohydrolase variants that exhibit improved properties. In some embodiments, the cellobiohydrolase variants are superior to naturally occurring cellobiohydrolases under conditions required for saccharification of cellulosic biomass.

In some embodiments, a recombinant cellobiohydrolase variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:1 and comprises an amino acid substitution, relative to SEQ ID NO:1, at one or more positions selected from 1, 7, 27, 73, 99, 100, 111, 119, 120, 121, 126, 128, 151, 165, 168, 169, 227, 230, 245, 250, 251, 253, 260, 267, 272, 276, 286, 289, 292, 294, 295, 297, 301, 311, 325, 327, 333, 334, 336, 339, 341, 353, 359, 360, 363, 381, 382, 384, 397, 403, 405, 424, 425, 426, 429, 432, 436, 437, 441, 448, 459, and 464, wherein the position is numbered with reference to SEQ ID NO:1. In some embodiments, the variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:1 and comprises an amino acid substitution, relative to SEQ ID NO:1, at one or more positions selected from A1, R7, C27, T73, A99, T100, S111, D119, Y120, Y121, H126, L128, Q151, Q165, S168, Q169, I227, S230, N245, M250, N251, A253, S260, V267, Q272, P276, H286, W289, W292, A294, N295, Q297, E301, G311, N325, N327, S333, A334, S336, S339, N341, F353, S359, A360, P363, Q381, Q382, G384, R397, G403, E405, D424, T425, S426, R429, Y432, L436, S437, Q441, Q448, T459, and P464, wherein the position is numbered with reference to SEQ ID NO:1. In some embodiments, the variant comprises one or more amino acid substitutions selected from A1V, R7S, C27Y, T73A, A99P, T100G/N, S111N, D119P/R, Y120H, Y121R, H126E, L128H, Q151L, Q165P/R, S168T, Q169K/L/R, I227A/G/H/K/M/Q, S230P, N245T, M250G, N251D/T, A253P/T, S260K, V267E/K/L, Q272R, P276T, H286Q/S, W289C/M/S, W292A/H/P/R, A294R, N295R, Q297K/P/R/Y, E301K, G311Q, N325H, N327L, S333F, A334P, S336H/K/N/P/T, S339R/Q/W, N341V, F353I, S359D/K, A360C/K/T, P363D/H/V, Q381L, Q382R, G384T, R397H, G403T, E405G/P, D424N/Q, T425K/P/R, S426K, R429D/H/N, Y432W, L436K, S437G/P, Q441K, Q448K, T459G/K/N/R, and P464R. In some embodiments, a recombinant cellobiohydrolase variant is encoded by a polynucleotide that hybridizes at high stringency to the complement of SEQ ID NO:37 and comprises one or more amino acid substitutions as described herein. In some embodiments, the variant has an improved property relative to wild-type M. thermophila CBH2b (SEQ ID NO:1). In some embodiments, the variant has increased thermostability in comparison to wild-type M. thermophila CBH2b (SEQ ID NO:1).

In some embodiments, the variant comprises an amino acid substitution at one or more positions selected from A99, S230, A253, A334, E405, and S437. In some embodiments, the variant comprises one or more amino acid substitutions selected from A99P, S230P, A253P/T, A334P, E405P, and S437P.

In some embodiments, the variant comprises an amino acid substitution at one or more positions selected from R7, T100, Y120, Q169, I227, A253, Q297, E301, S336, S339, A360, and T459. In some embodiments, the variant comprises one or more amino acid substitutions selected from R7S, T100G, Y120H, Q169R, I227M, A253T, Q297K, E301K, S336K/N/T, S339W, A360T, and T459N/R/G.

In some embodiments, the variant comprises an amino acid substitution at one or more positions selected from Y120, I227, E301, and T459. In some embodiments, the variant comprises one or more amino acid substitutions selected from Y120H, I227M, E301K, and T459N/R.

In some embodiments, the variant comprises the amino acid substitutions S230P, A253P, E405P, and S437P. In some embodiments, the variant has the amino acid sequence of SEQ ID NO:2. In some embodiments, the variant comprises the amino acid substitutions R7S, T100G, Y120H, Q165R, S230P, A253P, S339Q, E405P, S437P, and T459N. In some embodiments, the variant has the amino acid sequence of SEQ ID NO:3. In some embodiments, the variant comprises the amino acid substitutions R7S, T100G, Y120H, Q165R, I227M, S230P, A253P, S339Q, E405P, S437P, and T459N. In some embodiments, the variant has the amino acid sequence of SEQ ID NO:4.

In some embodiments, a recombinant cellobiohydrolase variant comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises one or more pairs of amino acid substitutions, relative to SEQ ID NO:1, selected from P109C and A279C, A129C and Q451C, I159C and A221C, V247C and A299C, A304C and A360C, L128C and W449C, A284C and L319C, I219C and A269C, I207C and T261C, A300C and L356C, and V267C and D309C, wherein the position is numbered with reference to SEQ ID NO:1. In some embodiments, a recombinant cellobiohydrolase variant is encoded by a polynucleotide that hybridizes at high stringency to the complement of SEQ ID NO:37 and comprises one or more pairs of amino acid substitutions as described herein.

In some embodiments, the variant has increased thermostability and/or thermoactivity in comparison to wild-type M. thermophila CBH2b (SEQ ID NO:1). In some embodiments, the variant exhibits at least a 1.1-fold increase in thermostability relative to wild-type M. thermophila CBH2b (SEQ ID NO:1). In some embodiments, the variant exhibits at least a 3.0-fold increase in thermostability relative to wild-type M. thermophila CBH2b (SEQ ID NO:1). In some embodiments, the variant has increased thermostability after incubation at pH 4.5 and 67° C. for 1 hour in comparison to wild-type M. thermophila CBH2b (SEQ ID NO:1).

In some embodiments, a recombinant cellobiohydrolase variant comprises at least about 50% (or at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%) sequence identity to SEQ ID NO:1 and comprises one or more amino acid substitutions, relative to SEQ ID NO:1, selected from:

an aspartic acid, isoleucine, lysine, asparagine, arginine, serine, or threonine residue at position 92 (X92D/I/K/N/R/S/M;

an asparagine or proline residue at position 94 (X94N/P);

a histidine, leucine, or asparagine residue at position 95 (X95H/L/N);

a glutamic acid, phenylalanine, isoleucine, or serine residue at position 96 (X96E/F/I/S);

a cysteine or asparagine residue at position 111 (X111C/N);

an alanine, cysteine, lysine, proline, arginine, or valine residue at position 119 (X119A/C/K/P/R/V);

a lysine, asparagine, or serine residue at position 161 (X161K/N/S);

an alanine, leucine, or arginine residue at position 176 (X176G/L/R);

a glycine, histidine, glutamine, or serine residue at position 213 (X213G/H/Q/S);

an aspartic acid, histidine, or serine residue at position 249 (X249D/H/S);

a cysteine, glycine, leucine, or methionine residue at position 250 (X250C/G/L/M);

a cysteine, methionine, serine, or threonine residue at position 289 (X289C/M/S/T);

a glutamine, arginine, or tryptophan residue at position 294 (X294Q/R/W);

an alanine, cysteine, glutamic acid, histidine, lysine, leucine, asparagine, proline, threonine, or valine residue at position 336 (X336A/C/E/H/K/L/N/P/T/V);

an alanine or glutamic acid residue at position 358 (X358A/E);

an alanine, aspartic acid, lysine, or tyrosine residue at position 359 (X359A/D/K/Y);

a methionine, serine, or threonine residue at position 384 (X384M/S/T);

a serine or threonine residue at position 427 (X427S/T);

a glutamic acid, proline, or tryptophan residue at position 432 (X432E/P/W); and

a glutamic acid, lysine, glutamine, or threonine residue at position 448 (X448E/K/Q/T),

wherein the position is numbered with reference to the amino acid sequence of SEQ ID NO:1, and wherein the variant has increased thermostability and/or thermoactivity in comparison to wild-type M. thermophila CBH2b (SEQ ID NO:1). In some embodiments, the variant comprises an alanine, cysteine, glutamic acid, histidine, lysine, leucine, asparagine, proline, threonine, or valine residue at position 336 (X336A/C/E/H/K/L/N/P/T/V). In some embodiments, a recombinant cellobiohydrolase variant is encoded by a polynucleotide that hybridizes at high stringency to the complement of SEQ ID NO:37 and comprises one or more amino acid substitutions as described herein.

In some embodiments, a recombinant cellobiohydrolase variant comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises an amino acid substitution, relative to SEQ ID NO:1, at one or more positions selected from 2, 6, 7, 8, 12, 14, 18, 20, 21, 29, 33, 36, 37, 40, 47, 49, 50, 56, 61, 64, 67, 74, 76, 81, 83, 86, 87, 92, 94, 95, 96, 99, 100, 101, 102, 106, 107, 112, 113, 117, 118, 120, 123, 126, 128, 130, 132, 133, 139, 142, 143, 146, 151, 157, 159, 160, 161, 162, 163, 164, 165, 166, 168, 169, 176, 178, 179, 181, 206, 209, 210, 212, 213, 224, 227, 228, 230, 243, 247, 248, 249, 252, 253, 256, 259, 260, 267, 271, 272, 297, 308, 311, 312, 332, 336, 339, 340, 341, 353, 354, 356, 358, 359, 360, 363, 364, 365, 382, 384, 396, 400, 401, 404, 405, 427, 428, 436, 437, 445, 448, and 459, wherein the position is numbered with reference to SEQ ID NO:1. In some embodiments, the variant comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises an amino acid substitution, relative to SEQ ID NO:1, at one or more positions selected from P2, E6, R7, Q8, A12, W14, G18, N20, G21, A29, T33, A36, Q37, W40, N47, Q49, V50, P56, T61, R64, S67, R74, G76, S81, T83, P86, P87, V92, S94, I95, P96, A99, T100, S101, T102, S106, G107, G112, V113, A117, N118, Y120, S123, H126, L128, I130, S132, M133, A139, S142, A143, E146, Q151, V157, I159, D160, T161, L162, M163, V164, Q165, T166, S168, Q169, A176, A178, N179, P181, S206, N209, G210, A212, A213, K224, I227, E228, S230, M243, V247, T248, N249, V252, A253, S256, A259, S260, V267, K271, Q272, Q297, N308, G311, K312, A332, S336, S339, P340, N341, F353, S354, L356, N358, S359, A360, P363, A364, R365, Q382, G384, V396, A400, N401, H404, E405, A427, A428, L436, S437, E445, Q448, and T459, wherein the position is numbered with reference to SEQ ID NO:1. In some embodiments, the variant comprises one or more amino acid substitutions selected from P2H/S, E6N, R7H/S, Q8L/P, A12I, W14L, G18D, N20L/S, G21D/K, A29R/T, T33H, A36E, Q37F/H/L, W40L, N47K, Q49K, V50D/E/H/K/R, P56T, T61A, R64C, S67G, R74S, G76D, S81P, T83D, P86T, P87T, V92D/K/R/S, S94N, I95H/N, P96E/S, A99V, T100V, S101G, T102C/W, S106W/Y, G107D, G112E, V113I, A117T, N118D, Y120E/N/R, S123R/Y, H126E/L/M, L128E/H, I130V, S1321, M133F/V, A139H/T, S142E, A143M, E146L, Q1511/L, V157D/H/S, I159S, D160H, T161N/S, L162I, M163A/L, V164E/R, Q165P/T, T166R, S168G/Q/R, Q169D/R, A176G/R, A178N, N179D, P181A, S206H/K, N209S, G210A, A212C/L/N/P/R/S, A213G/H/Q, K224A/E/W, I227A/H/K/M/T, E228G, S230P, M243I, V247A, T248S, N249D/S, V252N, A253N/P/T, S256R, A259E, S260D/K, V267L, K271A, Q272H, Q297R, N308E, G311D, K312A, A332S, S336A/E/L/N/T, S339E/L/Q/V, P340N, N341D, F353L, S354G, L356E/G/H, N358E, S359D/Y, A360D/E/Q/R/S/T/V, P363D, A364T, R365G/L, Q382A/D/H/R, G384S, V396E/R, A400V, N401D, H404N, E405P/Q, A427T, A428N/S, L436D/N, S437P, E445D, Q448T, and T459R. In some embodiments, a recombinant cellobiohydrolase variant is encoded by a polynucleotide that hybridizes at high stringency to the complement of SEQ ID NO:37 and encodes a protein that comprises one or more amino acid substitutions as described herein. In some embodiments, the variant has an improved property relative to wild-type M. thermophila CBH2b (SEQ ID NO:1). In some embodiments, the variant has increased activity in generating glucose in comparison to wild-type M. thermophila CBH2b (SEQ ID NO:1) in a thermoactivity assay.

In some embodiments, the variant comprises an amino acid substitution at one or more positions selected from P86, H126, L128, Q165, Q169, A212, I227, S339, S359, and Q382. In some embodiments, the variant comprises one or more amino acid substitutions selected from P86T, H126M, L128H, Q165P/T, Q169R, A212S, I227H/K, S339Q, S359D, and Q382D.

In some embodiments, the variant comprises an amino acid substitution at one or more positions selected from P86, H126, Q165, Q169, A212, I227, S339, and S359. In some embodiments, the variant comprises one or more amino acid substitutions selected from P86T, H126M, Q165T, Q169R, A212S, I227H/K, S339Q, and S359D.

In some embodiments, the variant comprises an amino acid substitution at one or more positions selected from E6, Q8, P86, H126, L162, Q165, Q169, A212, I227, N249, A253, K271, S339, P340, S359, A360, N365, and Q382. In some embodiments, the variant comprises one or more amino acid substitutions selected from E6N, Q8P, P86T, H126M, L162I, Q165P, Q169R, A212S, I227K, N249S, A253N, K271A, S339Q, P340N, S359D, A360D, R365G, and Q382D.

In some embodiments, the variant comprises the amino acid substitutions Q165P/T and Q169D/R. In some embodiments, the variant comprises the amino acid substitutions H126M, Q165T, Q169R, A212S, I227H, and S339Q. In some embodiments, the variant comprises the amino acid substitutions P86T, Q165P, and Q169R. In some embodiments, the variant comprises the amino acid substitutions Q165P, Q169R, I227K, and S359D.

In some embodiments, a recombinant cellobiohydrolase variant comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises an amino acid substitution, relative to SEQ ID NO:1, at one or more positions selected from 165, 169, and 359, wherein the position is numbered with reference to SEQ ID NO:1. In some embodiments, the starting amino acid residue at position 165 is glutamine (Q165), the starting amino acid residue at position 169 is glutamine (Q169), and/or the starting amino acid residue at position 359 is serine (S359). In some embodiments, the amino acid residue at Q165 is replaced with proline (Q165P), the amino acid residue at Q169 is replaced with arginine (Q169R), and/or the amino acid residue at position S359 is replaced with aspartic acid (S359D). In some embodiments, the substituted amino acid residue at position 165 is proline, arginine, or threonine (X165P/R/T); the substituted amino acid residue at position 169 is aspartic acid, lysine, leucine, or arginine (X169D/K/L/R); and/or the substituted amino acid residue at position 359 is aspartic acid, lysine, or tyrosine (X359D/K/Y). In some embodiments, the substituted amino acid residue at position 165 is proline (X165P), the substituted amino acid residue at position 169 is arginine (X169R), and/or the substituted amino acid residue at position 359 is aspartic acid (X359D).

In some embodiments, the variant further comprises an amino acid substitution, relative to SEQ ID NO:1, at one or more positions selected from 126, 128, 227, 339, and 360. In some embodiments, the starting amino acid residue at position 126 is histidine (H126), the starting amino acid residue at position 128 is leucine (L128), the starting amino acid residue at position 227 is isoleucine (1227), the starting amino acid residue at position 339 is serine (S339), and/or the starting amino acid residue at position 360 is alanine (A360). In some embodiments, the amino acid residue at H126 is replaced with methionine (H126M), the amino acid residue at L128 is replaced with glutamic acid or histidine (L128E/H), the amino acid residue at 1227 is replaced with lysine (I227K), the amino acid residue at S339 is replaced with glutamic acid or glutamine (S339E/Q), and/or the amino acid residue at position A360 is replaced with aspartic acid (A360D). In some embodiments, the substituted amino acid residue at position 126 is glutamic acid, leucine, or methionine (X126E/L/M), the substituted amino acid residue at position 128 is glutamic acid or histidine (X128E/H), the substituted amino acid residue at position 227 is alanine, glycine, histidine, lysine, methionine, glutamine, or threonine (X227A/G/H/K/M/Q/T), the substituted amino acid residue at position 339 is glutamic acid, leucine, glutamine, arginine, valine, or tryptophan (X339E/L/Q/R/V/W), and/or the substituted amino acid residue at position 360 is cysteine, aspartic acid, glutamic acid, lysine, glutamine, arginine, serine, threonine, or valine (X360C/D/E/K/Q/R/S/T/V). In some embodiments, the substituted amino acid residue at position 126 is methionine (X126M), the substituted amino acid residue at position 128 is glutamic acid or histidine (X128E/H), the substituted amino acid residue at position 227 is lysine (X227K), the substituted amino acid residue at position 339 is glutamic acid or glutamine (X339E/Q), and/or the substituted amino acid residue at position A360 is aspartic acid (X360D).

In some embodiments, the variant further comprises an amino acid substitution, relative to SEQ ID NO:1, at one or more positions selected from 64, 86, 87, 102, 206, 212, 230, 253, 267, 271, 311, 332, 336, 340, 382, and 429. In some embodiments, the starting amino acid residue at position 64 is arginine (R64), the starting amino acid residue at position 86 is proline (P86), the starting amino acid residue at position 87 is proline (P87), the starting amino acid residue at position 102 is threonine (T102), the starting amino acid residue at position 206 is serine (S206), the starting amino acid residue at position 212 is alanine (A212), the starting amino acid residue at position 230 is serine (S230), the starting amino acid residue at position 253 is alanine (A253), the starting amino acid residue at position 267 is valine (V267), the starting amino acid residue at position 271 is lysine (K271), the starting amino acid residue at position 311 is glycine (G311), the starting amino acid residue at position 332 is alanine (A332), the starting amino acid residue at position 336 is serine (S336), the starting amino acid residue at position 340 is proline (P340), the starting amino acid residue at position 382 is glutamine (Q382), and/or the starting amino acid residue at position 429 is arginine (R429). In some embodiments, the amino acid residue at R64 is replaced with cysteine (R64C), the amino acid residue at P86 is replaced with threonine (P86T), the amino acid residue at P87 is replaced with threonine (P87T), the amino acid residue at T102 is replaced with cysteine (T102C), the amino acid residue at S206 is replaced with histidine or lysine (S206H/K), the amino acid residue at A212 is replaced with cysteine, leucine, asparagine, proline, arginine, or serine (A212C/L/N/P/R/S), the amino acid residue at S230 is replaced with proline (S230P), the amino acid residue at A253 is replaced with threonine (A253T), the amino acid residue at V267 is replaced with leucine (V267L), the amino acid residue at K271 is replaced with alanine (K271A), the amino acid residue at G311 is replaced with glutamine (G311Q), the amino acid residue at A332 is replaced with serine (A332S), the amino acid residue at S336 is replaced with asparagine (S336N), the amino acid residue at P340 is replaced with asparagine (P340N), the amino acid residue at Q382 is replaced with aspartic acid (Q382D), and/or the amino acid residue at R429 is replaced with asparagine (R429N). In some embodiments, the substituted amino acid residue at position 64 is cysteine (X64C), the substituted amino acid residue at position 86 is threonine (X86T), the substituted amino acid residue at position 87 is threonine (X87T), the substituted amino acid residue at position 102 is cysteine or tryptophan (X102C/W), the substituted amino acid residue at position 206 is histidine or lysine (X206H/K), the substituted amino acid residue at position 212 is cysteine, leucine, asparagine, proline, arginine, or serine (X212C/L/N/P/R/S), the substituted amino acid residue at position 230 is proline (X230P), the substituted amino acid residue at position 253 is asparagine, proline, or threonine (X253N/P/T), the substituted amino acid residue at position 267 is glutamic acid, lysine, or leucine (X267E/K/L), the substituted amino acid residue at position 271 is alanine (X271A), the substituted amino acid residue at position 311 is aspartic acid or glutamine (X311D/Q), the substituted amino acid residue at position 332 is serine (X332S), the substituted amino acid residue at position 336 is alanine, glutamic acid, histidine, lysine, leucine, asparagine, proline, or threonine (X336A/E/H/K/L/N/P/T), the substituted amino acid residue at position 340 is asparagine (X340N), the substituted amino acid residue at position 382 is alanine, aspartic acid, histidine, or arginine (X382A/D/H/R), and/or the substituted amino acid residue at position 429 is aspartic acid, histidine, or asparagine (X429D/H/N). In some embodiments, the substituted amino acid residue at position 64 is cysteine (X64C), the substituted amino acid residue at position 86 is threonine (X86T), the substituted amino acid residue at position 87 is threonine (X87T), the substituted amino acid residue at position 102 is cysteine (X102C), the substituted amino acid residue at position 206 is histidine or lysine (X206H/K), the substituted amino acid residue at position 212 is cysteine, leucine, asparagine, proline, arginine, or serine (X212C/L/N/P/R/S), the substituted amino acid residue at position 230 is proline (X230P), the substituted amino acid residue at position 253 is threonine (X253T), the substituted amino acid residue at position 267 is leucine (X267L), the substituted amino acid residue at position 271 is alanine (X271A), the substituted amino acid residue at position 311 is glutamine (X311Q), the substituted amino acid residue at position 332 is serine (X332S), the substituted amino acid residue at position 336 is asparagine (X336N), the substituted amino acid residue at position 340 is asparagine (X340N), the substituted amino acid residue at position 382 is aspartic acid (X382D), and/or the substituted amino acid residue at position 429 is asparagine (X429N).

In some embodiments, the variant has increased activity in generating glucose in comparison to wild-type M. thermophila CBH2b (SEQ ID NO:1) in a thermoactivity assay using a biomass substrate, such as an acid pre-treated wheat straw substrate. In some embodiments, the variant exhibits at least a 5% improvement in glucose production compared to wild-type M. thermophila CBH2b after incubation with a biomass substrate at 55° C. for 72 hours.

In some embodiments, the variant comprises at least about 50% (or at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%) sequence identity to a cellobiohydrolase type 2 from M. thermophila (SEQ ID NOs:1 or 30), Humicola insolens (SEQ ID NOs:5, 7, or 9), Chaetomium thermophilum (SEQ ID NO:6), Chaetomium globosum (SEQ ID NO:8), Podospora anserina (SEQ ID NO:10), Sordaria macrospora (SEQ ID NO:11), Botryotinia fuckeliana (SEQ ID NO:12), Nectria haematococca (SEQ ID NO:13), Aspergillus fumigatus (SEQ ID NO:14), Trichoderma reesei (SEQ ID NO:15), Gibberella zeae (SEQ ID NO:16), Magnaporthe oryzae (SEQ ID NO:17), Pyrenophora tritici-repentis (SEQ ID NO:18), Verticillium albo-atrum (SEQ ID NOs:19 or 27), Phaetosphaeria nodorum (SEQ ID NOs:20 or 31), Agaricus bisporus (SEQ ID NO:21), Volvariella volvacea (SEQ ID NO:22), Coniophora puteana (SEQ ID NOs:23 or 26), Phaenerochaete chrysosporium (SEQ ID NO:24), Lentinus sajor-caju (SEQ ID NO:25), Coprinopsis cinerea (SEQ ID NO:28), Moniliophthora perniciosa (SEQ ID NO:29), or Trametes versicolor (SEQ ID NO:32).

In some embodiments, the variant is a Myceliophthora thermophila cellobiohydrolase. In some embodiments, the variant is derived from a Myceliophthora thermophila type 2 cellobiohydrolase (e.g., a M. thermophila CBH2b of SEQ ID NO:1 or a M. thermophila CBH2a of SEQ ID NO: 30).

In another aspect, the present invention provides polynucleotides encoding cellobiohydrolase variants that exhibit improved properties. In some embodiments, the polynucleotide encodes an amino acid sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises an amino acid substitution, relative to SEQ ID NO:1, at one or more positions selected from 1, 7, 27, 73, 99, 100, 111, 119, 120, 121, 126, 128, 151, 165, 168, 169, 227, 230, 245, 250, 251, 253, 260, 267, 272, 276, 286, 289, 292, 294, 295, 297, 301, 311, 325, 327, 333, 334, 336, 339, 341, 353, 359, 360, 363, 381, 382, 384, 397, 403, 405, 424, 425, 426, 429, 432, 436, 437, 441, 448, 459, and 464, wherein the position is numbered with reference to SEQ ID NO:1. In some embodiments, the polynucleotide encodes an amino acid sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises an amino acid substitution, relative to SEQ ID NO:1, at one or more positions selected from A1, R7, C27, T73, A99, T100, S111, D119, Y120, Y121, H126, L128, Q151, Q165, S168, Q169, I227, S230, N245, M250, N251, A253, S260, V267, Q272, P276, H286, W289, W292, A294, N295, Q297, E301, G311, N325, N327, S333, A334, S336, S339, N341, F353, S359, A360, P363, Q381, Q382, G384, R397, G403, E405, D424, T425, S426, R429, Y432, L436, S437, Q441, Q448, T459, and P464, wherein the position is numbered with reference to SEQ ID NO:1. In some embodiments, the polynucleotide encodes an amino acid sequence that comprises one or more amino acid substitutions selected from A1V, R7S, C27Y, T73A, A99P, T100G/N, S111 N, D119P/R, Y120H, Y121R, H126E, L128H, Q151L, Q165P/R, S168T, Q169K/L/R, I227A/G/H/K/M/Q, S230P, N245T, M250G, N251D/T, A253P/T, S260K, V267E/K/L, Q272R, P276T, H286Q/S, W289C/M/S, W292A/H/P/R, A294R, N295R, Q297K/P/R/Y, E301K, G311Q, N325H, N327L, S333F, A334P, S336H/K/N/P/T, S339R/Q/W, N341V, F353I, S359D/K, A360C/K/T, P363D/H/V, Q381L, Q382R, G384T, R397H, G403T, E405G/P, D424N/Q, T425K/P/R, S426K, R429D/H/N, Y432W, L436K, S437G/P, Q441K, Q448K, T459G/K/N/R, and P464R. In some embodiments, the polynucleotide hybridizes at high stringency to the complement of SEQ ID NO:37 and encodes a cellobiohydrolase variant comprising one or more amino acid substitutions as described herein.

In some embodiments, a polynucleotide encoding a cellobiohydrolase variant encodes an amino acid sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises an amino acid substitution, relative to SEQ ID NO:1, at one or more positions selected from 2, 6, 7, 8, 12, 14, 18, 20, 21, 29, 33, 36, 37, 40, 47, 49, 50, 56, 61, 64, 67, 74, 76, 81, 83, 86, 87, 92, 94, 95, 96, 99, 100, 101, 102, 106, 107, 112, 113, 117, 118, 120, 123, 126, 128, 130, 132, 133, 139, 142, 143, 146, 151, 157, 159, 160, 161, 162, 163, 164, 165, 166, 168, 169, 176, 178, 179, 181, 206, 209, 210, 212, 213, 224, 227, 228, 230, 243, 247, 248, 249, 252, 253, 256, 259, 260, 267, 271, 272, 297, 308, 311, 312, 332, 336, 339, 340, 341, 353, 354, 356, 358, 359, 360, 363, 364, 365, 382, 384, 396, 400, 401, 404, 405, 427, 428, 436, 437, 445, 448, and 459, wherein the position is numbered with reference to SEQ ID NO:1. In some embodiments, the polynucleotide encodes an amino acid sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises an amino acid substitution, relative to SEQ ID NO:1, at one or more positions selected from P2, E6, R7, Q8, A12, W14, G18, N20, G21, A29, T33, A36, Q37, W40, N47, Q49, V50, P56, T61, R64, S67, R74, G76, S81, T83, P86, P87, V92, S94, I95, P96, A99, T100, S101, T102, S106, G107, G112, V113, A117, N118, Y120, S123, H126, L128, I130, S132, M133, A139, S142, A143, E146, Q151, V157, I159, D160, T161, L162, M163, V164, Q165, T166, S168, Q169, A176, A178, N179, P181, S206, N209, G210, A212, A213, K224, I227, E228, S230, M243, V247, T248, N249, V252, A253, S256, A259, S260, V267, K271, Q272, Q297, N308, G311, K312, A332, S336, S339, P340, N341, F353, S354, L356, N358, S359, A360, P363, A364, R365, Q382, G384, V396, A400, N401, H404, E405, A427, A428, L436, S437, E445, Q448, and T459, wherein the position is numbered with reference to SEQ ID NO:1. In some embodiments, the polynucleotide encodes an amino acid sequence that comprises one or more amino acid substitutions selected from P2H/S, E6N, R7H/S, Q8L/P, A12I, W14L, G18D, N20L/S, G21D/K, A29R/T, T33H, A36E, Q37F/H/L, W40L, N47K, Q49K, V50D/E/H/K/R, P56T, T61A, R64C, S67G, R74S, G76D, S81P, T83D, P86T, P87T, V92D/K/R/S, S94N, I95H/N, P96E/S, A99V, T100V, S101G, T102C/W, S106W/Y, G107D, G112E, V113I, A117T, N118D, Y120E/N/R, S123R/Y, H126E/L/M, L128E/H, I130V, S132I, M133F/V, A139H/T, S142E, A143M, E146L, Q151I/L, V157D/H/S, I159S, D160H, T161N/S, L162I, M163A/L, V164E/R, Q165P/T, T166R, S168G/Q/R, Q169D/R, A176G/R, A178N, N179D, P181A, S206H/K, N209S, G210A, A212C/L/N/P/R/S, A213G/H/Q, K224A/E/W, 1227A/H/K/M/T, E228G, S230P, M2431, V247A, T248S, N249D/S, V252N, A253N/P/T, S256R, A259E, S260D/K, V267L, K271A, Q272H, Q297R, N308E, G311D, K312A, A332S, S336A/E/L/N/T, S339E/L/Q/V, P340N, N341D, F353L, S354G, L356E/G/H, N358E, S359D/Y, A360D/E/Q/R/S/T/V, P363D, A364T, R365G/L, Q382A/D/H/R, G384S, V396E/R, A400V, N401D, H404N, E405P/Q, A427T, A428N/S, L436D/N, S437P, E445D, Q448T, and T459R. In some embodiments, the polynucleotide hybridizes at high stringency to the complement of SEQ ID NO:37 and encodes a cellobiohydrolase variant comprising one or more amino acid substitutions as described herein.

In still another aspect, the present invention provides expression vectors comprising a polynucleotide encoding a cellobiohydrolase variant as described herein.

In yet another aspect, the present invention provides host cells transformed with a polynucleotide or vector encoding a cellobiohydrolase variant as described herein. In some embodiments, the host cell expresses a non-naturally occurring cellobiohydrolase having the amino acid sequence of a cellobiohydrolase variant as described herein. In some embodiments, the host cell is a yeast or filamentous fungus.

In still another aspect, the present invention provides enzyme compositions comprising a recombinant cellobiohydrolase variant as described herein. In some embodiments, the enzyme composition is used in a composition for a saccharification application. In some embodiments, the enzyme composition comprising a cellobiohydrolase variant of the present invention will comprise other enzymes (e.g., one or more other cellulases).

In yet another aspect, the present invention provides methods of producing a cellobiohydrolase variant comprising culturing a host cell transformed with a polynucleotide or vector encoding a cellobiohydrolase variant as described herein under conditions sufficient for the production of the cellobiohydrolase variant by the cell. In some embodiments, the cellobiohydrolase variant polypeptide is secreted by the cell and obtained from the cell culture medium.

In still another aspect, the present invention provides methods of producing a fermentable sugar, comprising contacting a cellulosic biomass with a β-glucosidase (BGL), an endoglucanase (EG) such as a type 2 endoglucanase (EG2), a type 1 cellobiohydrolase (CBH1) such as a type 1a cellobiohydrolase (CBH1a), a glycoside hydrolase 61 protein (GH61), and a CBH2b variant as described herein under conditions in which the fermentable sugar is produced.

In yet another aspect, the present invention provides methods of producing an end-product from a cellulosic substrate, comprising (a) contacting the cellulosic substrate with a β-glucosidase (BGL), an endoglucanase (EG) such as a type 2 endoglucanase (EG2), a type 1 cellobiohydrolase (CBH1) such as a type 1a cellobiohydrolase (CBH1a), a glycoside hydrolase 61 protein (GH61), and a CBH2b variant as described herein under conditions in which fermentable sugars are produced; and (b) contacting the fermentable sugars with a microorganism in a fermentation to produce the end-product. In some embodiments, prior to step (a), the cellulosic substrate is pretreated to increase its susceptibility to hydrolysis. In some embodiments, the end-product is an alcohol, an amino acid, an organic acid, a diol, or glycerol. In some embodiments, the end-product is an alcohol (e.g., ethanol or butanol). In some embodiments, the microorganism is a yeast. In some embodiments, the process comprises a simultaneous saccharification and fermentation process. In some embodiments, the saccharification and fermentation steps are consecutive. In some embodiments, the enzyme production is simultaneous with saccharification and fermentation.

### BRIEF DESCRIPTION OF THE DRAWINGS

FIG. 1. Amino acid sequence alignment. The amino acid sequence of M. thermophila CBH2b without signal peptide (SEQ ID NO:1) (“MTCBH2B”) was aligned against 9 other proteins without signal peptides: Humicola insolens Cel6A (SEQ ID NO:5) (“2BVW”), Chaetomium thermophilum Cel6A (SEQ ID NO:6) (“AAW64927.1”), M. thermophila CBH2a (SEQ ID NO:30) (“MTCBH2A”), Humicola insolens Cel6A (SEQ ID NO:9) (“HICBH2”), Phanerochaete chtysosporium CBH2 (SEQ ID NO:24) (“PCCBH2”), Humicola insolens Cel6A (SEQ ID NO:7) (“Q9C1S9”), Trichoderma reesei CBH2 (SEQ ID NO:15) (“TRCBH2”), Chaetomium globosum CBS 148.51 unnamed protein (SEQ ID NO:8) (“XP_{—}001226029”), and Podospora anserina S mat⁺ unnamed protein (SEQ ID NO:10) (“XP_{—}001903170”). The consensus sequence of the aligned proteins is provided as SEQ ID NO:38.

FIG. 2. Amino acid sequence alignment. The amino acid sequence of M. thermophila CBH2b without signal peptide (SEQ ID NO:1) (“MTCBH2B”) was aligned against 23 other proteins without signal peptides: Trametes versicolor Cor1 (SEQ ID NO:32) (“AAF35251.1”), Lentinus sajor-caju CBH2 (SEQ ID NO:25) (“AAL15038.1”), Gibberella zeae Cel6 (SEQ ID NO:16) (“AAQ72468.1”), Volvariella volvacea CBH2-1 (SEQ ID NO:22) (“AAT64008.1”), Coniphora puteana Cel6A (SEQ ID NO:26) (“BAH59082.1”), Coniphora puteana Cel6B (SEQ ID NO:23) (“BAH59083.1”), M. thermophila CBH2a (SEQ ID NO:30) (“MTCBH2A”), Sordaria macrospora unnamed protein (SEQ ID NO:11) (“CB156846.1”), Agaricus bisporus exoglucanase 3 (SEQ ID NO:21) (“GUX3_AGABI”), Humicola insolens Cel6A (SEQ ID NO:9) (“HICBH2”), Phanerochaete chrysosporium CBH2 (SEQ ID NO:24) (“PCCBH2”), Trichoderma reesei CBH2 (SEQ ID NO:15) (“TRCBH2”), Botryotinia fuckeliana B05.10 unnamed protein (SEQ ID NO:12) (“XP_{—}001552807”), Phaeosphaeria nodorum SN15 unnamed protein (SEQ ID NO:20) (“XP_{—}001796781”), Phaeosphaeria nodorum SN15 unnamed protein (SEQ ID NO:31) (“XP_{—}001806560”), Coprinopsis cinerea okayama7#130 exocellobiohydrolase (SEQ ID NO:28) (“XP_{—}001833045”), Pyrenophora tritici-repentis Pt-1C-BFP exoglucanase-6A (SEQ ID NO:18) (“XP_{—}001933777”), Moniliophthora perniciosa FA553 unnamed protein (SEQ ID NO:29) (“XP_{—}002391276”), Verticillium albo-atrum VaMs.102 unnamed protein (SEQ ID NO:27) (“XP_{—}002999918”), Verticillium albo-atrum VaMs.102 exoglucanase (SEQ ID NO:19) (“XP_{—}003000565”), Nectria haematococca mpVI 77-13-4 unnamed protein (SEQ ID NO:13) (“XP_{—}003049522”), Magnaporthe oryzae 70-15 unnamed protein (SEQ ID NO:17) (“XP_{—}360146.1”), and Aspergillus fumigatus Af293 CBH (SEQ ID NO:14) (“XP_{—}748511.1”). The consensus sequence of the aligned proteins is provided as SEQ ID NO:39.

FIG. 3. Shake flask validation of improvements in thermostability. Variants 155 and 160 were subjected to thermo-challenge at pH 4.5, 65° C. (A) or pH 4.5, 75° C. (B) for 0-24 hours, and residual activity was determined by Avicel assay. Under both conditions, these variants were more stable than variant 81, while wild-type CBH2b was the least stable.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in analytical chemistry, cell culture, molecular genetics, organic chemistry and nucleic acid chemistry and hybridization described below are those well known and commonly employed in the art. As used herein, “a,” “an,” and “the” include plural references unless the context clearly dictates otherwise.

The terms “biomass,” “biomass substrate,” “cellulosic biomass,” “cellulosic feedstock,” and “cellulosic substrate” refer to materials that contain cellulose. Biomass can be derived from plants, animals, or microorganisms, and may include agricultural, industrial, and forestry residues, industrial and municipal wastes, and terrestrial and aquatic crops grown for energy purposes. Examples of cellulosic substrate include, but are not limited to, wood, wood pulp, paper pulp, corn fiber, corn grain, corn cobs, crop residues such as corn husks, corn stover, grasses, wheat, wheat straw, barley, barley straw, hay, rice, rice straw, switchgrass, waste paper, paper and pulp processing waste, woody or herbaceous plants, fruit or vegetable pulp, distillers grain, rice hulls, cotton, hemp, flax, sisal, sugar cane bagasse, sugar beets, sorghum, soy, switchgrass, components obtained from milling of grains, trees, branches, roots, leaves, wood chips, sawdust, shrubs and bushes, vegetables, fruits, and flowers and mixtures thereof. In some embodiments, the biomass or cellulosic substrate comprises, but is not limited to, cultivated crops (e.g., grasses, including C4 grasses, such as switchgrass, cord grass, rye grass, miscanthus, reed canary grass, or any combination thereof), sugar processing residues, for example, but not limited to, bagasse (e.g., sugar cane bagasse, beet pulp [e.g., sugar beet], or a combination thereof), agricultural residues (e.g., soybean stover, corn stover, corn fiber, rice straw, sugar cane straw, rice, rice hulls, barley straw, corn cobs, wheat straw, canola straw, oat straw, oat hulls, hemp, flax, sisal, cotton, or any combination thereof), fruit pulp, vegetable pulp, distillers' grains, and/or forestry biomass (e.g., wood, wood pulp, paper pulp, recycled wood pulp fiber, sawdust, hardwood, such as aspen wood, softwood, or a combination thereof). Furthermore, in some embodiments, the biomass or cellulosic substrate comprises cellulosic waste material and/or forestry waste materials, including but not limited to, paper and pulp processing waste, municipal paper waste, newsprint, cardboard, and the like. In some embodiments, biomass comprises one species of fiber, while in some alternative embodiments, the biomass or cellulosic substrate comprises a mixture of fibers that originate from different biomasses. In some embodiments, the biomass may also comprise transgenic plants that express ligninase and/or cellulase enzymes, see, e.g., US 2008/0104724. In some embodiments, the biomass substrate is “pretreated,” or treated using methods known in the art, such as chemical pretreatment (e.g., ammonia pretreatment, dilute acid pretreatment, dilute alkali pretreatment, or solvent exposure), physical pretreatment (e.g., steam explosion or irradiation), mechanical pretreatment (e.g., grinding or milling) and biological pretreatment (e.g., application of lignin-solubilizing microorganisms) and combinations thereof, to increase the susceptibility of cellulose to hydrolysis.

“Saccharification” refers to the process in which substrates (e.g., cellulosic biomass) are broken down via the action of cellulases to produce fermentable sugars (e.g. monosaccharides such as but not limited to glucose).

“Fermentable sugars” refers to simple sugars (monosaccharides, disaccharides and short oligosaccharides) such as but not limited to glucose, xylose, galactose, arabinose, mannose and sucrose. Fermentable sugar is any sugar that a microorganism can utilize or ferment.

As used herein, the term “fermentation” is used broadly to refer to the cultivation of a microorganism or a culture of microorganisms that use simple sugars, such as fermentable sugars, as an energy source to obtain a desired product.

As used herein, the term “cellulase” refers to a category of enzymes capable of hydrolyzing cellulose (β-1,4-glucan or β-D-glucosidic linkages) to shorter cellulose chains, oligosaccharides, cellobiose and/or glucose.

As used herein, the term “cellobiohydrolase” or “CBH” refers to a category of cellulases (EC 3.2.1.91) that hydrolyze glycosidic bonds in cellulose. In some embodiments, the cellobiohydrolase is a “type 2 cellobiohydrolase,” a cellobiohydrolase belonging to the glycoside hydrolase family 6 (GH6) family of cellulases and which is also commonly called “the Cel6 family.” Cellobiohydrolases of the GH6 family are described, for example, in the Carbohydrate Active Enzymes (CAZY) database, accessible at www.cazy.org/GH6.html.

As used herein, the term “Cl” refers to Myceliophthora thermophila, including a fungal strain described by Garg, A., 1966, “An addition to the genus Chrysosporium corda” Mycopathologia 30: 3-4. “Chrysosporium lucknowense” includes the strains described in U.S. Pat. Nos. 6,015,707, 5,811,381 and 6,573,086; US Pat. Pub. Nos. 2007/0238155, US 2008/0194005, US 2009/0099079; International Pat. Pub. Nos., WO 2008/073914 and WO 98/15633, all incorporated herein by reference, and include, without limitation, Chrysosporium lucknowense Garg 27K, VKM-F 3500 D (Accession No. VKM F-3500-D), C1 strain UV13-6 (Accession No. VKM F-3632 D), C1 strain NG7C-19 (Accession No. VKM F-3633 D), and C1 strain UV18-25 (VKM F-3631 D), all of which have been deposited at the All-Russian Collection of Microorganisms of Russian Academy of Sciences (VKM), Bakhurhina St. 8, Moscow, Russia, 113184, and any derivatives thereof. Although initially described as Chrysosporium lucknowense, C1 may currently be considered a strain of Myceliophthora thermophila. Other C1 strains and/or C1-derived strains include cells deposited under accession numbers ATCC 44006 and PTA-12255, CBS (Centraalbureau voor Schimmelcultures) 122188, CBS 251.72, CBS 143.77, CBS 272.77, CBS122190, CBS122189, and VKM F-3500D. Exemplary C1 derivatives include modified organisms in which one or more endogenous genes or sequences have been deleted or modified and/or one or more heterologous genes or sequences have been introduced. Derivatives include UV18#100f Δalpl, UV18#100f Δpyr5 Δalp1, UV18#100.f Δalp1 Δpep4 Δalp2, UV18#100.f Δpyr5 Δalp1 Δpep4 Δalp2, and UV18#100.f Δpyr4 Δpyr5 Δalp1 Δpep4 Δalp2, as described in WO2008073914 and WO2010107303, each of which is incorporated herein by reference.

As used herein, the term “wild-type M. thermophila cellobiohydrolase type 2b” or “wild-type M. thermophila CBH2b” refers to SEQ ID NO:1, the mature peptide sequence (i.e., lacking a signal peptide) of cellobiohydrolase type 2b that is expressed by the naturally occurring fungal strain M. thermophila.

As used herein, the term “variant” refers to a cellobiohydrolase polypeptide or polynucleotide encoding a cellobiohydrolase polypeptide comprising one or more modifications relative to wild-type M. thermophila CBH2b or the wild-type polynucleotide encoding M. thermophila CBH2b such as substitutions, insertions, deletions, and/or truncations of one or more amino acid residues or of one or more specific nucleotides or codons in the polypeptide or polynucleotide, respectively.

As used herein, “cellobiohydrolase polypeptide” refers to a polypeptide having cellobiohydrolase activity.

As used herein, the term “cellobiohydrolase polynucleotide” refers to a polynucleotide encoding a polypeptide having cellobiohydrolase activity.

The terms “improved” or “improved properties,” as used in the context of describing the properties of a cellobiohydrolase variant, refers to a cellobiohydrolase variant polypeptide that exhibits an improvement in any property as compared to the wild-type M. thermophila CBH2b (SEQ ID NO:1). Improved properties may include increased protein expression, increased thermoactivity, increased thermostability, increased pH activity, increased stability (e.g., increased pH stability), increased product specificity, increased specific activity, increased substrate specificity, increased resistance to substrate or end-product inhibition, increased chemical stability, reduced inhibition by glucose, increased resistance to inhibitors (e.g., acetic acid, lectins, tannic acids, and phenolic compounds) and altered pH/temperature profile.

As used herein, the phrase “improved thermoactivity” or “increased thermoactivity” refers to a variant enzyme displaying an increase, relative to a reference enzyme (e.g., a wild-type cellobiohydrolase), in the amount of cellobiohydrolase enzymatic activity (e.g., substrate hydrolysis) in a specified time under specified reaction conditions, for example, elevated temperature. Exemplary methods for measuring cellobiohydrolase activity are provided in the Examples and include, but are not limited to, measuring cellobiose production from crystalline cellulose as measured by colorimetric assay or HPLC. To compare cellobiohydrolase activity of two recombinantly expressed proteins, the specific activity (activity per mole enzyme or activity per gram enzyme) can be compared. Alternatively, cells expressing and secreting the recombinant proteins can be cultured under the same conditions and the cellobiohydrolase activity per volume culture medium can be compared.

As used herein, the phrase “improved thermostability” or “increased thermostability” refers to a variant enzyme displaying an increase in “residual activity” relative to a reference enzyme (e.g., a wild-type cellobiohydrolase). Residual activity is determined by (1) exposing the variant enzyme or wild-type enzyme to stress conditions of elevated temperature, optionally at lowered pH, for a period of time and then determining cellobiohydrolase activity; (2) exposing the variant enzyme or wild-type enzyme to unstressed conditions for the same period of time and then determining cellobiohydrolase activity; and (3) calculating residual activity as the ratio of activity obtained under stress conditions (1) over the activity obtained under unstressed conditions (2). For example, the cellobiohydrolase activity of the enzyme exposed to stress conditions (“a”) is compared to that of a control in which the enzyme is not exposed to the stress conditions (“b”), and residual activity is equal to the ratio a/b. A variant with increased thermostability will have greater residual activity than the reference enzyme (e.g., a wild-type cellobiohydrolase). In one embodiment the enzymes are exposed to stress conditions of 67° C. at pH 4.5 for 1 hr, but other cultivation conditions, such as conditions described herein, can be used.

As used herein, the phrase “improved stability” or “increased stability” refers to a variant enzyme that retains substantially all of its residual activity under stressed conditions relative to its activity under unstressed conditions. In some embodiments, a stressed condition is elevated temperature, lowered temperature, elevated pH, lowered pH, elevated salt concentration, lowered salt concentration, or increased concentration of an enzyme inhibitor (e.g., acetic acid, lectins, tannic acids, and phenolic compounds). Residual activity is determined by (1) exposing the variant enzyme to stress conditions, such as elevated temperature or lowered pH, for a period of time and then determining cellobiohydrolase activity; (2) exposing the variant enzyme to unstressed conditions for the same period of time and then determining cellobiohydrolase activity; and (3) calculating residual activity as the ratio of activity obtained under stress conditions (1) over the activity obtained under unstressed conditions (2). A variant with increased stability will have greater residual activity than a reference enzyme exposed to the same stressed conditions (e.g., a wild-type cellobiohydrolase). In one embodiment the enzymes are exposed to stress conditions of 67° C. at pH 4.5 for 1 hr, but other cultivation conditions, such as conditions described herein, can be used.

As used herein, the term “reference enzyme” refers to an enzyme to which a variant enzyme of the present invention is compared in order to determine the presence of an improved property in the variant enzyme being evaluated, including but not limited to improved thermoactivity, improved thermostability, or improved stability. In some embodiments, a reference enzyme is a wild-type enzyme (e.g., wild-type M. thermophila CBH2b). In some embodiments, a reference enzyme is another variant enzyme (e.g., another variant enzyme of the present invention).

As used herein, “polynucleotide” refers to a polymer of deoxyribonucleotides or ribonucleotides in either single- or double-stranded form, and complements thereof.

Nucleic acids “hybridize” when they associate, typically in solution. Nucleic acids hybridize due to a variety of well-characterized physico-chemical forces, such as hydrogen bonding, solvent exclusion, base stacking and the like. As used herein, the term “stringent hybridization wash conditions” in the context of nucleic acid hybridization experiments, such as Southern and Northern hybridizations, are sequence dependent, and are different under different environmental parameters. An extensive guide to the hybridization of nucleic acids is found in Tijssen, 1993, “Laboratory Techniques in Biochemistry and Molecular Biology-Hybridization with Nucleic Acid Probes,” Part I, Chapter 2 (Elsevier, New York), which is incorporated herein by reference. For polynucleotides of at least 100 nucleotides in length, low to very high stringency conditions are defined as follows: prehybridization and hybridization at 42° C. in 5×SSPE, 0.3% SDS, 200 μg/ml sheared and denatured salmon sperm DNA, and either 25% formamide for low stringencies, 35% formamide for medium and medium-high stringencies, or 50% formamide for high and very high stringencies, following standard Southern blotting procedures. For polynucleotides of at least 100 nucleotides in length, the carrier material is finally washed three times each for 15 minutes using 2×SSC, 0.2% SDS 50° C. (low stringency), at 55° C. (medium stringency), at 60° C. (medium-high stringency), at 65° C. (high stringency), or at 70° C. (very high stringency).

The terms “peptide,” “polypeptide,” and “protein” are used interchangeably herein to refer to a polymer of amino acid residues.

The term “amino acid” refers to naturally occurring and synthetic amino acids, as well as amino acid analogs. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

A “conservative substitution,” as used with respect to amino acids, refers to the substitution of an amino acid with a chemically similar amino acid. Amino acid substitutions which often preserve the structural and/or functional properties of the polypeptide in which the substitution is made are known in the art and are described, for example, by H. Neurath and R. L. Hill, 1979, in “The Proteins,” Academic Press, New York. The most commonly occurring exchanges are isoleucine/valine, tyrosine/phenylalanine, aspartic acid/glutamic acid, lysine/arginine, methionine/leucine, aspartic acid/asparagine, glutamic acid/glutamine, leucine/isoleucine, methionine/isoleucine, threonine/serine, tryptophan/phenylalanine, tyrosine/histidine, tyrosine/tryptophan, glutamine/arginine, histidine/asparagine, histidine/glutamine, lysine/asparagine, lysine/glutamine, lysine/glutamic acid, phenylalanine/leucine, phenylalanine/methionine, serine/alanine, serine/asparagine, valine/leucine, and valine/methionine. In some embodiments, there may be at least 1, at least 2, at least 3, at least 4, at least 5, at least 6, at least 7, at least 8, at least 9, at least 10, at least 15, at least 20, at least 25, at least 30, at least 35, or at least 40 conservative substitutions.

The following nomenclature may be used to describe substitutions in a reference sequence relative to a reference sequence or a variant polypeptide or nucleic acid sequence: “R-#-V,” where # refers to the position in the reference sequence, R refers to the amino acid (or base) at that position in the reference sequence, and V refers to the amino acid (or base) at that position in the variant sequence. In some embodiments, an amino acid (or base) may be called “X,” by which is meant any amino acid (or base). As a non-limiting example, for a variant polypeptide described with reference to SEQ ID NO:1, “Y120H” indicates that in the variant polypeptide, the tyrosine at position 120 of the reference sequence is replaced by histidine, with amino acid position being determined by optimal alignment of the variant sequence with SEQ ID NO:1. Similarly, “Y120H/R” describes two variants: a variant in which the tyrosine at position 120 of the reference sequence is replaced by histidine and a variant in which the amino acid at position 120 of the reference sequence is replaced by arginine.

The term “amino acid substitution set” or “substitution set” refers to a group of amino acid substitutions. A substitution set can have 1, 2, 3, 4, 5, 6, 7, 8, 9, 10, 11, 12, 13, 14, 15, or more amino acid substitutions. In some embodiments, a substitution set refers to the set of amino acid substitutions that is present in any of the variant cellobiohydrolases listed in Table 3 (i.e., in any of Tables 3a, 3b, 3c, and/or 3d), Table 4 (i.e., in any of Tables 4a, 4b, 4c, and/or 4d), and/or Table 6. For example, the substitution set for Variant 77 (Table 3b) consists of the amino acid substitutions D160P, S230P, A253P, and A334P.

The term “isolated” refers to a nucleic acid, polynucleotide, polypeptide, protein, or other component that is partially or completely separated from components with which it is normally associated (other proteins, nucleic acids, cells, etc.). In some embodiments, an isolated polypeptide or protein is a recombinant polypeptide or protein.

A nucleic acid (such as a polynucleotide), a polypeptide, or a cell is “recombinant” when it is artificial or engineered, or derived from or contains an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Identity” or “percent identity,” in the context of two or more polypeptide sequences, refers to two or more sequences or subsequences that are the same or have a specified percentage of amino acid residues that are the same (e.g., share at least about 70%, at least about 75%, at least about 80%, at least about 85%, at least about 88% identity, at least about 89%, at least about 90%, at least about 91%, at least about 92%, at least about 93%, at least about 94%, at least about 95%, at least about 96%, at least about 97%, at least about 98%, or at least about 99% identity) over a specified region to a reference sequence, when compared and aligned for maximum correspondence over a comparison window, or designated region as measured using a sequence comparison algorithms or by manual alignment and visual inspection.

Optimal alignment of sequences for comparison and determination of sequence identity can be determined by a sequence comparison algorithm or by visual inspection (see, generally, Ausubel et al., infra). When optimally aligning sequences and determining sequence identity by visual inspection, percent sequence identity is calculated as the number of residues of the test sequence that are identical to the reference sequence divided by the number of non-gap positions and multiplied by 100. When using a sequence comparison algorithm, test and reference sequences are entered into a computer, subsequence coordinates and sequence algorithm program parameters are designated. The sequence comparison algorithm then calculates the percent sequence identities for the test sequences relative to the reference sequence, based on the program parameters.

An algorithm that may be used to determine whether a variant cellobiohydrolase has sequence identity to SEQ ID NO:1 is the BLAST algorithm, which is described in Altschul et al., 1990, J. Mol. Biol. 215:403-410, which is incorporated herein by reference. Software for performing BLAST analyses is publicly available through the National Center for Biotechnology Information (on the worldwide web at ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. For amino acid sequences, the BLASTP program uses as defaults a word size (W) of 3, an expectation (E) of 10, and the BLOSUM62 scoring matrix (see Henikoff & Henikoff, 1989, Proc. Natl. Acad. Sci. USA 89:10915). Other programs that may be used include the Needleman-Wunsch procedure, J. Mol. Biol. 48: 443-453 (1970), using blosum62, a Gap start penalty of 7 and gap extend penalty of 1; and gapped BLAST 2.0 (see Altschul, et al. 1997, Nucleic Acids Res., 25:3389-3402), both available to the public at the National Center for Biotechnology Information Website.

Multiple sequences can be aligned with each other by visual inspection or using a sequence comparison algorithm, such as PSI-BLAST (Altschul, et al., 1997, supra) or “T-Coffee” (Notredame et al., 2000, J. Mol. Bio. 302:205-17). T-Coffee alignments may be carried out using default parameters (T-Coffee Technical Documentation, Version 8.01, July 2009, WorldWideWeb.tcoffee.org), or Protein Align. In Protein Align, alignments are computed by optimizing a function based on residue similarity scores (obtained from applying an amino acid substitution matrix to pairs of aligned residues) and gap penalties. Penalties are imposed for introducing an extending gaps in one sequence with respect to another. The final optimized function value is referred to as the alignment score. When aligning multiple sequences, Protein Align optimizes the “sum of pairs” score, i.e., the sum of all the separate pairwise alignment scores.

The phrase “substantial sequence identity” or “substantial identity,” in the context of two nucleic acid or polypeptide sequences, refers to a sequence that has at least 70% identity to a reference sequence. Percent identity can be any integer from 70% to 100%. Two nucleic acid or polypeptide sequences that have 100% sequence identity are said to be “identical.” A nucleic acid or polypeptide sequence are said to have “substantial sequence identity” to a reference sequence when the sequences have at least about 70%, at least about 75%, at least 80%, at least 85%, at least 90%, at least 91%, at least 92%, at least 93%, at least 94%, at least 95%, at least 96%, at least 97%, at least 98%, or at least 99% or greater sequence identity as determined using the methods described herein, such as BLAST using standard parameters as described above. For example, for an alignment that extends along the entire length of SEQ ID NO:1, there may be at least 326, at least 349, at least 372, at least 396, at least 419, at least 424, at least 428, at least 433, at least 438, at least 442, at least 447, at least 452, at least 456, or at least 461 amino acids identical between a variant sequence and SEQ ID NO:1.

The term “pre-protein” refers to a protein including an amino-terminal signal peptide (or leader sequence) region attached. The signal peptide is cleaved from the pre-protein by a signal peptidase prior to secretion to result in the “mature” or “secreted” protein.

A “vector” is a DNA construct for introducing a DNA sequence into a cell. A vector may be an expression vector that is operably linked to a suitable control sequence capable of effecting the expression in a suitable host of the polypeptide encoded in the DNA sequence. An “expression vector” has a promoter sequence operably linked to the DNA sequence (e.g., transgene) to drive expression in a host cell, and in some embodiments a transcription terminator sequence.

The term “operably linked” refers to a configuration in which a control sequence is appropriately placed at a position relative to the coding sequence of the DNA sequence such that the control sequence influences the expression of a polypeptide.

An amino acid or nucleotide sequence (e.g., a promoter sequence, signal peptide, terminator sequence, etc.) is “heterologous” to another sequence with which it is operably linked if the two sequences are not associated in nature.

The terms “transform” or “transformation,” as used in reference to a cell, means a cell has a non-native nucleic acid sequence integrated into its genome or as an episome (e.g., plasmid) that is maintained through multiple generations.

The term “introduced,” as used in the context of inserting a nucleic acid sequence into a cell, means conjugated, transfected, transduced or transformed (collectively “transformed”) or otherwise incorporated into the genome of, or maintained as an episome in, the cell.

### DETAILED DESCRIPTION OF THE INVENTION

### I. Introduction

Fungi, bacteria, and other organisms produce a variety of cellulases and other enzymes that act in concert to catalyze decrystallization and hydrolysis of cellulose to yield fermentable sugars. One such fungus is M. thermophila, which was described by Garg, 1966, “An addition to the genus Chrysosporium corda” Mycopathologia 30: 3-4; see also U.S. Pat. Nos. 6,015,707 and 6,573,086, which are incorporated herein by reference for all purposes.

The cellobiohydrolase variants described herein are particularly useful for the production of fermentable sugars from cellulosic biomass. In one aspect, the present invention relates to cellobiohydrolase variants that have improved properties, relative to wild-type M. thermophila cellobiohydrolase, under process conditions used for saccharification of biomass. Exemplary properties include increased thermostability and/or increased thermoactivity and/or increased pH tolerance. In another aspect, the present invention relates to methods of generating fermentable sugars from cellulosic biomass, by contacting the biomass with a cellulase composition comprising a cellobiohydrolase variant as described herein under conditions suitable for the production of fermentable sugars.

Various aspects of the invention are described in the following sections.

### II. Cellobiohydrolase Type 2 Variants

### Properties of Cellobiohydrolase Variants

In one aspect, the present invention provides CBH2b variants having improved properties over a wild-type cellobiohydrolase. In some embodiments, the CBH2b variants of the present invention exhibit increased thermostability and/or increased thermoactivity in comparison to a wild-type CBH2b (e.g., a M. thermophila CBH2b having the amino acid sequence of SEQ ID NO:1) under conditions relevant to commercial cellulose hydrolysis processes.

In some embodiments, the present invention provides a recombinant M. thermophila CBH2b variant comprising at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:1 and comprising an amino acid substitution, relative to SEQ ID NO:1, at one or more positions selected from A1, R7, C27, T73, A99, T100, S111, D119, Y120, Y121, H126, L128, Q151, Q165, S168, Q169, I227, S230, N245, M250, N251, A253, S260, V267, Q272, P276, H286, W289, W292, A294, N295, Q297, E301, G311, N325, N327, S333, A334, S336, S339, N341, F353, S359, A360, P363, Q381, Q382, G384, R397, G403, E405, D424, T425, S426, R429, Y432, L436, S437, Q441, Q448, T459, and P464, wherein the position is numbered with reference to the amino acid sequence of SEQ ID NO:1, and wherein the variant has increased thermostability and/or thermoactivity in comparison to wild-type M. thermophila CBH2b (SEQ ID NO:1). In some embodiments, a CBH2b variant of the present invention has an amino acid sequence that is encoded by a nucleic acid that hybridizes under stringent conditions to the complement of SEQ ID NO:37 (e.g., over substantially the entire length of a nucleic acid exactly complementary to SEQ ID NO:37) and comprises an amino acid substitution, relative to SEQ ID NO:1, at one or more positions selected from A1, R7, C27, T73, A99, T100, S111, D119, Y120, Y121, H126, L128, Q151, Q165, S168, Q169, I227, S230, N245, M250, N251, A253, S260, V267, Q272, P276, H286, W289, W292, A294, N295, Q297, E301, G311, N325, N327, S333, A334, S336, S339, N341, F353, S359, A360, P363, Q381, Q382, G384, R397, G403, E405, D424, T425, S426, R429, Y432, L436, S437, Q441, Q448, T459, and P464, wherein the position is numbered with reference to the amino acid sequence of SEQ ID NO:1.

In some embodiments, the present invention provides a recombinant M. thermophila CBH2b variant comprising at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:1 and comprising one or more amino acid substitutions selected from A1V, R7S, C27Y, T73A, A99P, T100G/N, S111N, D119P/R, Y120H, Y121R, H126E, L128H, Q151L, Q165P/R, S168T, Q169K/L/R, I227A/G/H/K/M/Q, S230P, N245T, M250G, N251D/T, A253P/T, S260K, V267E/K/L, Q272R, P276T, H286Q/S, W289C/M/S, W292A/H/P/R, A294R, N295R, Q297K/P/R/V, E301K, G311Q, N325H, N327L, S333F, A334P, S336H/K/N/P/T, S339R/Q/W, N341V, F353I, S359D/K, A360C/K/T, P363D/H/V, Q381L, Q382R, G384T, R397H, G403T, E405G/P, D424N/Q, T425K/P/R, S426K, R429D/H/N, Y432W, L436K, S437G/P, Q441K, Q448K, T459G/K/N/R, and P464R, wherein the residue is numbered with reference to the amino acid sequence of SEQ ID NO:1, and wherein the variant has increased thermostability and/or thermoactivity in comparison to wild-type M. thermophila CBH2b (SEQ ID NO:1). In some embodiments, a CBH2b variant of the present invention has an amino acid sequence that is encoded by a nucleic acid that hybridizes under stringent conditions to the complement of SEQ ID NO:37 (e.g., over substantially the entire length of a nucleic acid exactly complementary to SEQ ID NO:37) and comprises one or more amino acid substitutions selected from A1V, R7S, C27Y, T73A, A99P, T100G/N, S111N, D119P/R, Y120H, Y121R, H126E, L128H, Q151L, Q165P/R, S168T, Q169K/L/R, I227A/G/H/K/M/Q, S230P, N245T, M250G, N251D/T, A253P/T, S260K, V267E/K/L, Q272R, P276T, H286Q/S, W289C/M/S, W292A/H/P/R, A294R, N295R, Q297K/P/R/V, E301K, G311Q, N325H, N327L, S333F, A334P, S336H/K/N/P/T, S339R/Q/W, N341V, F353I, S359D/K, A360C/K/T, P363D/H/V, Q381L, Q382R, G384T, R397H, G403T, E405G/P, D424N/Q, T425K/P/R, S426K, R429D/H/N, Y432W, L436K, S437G/P, Q441K, Q448K, T459G/K/N/R, and P464R.

In some embodiments, the M. thermophila CBH2b variant of the present invention exhibits at least about a 1.1 fold, at least about a 1.5 fold, at least about a 2.0 fold, at least about a 2.5 fold, at least about a 3.0 fold, at least about a 3.5 fold, at least about a 4.0 fold, at least about a 4.5 fold, at least about a 5.0 fold increase or more in thermostability relative to wild-type M. thermophila CBH2b (SEQ ID NO:1), as identified in Table 3 (i.e., in any of Tables 3a, 3b, 3c, and/or 3d) or Table 4 (i.e., in any of Tables 4a, 4b, 4c, and/or 4d), wherein fold improvement in thermostability is measured as described in the Examples (i.e., expressed in S. cerevisiae).

In some embodiments, the M. thermophila CBH2b variant comprises at least 1, at least 2, at least 3, at least 4, at least 5, at least 6, at least 7, at least 8, at least 9, at least 10 or more amino acid residues which have been substituted (e.g., with substitutions described herein) as compared to the amino acid sequence of the wild-type cellobiohydrolase protein from which the cellobiohydrolase variant is derived. In some embodiments, the M. thermophila CBH2b variant differs from the CBH2b of SEQ ID NO:1 at no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, or no more than 5 residues.

In some embodiments, a M. thermophila CBH2b variant of the present invention comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises one or more amino acid substitution sets selected from the substitution sets identified in Table 3 (i.e., in any of Tables 3a, 3b, 3c, and/or 3d). In some embodiments, a M. thermophila CBH2b variant of the present invention comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1, comprises one or more amino acid substitution sets selected from the substitution sets identified in Table 3 (i.e., in any of Tables 3a, 3b, 3c, and/or 3d), and further comprises at least 1, 2, 3, 4, 5, 6, 7, 8, 9, 10, or more additional amino acid substitutions. In some embodiments, a M. thermophila CBH2b variant of the present invention comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises an amino acid substitution set selected from the substitution sets showing at least 1.1 to 1.9 fold, at least 2.0 to 2.9 fold, at least 3.0 or higher improvement in thermostability over the M. thermophila wild-type CBH2b (SEQ ID NO:1), as identified in Table 3 (i.e., in any of Tables 3a, 3b, 3c, and/or 3d).

In some embodiments, a M. thermophila CBH2b variant of the present invention comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises one or more amino acid substitution sets selected from the substitution sets identified in Table 4 (i.e., in any of Tables 4a, 4b, 4c, and/or 4d). In some embodiments, a M. thermophila CBH2b variant of the present invention comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1, comprises one or more amino acid substitution sets selected from the substitution sets identified in Table 4 (i.e., in any of Tables 4a, 4b, 4c, and/or 4d), and further comprises at least 1, 2, 3, 4, 5, 6, 7, 8, 9, 10, or more additional amino acid substitutions. In some embodiments, a M. thermophila CBH2b variant of the present invention comprises at least about 70% (or at least about 75%, at least about 80%, at least about 85%, at least about 90%, at least about 95%, at least about 96%, at least about 97%, or at least about 98%) sequence identity to SEQ ID NO:1 and comprises an amino acid substitution set selected from the substitution sets showing at least 1.1 to 1.9 fold, at least 2.0 to 2.9 fold, at least 3.0 or higher improvement in thermostability over the cellobiohydrolase variant 81 (SEQ ID NO:2), as identified in Table 4 (i.e., in any of Tables 4a, 4b, 4c, and/or 4d).

In some embodiments, the present invention encompasses any of the cellobiohydrolase proteins in Tables 3-4, as well as any variants that comprise an amino acid substitution set provided in Table 3 (i.e., in any of Tables 3a, 3b, 3c, and/or 3d) or Table 4 (i.e., in any of Tables 4a, 4b, 4c, and/or 4d) and comprise at least 70% (or at least 75%, at least 80%, at least 85%, at least 90%, at least 91%, at least 92%, at least 93%, at least 94%, at least 95%, at least 96%, at least 97%, at least 98%, or at least 99%) sequence identity to the wild-type M. thermophila CBH2b (SEQ ID NO:1).

Certain cellobiohydrolase variants comprise an amino acid substitution, relative to SEQ ID NO:1, at one or more positions selected from S230, A253, E405, and S437. In some embodiments, a M. thermophila cellobiohydrolase variant of the present invention comprises one or more amino acid substitutions selected from S230P, A253P, E405P, and S437P. In some embodiments, a M. thermophila cellobiohydrolase variant of the present invention comprises the amino acid substitutions of variant 81, i.e., the amino acid substitutions S230P, A253P, E405P, and S437P. In some embodiments, the M. thermophila cellobiohydrolase variant has the amino acid sequence of SEQ ID NO:2.

Certain cellobiohydrolase variants comprise an amino acid substitution, relative to SEQ ID NO:1, at one or more positions selected from R7, T100, Y120, Q165, S230, A253, S339, E405, S437P, and T459. In some embodiments, a M. thermophila cellobiohydrolase variant of the present invention comprises one or more amino acid substitutions selected from R7S, T100G, Y120H, Q165R, S230P, A253P, S339Q, E405P, S437P, and T459N. In some embodiments, a M. thermophila cellobiohydrolase variant of the present invention comprises the amino acid substitutions of variant 160, i.e., the amino acid substitutions R7S, T100G, Y120H, Q165R, S230P, A253P, S339Q, E405P, S437P, and T459N. In some embodiments, the M. thermophila cellobiohydrolase variant has the amino acid sequence of SEQ ID NO:3.

Certain cellobiohydrolase variants comprise an amino acid substitution, relative to SEQ ID NO:1, at one or more positions selected from R7, T100, Y120, Q165, I227, S230, A253, S339, E405, S437, and T459. In some embodiments, a M. thermophila cellobiohydrolase variant of the present invention comprises one or more amino acid substitutions selected from R7S, T100G, Y120H, Q165R, I227M, S230P, A253P, S339Q, E405P, S437P, and T459N. In some embodiments, a M. thermophila cellobiohydrolase variant of the present invention comprises the amino acid substitutions of variant 155, i.e., the amino acid substitutions R7S, T100G, Y120H, Q165R, I227M, S230P, A253P, S339Q, E405P, S437P, and T459N. In some embodiments, the M. thermophila cellobiohydrolase variant has the amino acid sequence of SEQ ID NO:4.

In some embodiments, the present invention provides a recombinant M. thermophila CBH2b variant comprising at least 70% (or at least 75%, at least 80%, at least 85%, at least 90%, at least 91%, at least 92%, at least 93%, at least 94%, at least 95%, at least 96%, at least 97%, at least 98%, or at least 99%) sequence identity to the wild-type M. thermophila cellobiohydrolase type 2b of SEQ ID NO:1 and comprising one or more pairs of amino acid substitutions selected from P109C and A279C, A129C and Q451C, I159C and A221C, V247C and A299C, A304C and A360C, L128C and W449C, A284C and L319C, I219C and A269C, I207C and T261C, A300C and L356C, and V267C and D309C, wherein the position is numbered with reference to SEQ ID NO:1. Without being bound to a particular theory, it is believed that introducing cysteine mutations in the amino acid sequence of M. thermophila cellobiohydrolase results in the formation of disulfide bonds that enhance the stability of the M. thermophila cellobiohydrolase protein. In some embodiments, a M. thermophila cellobiohydrolase variant of the present invention comprises a pair of amino acid substitutions at P109C and A279C. In some embodiments, a M. thermophila cellobiohydrolase variant of the present invention comprises a pair of amino acid substitutions at A129C and Q451C. In some embodiments, a M. thermophila cellobiohydrolase variant of the present invention comprises a pair of amino acid substitutions at I159C and A221C. In some embodiments, a M. thermophila cellobiohydrolase variant of the present invention comprises a pair of amino acid substitutions at V247C and A299C. In some embodiments, a M. thermophila cellobiohydrolase variant of the present invention comprises a pair of amino acid substitutions at A304C and A360C. In some embodiments, a M. thermophila cellobiohydrolase variant of the present invention comprises a pair of amino acid substitutions at L128C and W449C. In some embodiments, a M. thermophila cellobiohydrolase variant of the present invention comprises a pair of amino acid substitutions at A284C and L319C. In some embodiments, a M. thermophila cellobiohydrolase variant of the present invention comprises a pair of amino acid substitutions at I219C and A269C. In some embodiments, a M. thermophila cellobiohydrolase variant of the present invention comprises a pair of amino acid substitutions at I207C and T261C. In some embodiments, a M. thermophila cellobiohydrolase variant of the present invention comprises a pair of amino acid substitutions at A300C and L356C. In some embodiments, a M. thermophila cellobiohydrolase variant of the present invention comprises a pair of amino acid substitutions at V267C and D309C. In some embodiments, a M. thermophila cellobiohydrolase variant of the present invention comprises two pairs of amino acid substitutions at A300C and L356C and at A304C and A360C. In some embodiments, a M. thermophila cellobiohydrolase variant of the present invention comprises two pairs of amino acid substitutions at I159C and A221C and at A304C and A360C. In some embodiments, a M. thermophila cellobiohydrolase variant of the present invention comprises two pairs of amino acid substitutions at I159C and A221C and at A300C and L356C. In some embodiments, the M. thermophila cellobiohydrolase variant comprising one or more pairs of amino acid substitutions as described herein exhibits at least about a 1.1 fold, at least about a 1.5 fold, at least about a 2.0 fold, at least about a 2.5 fold, at least about a 3.0 fold, at least about a 3.5 fold, at least about a 4.0 fold, at least about a 4.5 fold, at least about a 5.0 fold increase or more in thermostability relative to wild-type M. thermophila CBH2b (SEQ ID NO:1).

In some embodiments, the present invention provides a recombinant M. thermophila CBH2b variant comprising at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprising an amino acid substitution, relative to SEQ ID NO:1, at one or more positions selected from P2, E6, R7, Q8, A12, W14, G18, N20, G21, A29, T33, A36, Q37, W40, N47, Q49, V50, P56, T61, R64, S67, R74, G76, S81, T83, P86, P87, V92, S94, 195, P96, A99, T100, S101, T102, S106, G107, G112, V113, A117, N118, Y120, S123, H126, L128, I130, S132, M133, A139, S142, A143, E146, Q151, V157, I159, D160, T161, L162, M163, V164, Q165, T166, S168, Q169, A176, A178, N179, P181, S206, N209, G210, A212, A213, K224, I227, E228, S230, M243, V247, T248, N249, V252, A253, S256, A259, S260, V267, K271, Q272, Q297, N308, G311, K312, A332, S336, S339, P340, N341, F353, S354, L356, N358, S359, A360, P363, A364, R365, Q382, G384, V396, A400, N401, H404, E405, A427, A428, L436, S437, E445, Q448, and T459, wherein the position is numbered with reference to the amino acid sequence of SEQ ID NO:1, and wherein the variant has increased thermoactivity and/or thermostability in comparison to wild-type M. thermophila CBH2b (SEQ ID NO:1).

In some embodiments, the present invention provides a recombinant M. thermophila CBH2b variant comprising at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprising one or more amino acid substitutions selected from P2H/S, E6N, R7H/S, Q8L/P, A12I, W14L, G18D, N20L/S, G21D/K, A29R/T, T33H, A36E, Q37F/H/L, W40L, N47K, Q49K, V50D/E/H/K/R, P56T, T61A, R64C, S67G, R74S, G76D, S81P, T83D, P86T, P87T, V92D/K/R/S, S94N, I95H/N, P96E/S, A99V, T100V, S101G, T102C/W, S106W/Y, G107D, G112E, V113I, A117T, N118D, Y120E/N/R, S123RN, H126E/L/M, L128E/H, I130V, S1321, M133F/V, A139H/T, S142E, A143M, E146L, Q151I/L, V157D/H/S, I159S, D160H, T161N/S, L162I, M163A/L, V164E/R, Q165P/T, T166R, S168G/Q/R, Q169D/R, A176G/R, A178N, N179D, P181A, S206H/K, N209S, G210A, A212C/L/N/P/R/S, A213G/H/Q, K224A/E/W, I227A/H/K/M/T, E228G, S230P, M243I, V247A, T248S, N249D/S, V252N, A253N/P/T, S256R, A259E, S260D/K, V267L, K271A, Q272H, Q297R, N308E, G311D, K312A, A332S, S336A/E/L/N/T, S339E/L/Q/V, P340N, N341D, F353L, S354G, L356E/G/H, N358E, S359D/Y, A360D/E/Q/R/S/T/V, P363D, A364T, R365G/L, Q382A/D/H/R, G384S, V396E/R, A400V, N401D, H404N, E405P/Q, A427T, A428N/S, L436D/N, S437P, E445D, Q448T, and T459R, wherein the residue is numbered with reference to the amino acid sequence of SEQ ID NO:1, and wherein the variant has increased thermoactivity and/or thermostability in comparison to wild-type M. thermophila CBH2b (SEQ ID NO:1).

In some embodiments, a M. thermophila CBH2b variant of the present invention exhibits from up to about 1.2-fold improvement, or from about 1.2-fold to about 1.4-fold improvement, or greater than 1.4-fold improvement in glucose production using β-glucosidase relative to wild-type M. thermophila CBH2b (SEQ ID NO:1) as identified in Table 6, wherein improvement in glucose production is measured as described in Example 12. In some embodiments, a M. thermophila CBH2b variant of the present invention exhibits about a 5%, or 10%, or greater, improvement in glucose production using β-glucosidase relative to wild-type M. thermophila CBH2b (SEQ ID NO:1).

In some embodiments, the M. thermophila CBH2b variant comprises at least 1, at least 2, at least 3, at least 4, at least 5, at least 6, at least 7, at least 8, at least 9, at least 10 or more amino acid residues which have been substituted (e.g., with substitutions described herein) as compared to the amino acid sequence of the wild-type cellobiohydrolase protein from which the cellobiohydrolase variant is derived.

In some embodiments, the present invention encompasses any of the cellobiohydrolase proteins in Table 6, as well as any variants that comprise an amino acid substitution set provided in Table 6 and comprise at least 70% (or at least 75%, at least 80%, at least 85%, at least 90%, at least 91%, at least 92%, at least 93%, at least 94%, at least 95%, at least 96%, at least 97%, at least 98%, or at least 99%) sequence identity to the wild-type M. thermophila CBH2b (SEQ ID NO:1).

In some embodiments, a M. thermophila CBH2b variant of the present invention comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1 and comprises one or more amino acid substitution sets selected from the substitution sets set forth in Table 6. In some embodiments, a M. thermophila CBH2b variant of the present invention comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1, comprises one or more amino acid substitution sets selected from the substitution sets identified in Table 6, and further comprises at least 1, 2, 3, 4, 5, 6, 7, 8, 9, 10, or more additional amino acid substitutions. In some embodiments, a M. thermophila CBH2b variant of the present invention comprises an amino substitution set selected from the substitution sets set forth in Table 6 and at least one amino acid substitution set forth in Table 3 (i.e., in any of Tables 3a, 3b, 3c, and/or 3d) or Table 4 (i.e., in any of Tables 4a, 4b, 4c, and/or 4d). In some embodiments a M. thermophila CBH2b variant of the present invention comprises an amino acid substitution set selected from the substitution sets set forth in Table 3 (i.e., in any of Tables 3a, 3b, 3c, and/or 3d) or Table 4 (i.e., in any of Tables 4a, 4b, 4c, and/or 4d) and at least one amino acid substitution set forth in Table 6.

Certain cellobiohydrolase variants comprise an amino acid substitution at one or more positions selected from H126, L128, Q165, Q169, I227, S339, S359, and A360. In some embodiments, a M. thermophila CBH2b variant of the present invention comprises one or more amino acid substitutions selected from H126M, L128E/H, Q165P, Q169R, I227K, S339E/Q, S359D, and A360D. Certain cellobiohydrolase variants further comprise an amino acid substitution at one or more positions selected from R64, P86, P87, T102, S206, A212, S230, A253, V267, K271, G311, A332, S336, P340, Q382, and R429. In some embodiments, a M. thermophila CBH2b variant of the present invention comprises one or more amino acid substitutions selected from R64c, P87T, T102C, S206H/K, A212C/L/N/P/R/S, S230P, A253T, V267L, K271A, G311Q, A332S, S336N, P340N, Q382D, and R429N.

In some embodiments, the present invention relates to a method of making M. thermophila CBH2b variants having improved thermostability and/or improved thermoactivity. In some embodiments, the method comprises:
(a) identifying a sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1;
(b) aligning the identified sequence with the sequence of SEQ ID NO:1; and
(c) substituting one or more amino acid residues from the identified sequence, wherein the substitutions are made, relative to SEQ ID NO:1, at one or more positions selected from A1, R7, C27, T73, A99, T100, S111, D119, Y120, Y121, H126, L128, Q151, Q165, S168, Q169, I227, S230, N245, M250, N251, A253, S260, V267, Q272, P276, H286, W289, W292, A294, N295, Q297, E301, G311, N325, N327, S333, A334, S336, S339, N341, F353, S359, A360, P363, Q381, Q382, G384, R397, G403, E405, D424, T425, S426, R429, Y432, L436, S437, Q441, Q448, T459, and P464, wherein the position is numbered with reference to the amino acid sequence of SEQ ID NO:1.

In some embodiments, step (c) of the method comprises making one or more amino acid substitutions selected from A1V, R7S, C27Y, T73A, A99P, T100G/N, S111N, D119P/R, Y120H, Y121R, H126E, L128H, Q151L, Q165P/R, S168T, Q169K/L/R, I227A/G/H/K/M/Q, S230P, N245T, M250G, N251D/T, A253P/T, S260K, V267E/K/L, Q272R, P276T, H286Q/S, W289C/M/S, W292A/H/P/R, A294R, N295R, Q297K/P/R/Y, E301K, G311Q, N325H, N327L, S333F, A334P, S336H/K/N/P/T, S339R/Q/W, N341V, F353I, S359D/K, A360C/K/T, P363D/H/V, Q381L, Q382R, G384T, R397H, G403T, E405G/P, D424N/Q, T425K/P/R, S426K, R429D/H/N, Y432W, L436K, S437G/P, Q441K, Q448K, T459G/K/N/R, and P464R.

In some embodiments, the method further comprises determining whether the one or more amino acid substitutions increase the thermostability and/or thermoactivity of the cellobiohydrolase variant in comparison to wild-type M. thermophila CBH2b (SEQ ID NO:1).

In some embodiments, the present invention relates to a method of making M. thermophila CBH2b variants having improved thermoactivity and/or improved thermostability. In some embodiments, the method comprises:
(a) identifying a sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:1;
(b) aligning the identified sequence with the sequence of SEQ ID NO:1; and (c) substituting one or more amino acid residues from the identified sequence, wherein the substitutions are made, relative to SEQ ID NO:1, at one or more positions selected from P2, E6, R7, Q8, A12, W14, G18, N20, G21, A29, T33, A36, Q37, W40, N47, Q49, V50, P56, T61, R64, S67, R74, G76, S81, T83, P86, P87, V92, S94, I95, P96, A99, T100, S101, T102, S106, G107, G112, V113, A117, N118, Y120, S123, H126, L128, I130, S132, M133, A139, S142, A143, E146, Q151, V157, I159, D160, T161, L162, M163, V164, Q165, T166, S168, Q169, A176, A178, N179, P181, S206, N209, G210, A212, A213, K224, I227, E228, S230, M243, V247, T248, N249, V252, A253, S256, A259, S260, V267, K271, Q272, Q297, N308, G311, K312, A332, S336, S339, P340, N341, F353, S354, L356, N358, S359, A360, P363, A364, R365, Q382, G384, V396, A400, N401, H404, E405, A427, A428, L436, S437, E445, Q448, and T459, wherein the position is numbered with reference to SEQ ID NO:1.

In some embodiments, step (c) of the method comprises making one or more amino acid substitutions selected from P2H/S, E6N, R7H/S, Q8L/P, A12I, W14L, G18D, N20L/S, G21D/K, A29R/T, T33H, A36E, Q37F/H/L, W40L, N47K, Q49K, V50D/E/H/K/R, P56T, T61A, R64C, S67G, R74S, G76D, S81P, T83D, P86T, P87T, V92D/K/R/S, S94N, I95H/N, P96E/S, A99V, T100V, S101G, T102C/W, S106W/Y, G107D, G112E, V113I, A117T, N118D, Y120E/N/R, S123RN, H126E/L/M, L128E/H, I130V, S1321, M133F/V, A139H/T, S142E, A143M, E146L, Q151I/L, V157D/H/S, I159S, D160H, T161N/S, L162I, M163A/L, V164E/R, Q165P/T, T166R, S168G/Q/R, Q169D/R, A176G/R, A178N, N179D, P181A, S206H/K, N209S, G210A, A212C/L/N/P/R/S, A213G/H/Q, K224A/E/W, I227A/H/K/M/T, E228G, S230P, M243I, V247A, T248S, N249D/S, V252N, A253N/P/T, S256R, A259E, S260D/K, V267L, K271A, Q272H, Q297R, N308E, G311D, K312A, A332S, S336A/E/L/N/T, S339E/L/Q/V, P340N, N341D, F353L, S354G, L356E/G/H, N358E, S359D/Y, A360D/E/Q/R/S/T/V, P363D, A364T, R365G/L, Q382A/D/H/R, G384S, V396E/R, A400V, N401D, H404N, E405P/Q, A427T, A428N/S, L436D/N, S437P, E445D, Q448T, and T459R.

In some embodiments, the method further comprises determining whether the one or more amino acid substitutions increases the thermoactivity and/or thermostability of the cellobiohydrolase variant in comparison to wild-type M. thermophila CBH2b (SEQ ID NO:1) in an assay, e.g., performed at about 55° C.

### ProSAR Analysis of Cellobiohydrolase Variants

Cellobiohydrolase variants having one or more amino acid substitutions relative to a wild-type cellobiohydrolase, such as M. thermophila CBH2b, can be experimentally generated and characterized for improved properties such as increased thermostability or increased thermoactivity as compared to wild-type cellobiohydrolase. Such experimentally produced variants can subsequently be statistically analyzed in order to determine which amino acid substitution or substitutions are particularly beneficial or detrimental in conferring the desired property (e.g., improved thermostability or improved thermoactivity).

Sequence-activity analysis of variants was performed in accordance with the methods described in U.S. Pat. No. 7,793,428; R. Fox et al., 2003, “Optimizing the search algorithm for protein engineering by directed evolution,” Protein Eng. 16(8):589-597, and R. Fox et al., 2005, “Directed molecular evolution by machine learning and the influence of nonlinear interactions,” J. Theor. Biol. 234(2):187-199, all of which are incorporated herein by reference, to determine whether a mutation has a beneficial, neutral, or deleterious effect on stability or activity when combined with other mutations.

As described herein, substitutions at the following positions were identified as being beneficial for increasing thermostability and/or thermoactivity: R7, R64, A99, T100, S101, S104, D119, Y120, A139, Q165, Q169, I227, S230, A253, Q297, E301, G311, A334, S336, S339, A360, K390, G395, E405, A428, S437, T459, and F465.

Certain cellobiohydrolase variants of the present invention have an amino acid sequence that includes at least one amino acid substitution from one or more amino acid residues selected from R7, A99, T100, Y120, Q169, I227, S230, A253, Q297, E301, A334, S336, S339, A360, S437, and T459, wherein the amino acid residues are numbered with reference to SEQ ID NO:1. Amino acid substitutions at one or more of these positions are predicted to be beneficial substitutions for increasing cellobiohydrolase thermostability and/or thermoactivity. In some embodiments, a cellobiohydrolase variant of the present invention has an amino acid sequence that comprises one or more amino acid substitutions selected from R7S, A99P, T100G, Y120H, Q169R, I227M, S230P, A253P/T, Q297K, E301K, A334P, S336K/N/T, S339W, A360T, S437P, and T459N/R/G, which are predicted to be beneficial substitutions for increasing thermostability and/or thermoactivity.

Certain cellobiohydrolase variants of the present invention have an amino acid sequence that includes an amino acid substitution at one or more positions selected from A99, S230, A253, A334, E405, and S437, wherein the amino acid positions are numbered with reference to SEQ ID NO:1. In some embodiments, the one or more amino acid substitutions are selected from A99P, S230P, A253P/T, A334P, and S437P, which are predicted to be beneficial substitutions for increasing cellobiohydrolase thermostability and/or thermoactivity. In some embodiments, a cellobiohydrolase variant of the present invention has an amino acid sequence having the amino acid substitution A99P. In some embodiments, a cellobiohydrolase variant of the present invention has an amino acid sequence having the amino acid substitution S230P. In some embodiments, a cellobiohydrolase variant of the present invention has an amino acid sequence having the amino acid substitution A253P. In some embodiments, a cellobiohydrolase variant of the present invention has an amino acid sequence having the amino acid substitution A253T. In some embodiments, a cellobiohydrolase variant of the present invention has an amino acid sequence having the amino acid substitution A334P. In some embodiments, a cellobiohydrolase variant of the present invention has an amino acid sequence having the amino acid substitution S437P.

Certain cellobiohydrolase variants of the present invention have an amino acid sequence that includes an amino acid substitution at one or more positions selected from R64, S104, K390, and A428, wherein the amino acid positions are numbered with reference to SEQ ID NO:1. In some embodiments, the one or more amino acid substitutions are selected from R64P, S1041, K390N, and A428T, which are predicted to be beneficial substitutions for increasing cellobiohydrolase thermostability and/or thermoactivity.

Certain cellobiohydrolase variants of the present invention have an amino acid sequence that includes an amino acid substitution at one or more positions selected from R7, T100, Y120, Q169, I227, A253, Q297, E301, S336, S339, A360, and T459, wherein the amino acid positions are numbered with reference to SEQ ID NO:1. In some embodiments, the one or more amino acid substitutions are selected from R7S, T100G, Y120H, Q169R, I227M, A253T, Q297K, E301K, S336K/N/T, S339W, A360T, and T459N/R/G, which are predicted to be beneficial substitutions for increasing cellobiohydrolase thermostability and/or thermoactivity. In some embodiments, a cellobiohydrolase variant of the present invention has an amino acid sequence having the amino acid substitution R7S. In some embodiments, a cellobiohydrolase variant of the present invention has an amino acid sequence having the amino acid substitution T100G. In some embodiments, a cellobiohydrolase variant of the present invention has an amino acid sequence having the amino acid substitution Y120H. In some embodiments, a cellobiohydrolase variant of the present invention has an amino acid sequence having the amino acid substitution Q169R. In some embodiments, a cellobiohydrolase variant of the present invention has an amino acid sequence having the amino acid substitution I227M. In some embodiments, a cellobiohydrolase variant of the present invention has an amino acid sequence having the amino acid substitution A253T. In some embodiments, a cellobiohydrolase variant of the present invention has an amino acid sequence having the amino acid substitution Q297K. In some embodiments, a cellobiohydrolase variant of the present invention has an amino acid sequence having the amino acid substitution E301K. In some embodiments, a cellobiohydrolase variant of the present invention has an amino acid sequence having the amino acid substitution S336K. In some embodiments, a cellobiohydrolase variant of the present invention has an amino acid sequence having the amino acid substitution S336N. In some embodiments, a cellobiohydrolase variant of the present invention has an amino acid sequence having the amino acid substitution S336T. In some embodiments, a cellobiohydrolase variant of the present invention has an amino acid sequence having the amino acid substitution S339W. In some embodiments, a cellobiohydrolase variant of the present invention has an amino acid sequence having the amino acid substitution A360T. In some embodiments, a cellobiohydrolase variant of the present invention has an amino acid sequence having the amino acid substitution T459N. In some embodiments, a cellobiohydrolase variant of the present invention has an amino acid sequence having the amino acid substitution T459R. In some embodiments, a cellobiohydrolase variant of the present invention has an amino acid sequence having the amino acid substitution T459G.

Certain cellobiohydrolase variants of the present invention have an amino acid sequence that includes an amino acid substitution at one or more positions selected from S101, D119, A139, Q165, I227, Q297, G311, S339, L356, S359, A360, A428, S437, and F465, wherein the amino acid positions are numbered with reference to SEQ ID NO:1. In some embodiments, the one or more amino acid substitutions are selected from S101R, D119R, A139P, Q165R, I227Q, Q297R, G311Q, S339Q, L356P, S359D, A360K, A428P, S437G, F465R, which are predicted to be beneficial substitutions for increasing cellobiohydrolase thermostability and/or thermoactivity.

Certain cellobiohydrolases variant of the present invention have an amino acid sequence that includes an amino acid substitution at one or more positions selected from Y120, I227, E301, and T459, wherein the amino acid positions are numbered with reference to SEQ ID NO:1. In some embodiments, the one or more amino acid substitutions are selected from Y120H, I227M, E301K, and T459N/R, which are predicted to be beneficial substitutions for increasing cellobiohydrolase thermostability and/or thermoactivity. In some embodiments, a cellobiohydrolase variant of the present invention has an amino acid sequence having the amino acid substitution Y120H. In some embodiments, a cellobiohydrolase variant of the present invention has an amino acid sequence having the amino acid substitution I227M. In some embodiments, a cellobiohydrolase variant of the present invention has an amino acid sequence having the amino acid substitution E301K. In some embodiments, a cellobiohydrolase variant of the present invention has an amino acid sequence having the amino acid substitution T459N. In some embodiments, a cellobiohydrolase variant of the present invention has an amino acid sequence having the amino acid substitution T459R.

Certain cellobiohydrolase variants of the present invention have an amino acid sequence that includes an amino acid substitution at one or more positions selected from Q165, S339, and G395, wherein the amino acid positions are numbered with reference to SEQ ID NO:1. In some embodiments, the one or more amino acid substitutions are selected from Q165R, S339Q, and G395C, which are predicted to be beneficial substitutions for increasing cellobiohydrolase thermostability and/or thermoactivity.

### III. Exemplary Substitutions in Cellobiohydrolase Homologs

In another aspect, the present invention contemplates that substitutions may be introduced into type 2 cellobiohydrolases of fungal species other than M. thermophila, at positions corresponding to the amino acid positions of wild-type M. thermophila CBH2b (SEQ ID NO:1), to produce variants having increased thermostability and/or thermoactivity. Cellobiohydrolase type 2 belongs to the glycoside hydrolase family 6 (GH6) family of cellulases (formerly known as cellulase family B), a group of enzymes that hydrolyze glycosidic bonds in cellulose. The GH6 cellulase cellobiohydrolase type 2 generally has a cellulose-binding domain (CBD), a catalytic domain that hydrolyzes cellulose, and a linker peptide joining the CBD and catalytic domains.

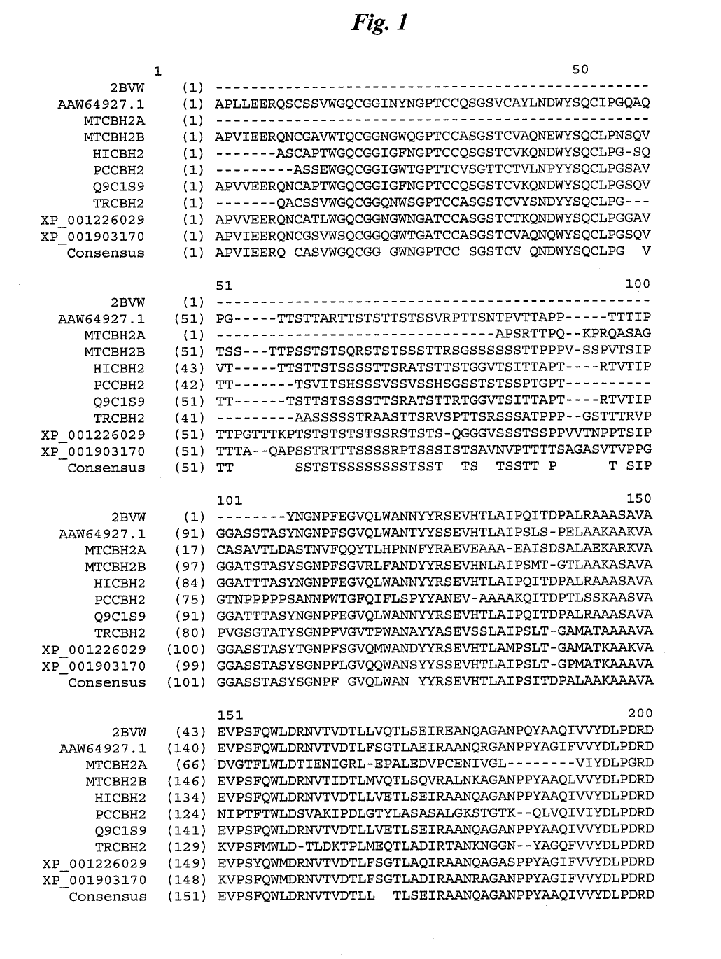

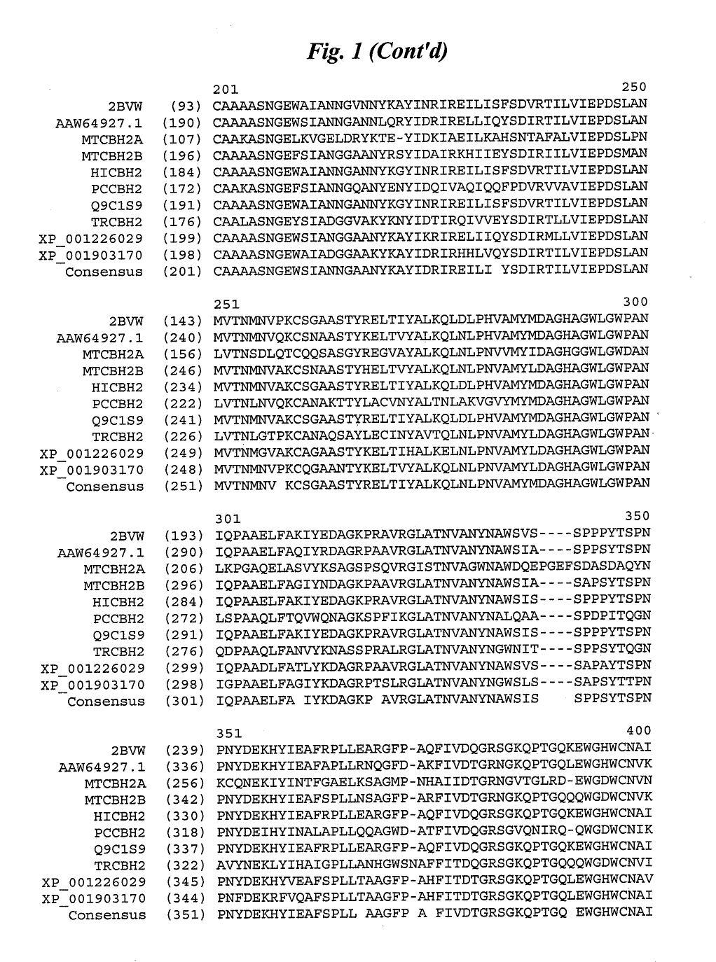

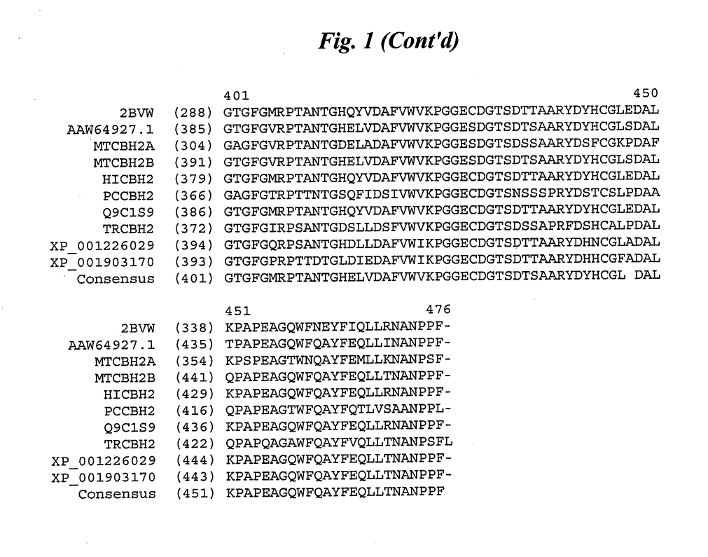

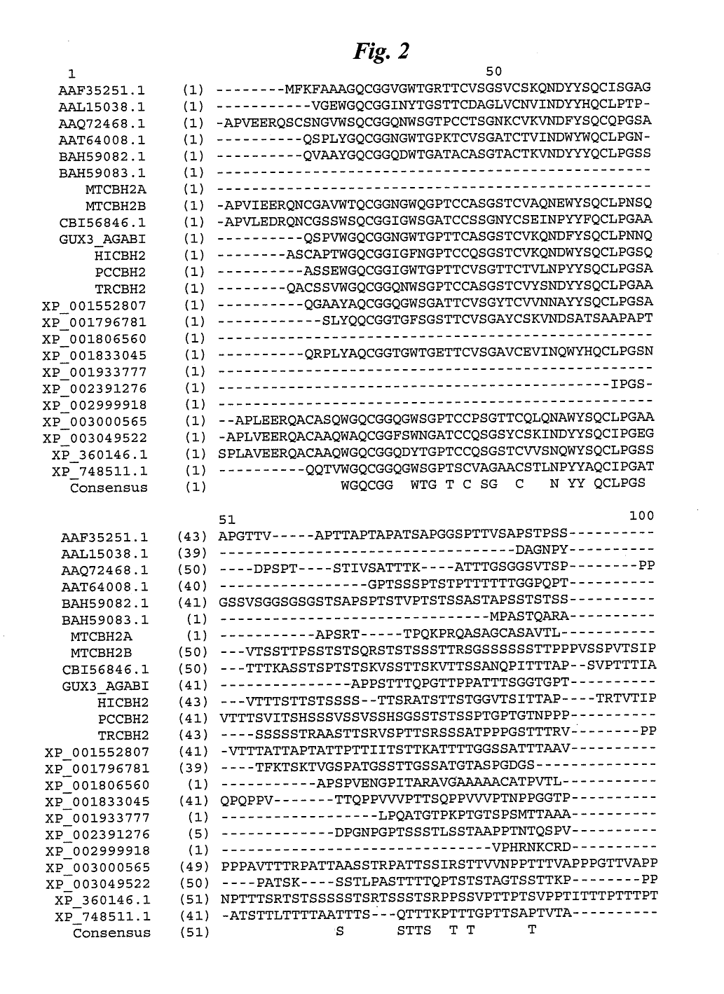

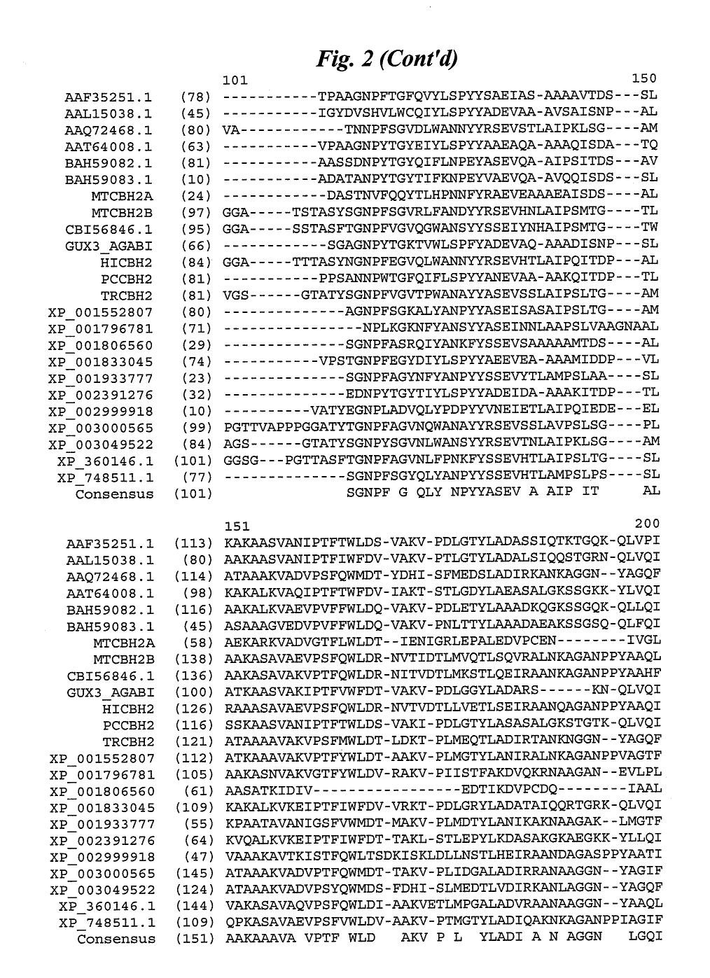

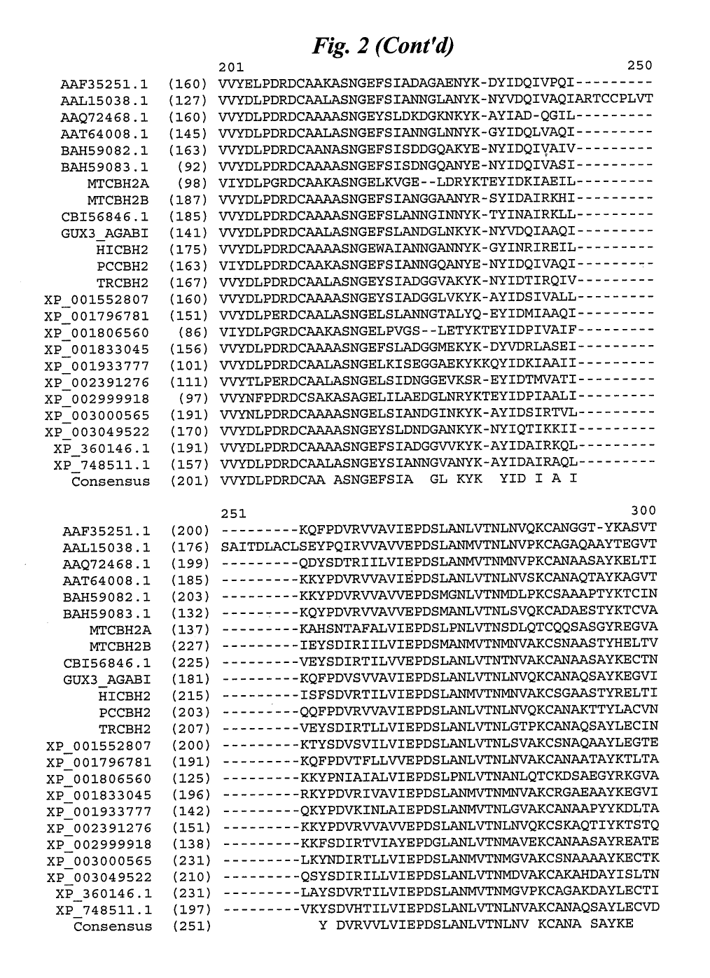

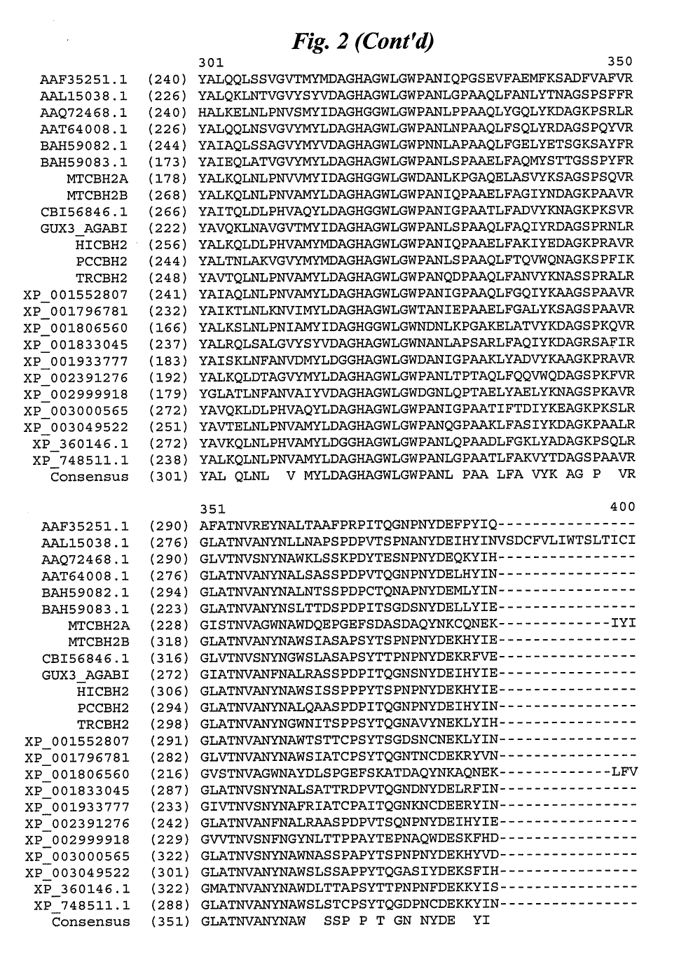

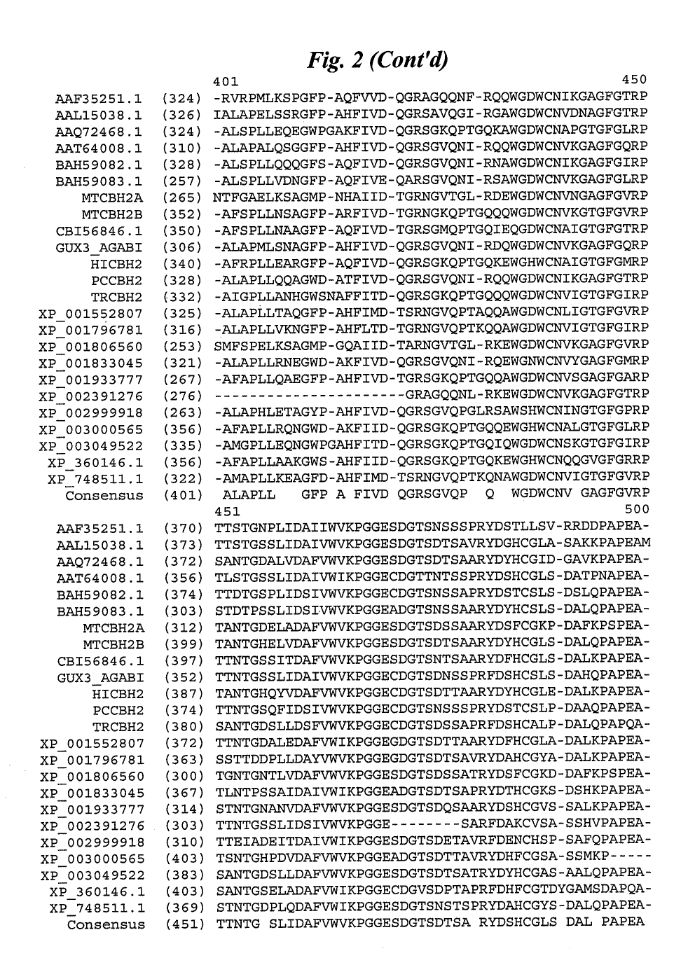

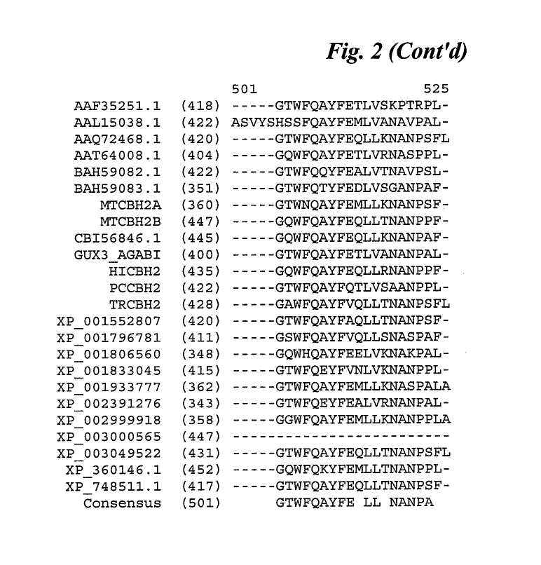

FIGS. 1 and 2 show that there is a high degree of conservation of primary amino acid sequence structure among many cellobiohydrolase type 2 homologs. Alignments across 10 or 25 cellobiohydrolase type 2 homologs of fungal origin shows that these homologs exhibit about 49% sequence homology or greater to M. thermophila CBH2b (SEQ ID NO:1) across the length of the entire mature protein.

For example, a number of fungal strains (including, but not limited to, Acremonium, Agaricus, Aspergillus, Chaetomium, Chrysosporium, Cochliobolus, Coniophora, Coprinopsis, Fusarium, Gibberella, Humicola, Hypocrea, Leptosphaeria, Magnaporthe, Neurospora, Penicillium, Phanerochaete, Podospora, Talaromyces, Thielavia, Trametes, Trichoderma, and Volvariella) express cellobiohydrolase homologs with significant sequence identity to M. thermophila cellobiohydrolase.

In some embodiments, a recombinant cellobiohydrolase of the present invention is derived from a fungal protein shown in Table 1.

TABLE 1
Cellobiohydrolase homologs having significant sequence
identity to M. thermophila CBH2b

| | | | % Homology to |
|---|---|---|---|
| | | SEQ ID | M. thermophila |
| Organism | Protein | NO | CBH2b |
| M. thermophila | Cellobiohydrolase type IIb | 1 | — |
| | (CBH2b) | | |
| Humicola insolens | Cellobiohydrolase type II | 5 | 79% |
| | (Cel6A) | | |
| Chaetomium thermophilum | Cellobiohydrolase | 6 | 77% |
| | (Cel6A) | | |
| Humicola insolens | Cellobiohydrolase type II | 7 | 77% |
| | (Cel6A) | | |
| Chaetomium globosum CBS | Unnamed protein | 8 | 77% |
| 148.51 | | | |
| Humicola insolens | Cellobiohydrolase type II | 9 | 76% |
| | (Cel6A) | | |
| Podospora anserina S mat+ | Unnamed protein | 10 | 74% |
| Sordaria macrospora | Unnamed protein | 11 | 70% |
| Botryotinia fuckeliana B05.10 | Unnamed protein | 12 | 66% |
| Nectria haematococca mpVI 77- | Unnamed protein | 13 | 66% |
| 13-4 | | | |
| Aspergillus fumigatus Af293 | Cellobiohydrolase | 14 | 66% |
| | (CBH) | | |
| Trichoderma reesei | Cellobiohydrolase type II | 15 | 65% |
| | (CBH2) | | |
| Gibberella zeae | Cellulase | 16 | 64% |
| | (Cel6) | | |
| Magnaporthe oryzae 70-15 | Unnamed protein | 17 | 64% |
| Pyrenophora tritici-repentis Pt- | Exoglucanase-6A | 18 | 61% |
| 1C-BFP | | | |
| Verticillium albo-atrum | Exoglucanase | 19 | 60% |
| VaMs.102 | | | |
| Phaetosphaeria nodorum SN15 | Unnamed protein | 20 | 59% |
| Agaricus bisporus | Exoglucanase 3 | 21 | 58% |
| Volvariella volvacea | Cellobiohydrolase type II-I | 22 | 56% |
| | (CBH2-1) | | |
| Coniophora puteana | Cel6B | 23 | 55% |
| Phaenerochaete chrysosporium | Cellobiohydrolase type II | 24 | 54% |
| | (CBH2) | | |
| Lentinus sajor-caju | Cellobiohydrolase type II | 25 | 53% |
| | (CBH2) | | |
| Coniophora puteana | Cellulase | 26 | 53% |
| | (Cel6A) | | |
| Verticillium albo-atrum | | 27 | 52% |
| VaMs.102 | | | |
| Coprinopsis cinerea | Exocellobiohydrolase | 28 | 51% |
| okayama7#130 | | | |
| Moniliophthora perniciosa | Unnamed protein | 29 | 51% |
| FA553 | | | |
| M. thermophila | Cellobiohydrolase type IIa | 30 | 50% |
| | (CBH2a) | | |
| Phaetosphaeria nodorum SN15 | Unnamed protein | 31 | 50% |
| Trametes versicolor | Cellobiohydrolase | 32 | 49% |
| | (Cor1) | | |

It is within the ability of one of ordinary skill in the art to identify other examples of structurally homologous proteins. The present invention provides variants of these and other homologous cellobiohydrolase proteins in which substitutions are made at residues corresponding to those identified herein in the M. thermophila CBH2b protein.

It is possible to use sequence alignment or other methods to identify amino acid positions in structurally related proteins (i.e., homologs) that correspond to each other. Corresponding positions in homologs are considered Performance Sensitive Positions (PSPs) when a substitution in that position is determined to affect a property in a set of multiple homologs. Substitutions at PSPs in other homologs are expected to also have significant effects on activity. For example, mutations in M. thermophila CBH2b which were shown by experimental data to exhibit increased thermostability or increased thermoactivity as compared to wild-type CBH2b (identified in Tables 3, 4, and 6, infra) were mapped to the cellobiohydrolase sequence alignment of FIG. 1. The amino acid sequence of cellobiohydrolase homolog M. thermophila CBH2a was aligned to M. thermophila CBH2b, and beneficial substitutions at residues corresponding to experimentally identified beneficial substitutions in M. thermophila CBH2b were identified. Experimental data was used which was generated from screening variants of wild-type M. thermophila CBH2a for increased thermostability. Experimental data for the M. thermophila CBH2a corresponding residues is shown in Example 10. Residues for which improved CBH2 performance is found for M. thermophila CBH2b as well as M. thermophila CBH2a are identified as “performance sensitive positions,” positions where amino acid substitutions are likely to have a beneficial effect on CBH2 performance.

As shown in Tables 2a and 2b, PSPs were identified for some variants of CBH2a and CBH2b. Table 2a shows PSPs identified based on beneficial thermostability mutations identified in Tables 3 and/or 4. Table 2b shows PSPs identified based on beneficial thermoactivity mutations identified in Table 6. In some embodiments, an amino acid position where a substitution has been shown to be beneficial for increasing cellobiohydrolase thermostability and/or thermoactivity in M. thermophila cellobiohydrolases is also predicted to be a position wherein a substitution from the naturally-occurring amino acid residue at that position will be beneficial for increasing cellobiohydrolase thermostability and/or thermoactivity in a homolog of M. thermophila cellobiohydrolase. In some embodiments, the amino acid position wherein a substitution from the naturally-occurring amino acid residue at that position is beneficial for increasing cellobiohydrolase thermostability and/or thermoactivity is an amino acid position selected from position 92, position 94, position 95, position 96, position 111, position 111, position 119, position 161, position 176, position 213, position 249, position 250, position 289, position 294, position 336, position 358, position 359, position 384, position 427, position 432, and position 448, wherein the amino acid positions are numbered with reference to SEQ ID NO:1. Thus, in some embodiments, a cellobiohydrolase variant comprises an amino acid substitution from the wild-type amino acid residue at one or more (e.g., 1, 2, 3, 4, 5, 6, 7, 8, 9, 10, or more) amino acid positions selected from position 92, position 94, position 95, position 96, position 111, position 111, position 119, position 161, position 176, position 213, position 249, position 250, position 289, position 294, position 336, position 358, position 359, position 384, position 427, position 432, and position 448, wherein the amino acid positions are numbered with reference to SEQ ID NO:1.

As shown in Table 2a below, in some embodiments, the amino acid position wherein a substitution is beneficial for increasing cellobiohydrolase thermostability and/or thermoactivity is an amino acid position selected from position 111, position 119, position 250, position 289, position 294, position 336, position 359, position 384, position 432, and position 448, wherein the amino acid positions are numbered with reference to SEQ ID NO:1. In some embodiments, the amino acid residue at position 111, position 119, position 250, position 289, position 294, position 336, position 359, position 384, position 432, and/or position 448 (numbered with reference to SEQ ID NO:1) that is beneficial for increasing cellobiohydrolase thermostability and/or thermoactivity is any amino acid residue other than the amino acid residue that naturally occurs at that position of the cellobiohydrolase. In some embodiments, an amino acid substitution at position 111 is selected from cysteine and asparagine (X111C/N). In some embodiments, an amino acid substitution at position 119 is selected from alanine, cysteine, proline, valine, and a basic amino acid (X119A/C/P/V/K/R). In some embodiments, an amino acid substitution at position 250 is selected from cysteine, glycine, leucine, and methionine (X250C/G/L/M). In some embodiments, an amino acid substitution at position 289 is selected from cysteine, methionine, serine, and threonine (X289C/M/S/T). In some embodiments, an amino acid substitution at position 294 is selected from arginine and tryptophan (X294R/W). In some embodiments, an amino acid substitution at position 336 is selected from a basic amino acid, asparagine, proline, and threonine (X336H/K/N/P/T). In some embodiments, an amino acid substitution at position 359 is selected from alanine, aspartic acid, and lysine (X359A/D/K). In some embodiments, an amino acid substitution at position 384 is selected from methionine and threonine (X384M/T). In some embodiments, an amino acid substitution at position 432 is selected from proline and tryptophan (X432P/W). In some embodiments, an amino acid substitution at position 448 is selected from glutamic acid, lysine, and glutamine (X448E/K/Q).

TABLE 2a
Performance Sensitive Positions Identified Based
on Variants Identified in Tables 3 and 4

| Performance Sensitive | Beneficial Mutations | Beneficial |
|---|---|---|
| Position (numbered | in M. thermophila | Mutations |
| according to M. thermophila | CBH2b | in M. thermophila |
| CBH2b) | (SEQ ID NO: 1) | CBH2a |
| 111 | S111N | Q31C |
| 119 | D119PR | N39ACKPRV |
| 250 | M250G | S160CLM |
| 289 | W289CMS | W199MT |
| 294 | A294R | A204W |
| 336 | S336HKNPT | S250P |
| 359 | S359DK | S273A |
| 384 | G384T | G297M |
| 432 | Y432W | S345P |
| 448 | Q448K | T361EKQ |

As shown in Table 2b below, in some embodiments, the amino acid position wherein a substitution is beneficial for increasing cellobiohydrolase thermostability and/or thermoactivity is an amino acid position selected from position 92, position 94, position 95, position 96, position 161, position 176, position 213, position 249, position 336, position 358, position 359, position 384, position 427, and position 448, wherein the amino acid positions are numbered with reference to SEQ ID NO:1. In some embodiments, the amino acid residue at position 92, position 94, position 95, position 96, position 161, position 176, position 213, position 249, position 336, position 358, position 359, position 384, position 427, and/or position 448 (numbered with reference to SEQ ID NO:1) that is beneficial for increasing cellobiohydrolase thermostability and/or thermoactivity is any amino acid residue other than the amino acid residue that naturally occurs at that position of the cellobiohydrolase. In some embodiments, an amino acid substitution at position 92 is selected from aspartic acid, isoleucine, lysine, asparagine, arginine, serine, and threonine (X92D/I/K/N/R/S/T). In some embodiments, an amino acid substitution at position 94 is selected from asparagine and proline (X94N/P). In some embodiments, an amino acid substitution at position 95 is selected from histidine, leucine, and asparagine (X95H/L/N). In some embodiments, an amino acid substitution at position 96 is selected from glutamic acid, phenylalanine, isoleucine, and serine (X96E/F/I/S). In some embodiments, an amino acid substitution at position 161 is selected from lysine, asparagine, and serine (X161K/N/S). In some embodiments, an amino acid substitution at position 176 is selected from alanine, leucine, and arginine (X176G/L/R). In some embodiments, an amino acid substitution at position 213 is selected from glycine, histidine, glutamine, and serine (X213G/H/Q/S). In some embodiments, an amino acid substitution at position 249 is selected from aspartic acid, histidine, and serine (X249D/H/S). In some embodiments, an amino acid substitution at position 336 is selected from alanine, cysteine, glutamic acid, leucine, asparagine, proline, threonine, and valine (X336A/C/E/L/N/P/T/V). In some embodiments, an amino acid substitution at position 358 is selected from alanine and glutamic acid (X358A/E). In some embodiments, an amino acid substitution at position 359 is selected from alanine, aspartic acid, and tyrosine (X359A/D/Y). In some embodiments, an amino acid substitution at position 384 is selected from methionine and serine (X384M/S). In some embodiments, an amino acid substitution at position 427 is selected from serine and threonine (X427$/T). In some embodiments, an amino acid substitution at position 448 is selected from glutamic acid, lysine, glutamine, and threonine (X448E/K/Q/T).

TABLE 2b
Performance Sensitive Positions Identified Based
on Variants Identified in Table 6

| Performance Sensitive | Beneficial Mutations | Beneficial |
|---|---|---|
| Position (numbered | in M. thermophila | Mutations |
| according to M. thermophila | CBH2b | in M. thermophila |
| CBH2b) | (SEQ ID NO: 1) | CBH2a |
| 92 | V92DKRS | R11INTV |
| 94 | S94N | A13P |
| 95 | I95HN | S14L |
| 96 | P96ES | A15FIS |
| 161 | T161NS | R81K |
| 176 | A176GR | V95L |
| 213 | A213GHQ | D122S |
| 249 | N249DS | N159H |
| 336 | S336AELNT | S250CPV |
| 358 | N358E | K272A |
| 359 | S359DY | S273AD |
| 384 | G384S | G297M |
| 427 | A427T | A340S |
| 448 | Q448T | T361EKQ |

Thus, in some embodiments, the present invention provides a recombinant cellobiohydrolase variant comprising at least about 50% sequence identity to wild-type M. thermophila CBH2b (SEQ ID NO:1) and comprising one or more amino acid substitutions, relative to SEQ ID NO:1, selected from:
an aspartic acid, isoleucine, lysine, asparagine, arginine, serine, or threonine residue at position 92 (X92D/I/K/N/R/S/T);
an asparagine or proline residue at position 94 (X94N/P);
a histidine, leucine, or asparagine residue at position 95 (X95H/L/N);
a glutamic acid, phenylalanine, isoleucine, or serine residue at position 96 (X96E/F/I/S);
a cysteine or asparagine residue at position 111 (X111C/N);
an alanine, cysteine, lysine, proline, arginine, or valine residue at position 119 (X119A/C/K/P/R/V);
a lysine, asparagine, or serine residue at position 161 (X161K/N/S);
an alanine, leucine, or arginine residue at position 176 (X176G/L/R);
a glycine, histidine, glutamine, or serine residue at position 213 (X213G/H/Q/S);
an aspartic acid, histidine, or serine residue at position 249 (X249D/H/S);
a cysteine, glycine, leucine, or methionine residue at position 250 (X250C/G/L/M);
a cysteine, methionine, serine, or threonine residue at position 289 (X289C/M/S/T);
a glutamine, arginine, or tryptophan residue at position 294 (X294Q/R/W);
an alanine, cysteine, glutamic acid, histidine, lysine, leucine, asparagine, proline, threonine, or valine residue at position 336 (X336A/C/E/H/K/L/N/P/T/V);
an alanine or glutamic acid residue at position 358 (X358A/E);
an alanine, aspartic acid, lysine, or tyrosine residue at position 359 (X359A/D/K/Y);
a methionine, serine, or threonine residue at position 384 (X384M/S/T); a serine or threonine residue at position 427 (X427S/T);
a glutamic acid, proline, or tryptophan residue at position 432 (X432E/P/W); and
a glutamic acid, lysine, glutamine, or threonine residue at position 448 (X448E/K/Q/T), wherein the position is numbered with reference to SEQ ID NO:1, and wherein the cellobiohydrolase variant has increased thermostability and/or thermoactivity in comparison to the wild-type cellobiohydrolase from which the variant is derived. In some embodiments, the cellobiohydrolase variant is derived from wild-type Myceliophthora thermophila CBH2b (SEQ ID NO:1). In some embodiments, the cellobiohydrolase variant is derived from a homolog of Myceliophthora thermophila CBH2b, e.g., a homolog listed in Table 1 above.

In some embodiments, the present invention provides a recombinant cellobiohydrolase variant comprising at least about 50% sequence identity to wild-type M. thermophila CBH2b (SEQ ID NO:1) and comprising one or more amino acid substitutions, relative to SEQ ID NO:1, selected from:
a cysteine or asparagine residue at position 111 (X111C/N);
an alanine, cysteine, lysine, proline, arginine, or valine residue at position 119 (X119A/C/K/P/R/V);
a cysteine, glycine, leucine, or methionine residue at position 250 (X250C/G/L/M);
a cysteine, methionine, serine, or threonine residue at position 289 (X289C/M/S/T);
an arginine or tryptophan residue at position 294 (X294R/W);
a histidine, lysine, asparagine, proline, or threonine residue at position 336 (X336H/K/N/P/T);
an alanine, aspartic acid, or lysine residue at position 359 (X359A/D/K);
a methionine or threonine residue at position 384 (X384M/T);
a proline or tryptophan residue at position 432 (X432P/W); and
a glutamic acid, lysine, or glutamine residue at position 448 (X448E/K/Q),
   wherein the position is numbered with reference to the amino acid sequence of SEQ ID NO:1, and wherein the variant has increased thermostability and/or thermoactivity in comparison to the wild-type cellobiohydrolase from which the variant is derived. In some embodiments, the cellobiohydrolase variant is derived from wild-type Myceliophthora thermophila CBH2b (SEQ ID NO:1). In some embodiments, the cellobiohydrolase variant is derived from a homolog of Myceliophthora thermophila CBH2b, e.g., a homolog listed in Table 1 above.

In some embodiments, the present invention provides a recombinant cellobiohydrolase variant comprising at least about 50% sequence identity to wild-type M. thermophila CBH2b (SEQ ID NO:1) and comprising one or more amino acid substitutions, relative to SEQ ID NO:1, selected from:
an aspartic acid, isoleucine, lysine, asparagine, arginine, serine, or threonine residue at position 92 (X92D/I/K/N/R/S/T);
an asparagine or proline residue at position 94 (X94N/P);
a histidine, leucine, or asparagine residue at position 95 (X95H/L/N);
a glutamic acid, phenylalanine, isoleucine, or serine residue at position 96 (X96E/F/I/S);
a lysine, asparagine, or serine residue at position 161 (X161K/N/S);
an alanine, leucine, or arginine residue at position 176 (X176G/L/R);
a glycine, histidine, glutamine, or serine residue at position 213 (X213G/H/Q/S);
an aspartic acid, histidine, or serine residue at position 249 (X249D/H/S);
an alanine, cysteine, glutamic acid, leucine, asparagine, proline, threonine, or valine residue at position 336 (X336A/C/E/L/N/P/T/N);
an alanine or glutamic acid residue at position 358 (X358A/E);
an alanine, aspartic acid, or tyrosine residue at position 359 (X359A/D/Y);
a methionine or serine residue at position 384 (X384M/S);
a serine or threonine residue at position 427 (X427S/T); and
a glutamic acid, lysine, glutamine, or threonine residue at position 448 (X448E/K/Q/T), wherein the position is numbered with reference to SEQ ID NO:1, and wherein the cellobiohydrolase variant has increased thermostability and/or thermoactivity in comparison to the wild-type cellobiohydrolase from which the variant is derived. In some embodiments, the cellobiohydrolase variant is derived from wild-type Myceliophthora thermophila CBH2b (SEQ ID NO:1). In some embodiments, the cellobiohydrolase variant is derived from a homolog of Myceliophthora thermophila CBH2b, e.g., a homolog listed in Table 1 above.

In some embodiments, the cellobiohydrolase variant comprises an amino acid substitution at one or more positions selected from position 336, position 359, position 384, and position 448, wherein the position is numbered with reference to SEQ ID NO:1. As described below in Example 13, these amino acid positions were identified as Performance Sensitive Positions based on both beneficial thermostability mutations identified in Tables 3 and/or 4 and beneficial thermoactivity mutations identified in Table 6. Therefore, a substitution from the naturally-occurring amino acid residue at one or more of these positions is expected to be beneficial. In some embodiments, the amino acid residue at position 336, position 359, position 384, and/or position 448 may be any amino acid residue other than the amino acid residue that naturally occurs in the cellobiohydrolase from which the cellobiohydrolase variant is derived (e.g., the wild-type Myceliophthora thermophila CBH2b of SEQ ID NO:1). In some embodiments, the cellobiohydrolase variant comprises one or more amino acid substitutions, relative to SEQ ID NO:1, selected from:
an alanine, cysteine, glutamic acid, histidine, lysine, leucine, asparagine, proline, threonine, or valine residue at position 336 (X336A/C/E/H/K/L/N/P/T/V);
an alanine, aspartic acid, lysine, or tyrosine residue at position 359 (X359A/D/K/Y);
a methionine, serine, or threonine residue at position 384 (X384M/S/T); and
a glutamic acid, lysine, glutamine, or threonine residue at position 448 (X448E/K/Q/T), wherein the position is numbered with reference to SEQ ID NO:1, and wherein the cellobiohydrolase variant has increased thermostability and/or thermoactivity in comparison to the wild-type cellobiohydrolase from which the variant is derived. In some embodiments, the cellobiohydrolase variant is derived from wild-type Myceliophthora thermophila CBH2b (SEQ ID NO:1). In some embodiments, the cellobiohydrolase variant is derived from a homolog of Myceliophthora thermophila CBH2b, e.g., a homolog listed in Table 1 above.

In some embodiments, the cellobiohydrolase variant comprising one or more of said mutations comprises at least about 55%, at least about 60%, about at least 65%, at least about 70% amino acid sequence identity or higher to wild-type M. thermophila CBH2b (SEQ ID NO:1).

In some embodiments, the cellobiohydrolase variant of the present invention is derived from a protein from a fungal strain. In some embodiments, the isolated cellobiohydrolase variant comprises at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a cellobiohydrolase type 2 from M. thermophila (SEQ ID NOs:1 or 30), Humicola insolens (SEQ ID NOs:5, 7, or 9), Chaetomium thermophilum (SEQ ID NO:6), Chaetomium globosum (SEQ ID NO:8), Podospora anserina (SEQ ID NO:10), Sordaria macrospora (SEQ ID NO:11), Botryotinia fuckeliana (SEQ ID NO:12), Nectria haematococca (SEQ ID NO:13), Aspergillus fumigatus (SEQ ID NO:14), Trichoderma reesei (SEQ ID NO:15), Gibberella zeae (SEQ ID NO:16), Magnaporthe oryzae (SEQ ID NO:17), Pyrenophora tritici-repentis (SEQ ID NO:18), Verticillium albo-atrum (SEQ ID NOs:19 or 27), Phaetosphaeria nodorum (SEQ ID NOs:20 or 31), Agaricus bisporus (SEQ ID NO:21), Volvariella volvacea (SEQ ID NO:22), Coniophora puteana (SEQ ID NOs:23 or 26), Phaenerochaete chrysosporium (SEQ ID NO:24), Lentinus sajor-caju (SEQ ID NO:25), Coprinopsis cinerea (SEQ ID NO:28), Moniliophthora perniciosa (SEQ ID NO:29), or Trametes versicolor (SEQ ID NO:32). In some embodiments, a cellobiohydrolase variant of the present invention comprises at least 50%, at least 55%, at least 60%, at least 65%, at least 70%, at least 75%, at least 80%, at least 85%, at least 90%, at least 91%, at least 92%, at least 93%, at least 94%; at least 95%, at least 96%, at least 97%, at least 98%, or at least 99% sequence identity to a cellobiohydrolase sequence described herein and comprises an amino acid substitution at one or more positions homologous to the positions identified in Table 2a (i.e., position 111, position 119, position 250, position 289, position 294, position 336, position 359, position 384, position 432, or position 448 as numbered with reference to SEQ ID NO:1) and/or comprises an amino acid substitution at one or more positions homologous to the positions identified in Table 2b (i.e., position 92, position 94, position 95, position 96, position 161, position 176, position 213, position 249, position 336, position 358, position 359, position 384, position 427, or position 448 as numbered with reference to SEQ ID NO:1).

In some embodiments, a cellobiohydrolase variant having improved thermostability and/or thermoactivity comprises a substitution at residue 336 (numbered with respect to SEQ ID NO:1). Amino acid residue 336 is identified as a Performance Sensitive Position in a CBH2 homolog and is also identified by ProSAR analysis to be a beneficial residue for amino acid substitution in M. thermophila CBH2b. Therefore, a substitution from the naturally-occurring amino acid residue at this position is expected to be beneficial. Thus, in some embodiments, a cellobiohydrolase variant of the present invention has an amino acid sequence comprising an amino acid residue at position 336 (numbered with reference to SEQ ID NO:1) other than the amino acid that naturally occurs at that position in the cellobiohydrolase from which the variant is derived. In some embodiments, a cellobiohydrolase variant of the present invention has an amino acid sequence comprising an alanine, cysteine, glutamic acid, basic, leucine, asparagine, proline, serine, threonine, or valine residue at position 336 (X336A/C/G/H/K/L/N/P/S/T/V), wherein the amino acid positions are numbered with reference to SEQ ID NO:1. This substitution is expected to be beneficial for improving cellobiohydrolase thermostability and/or thermoactivity.

In some embodiments, a cellobiohydrolase variant having improved thermostability and/or thermoactivity comprises a substitution at residue 99 (numbered with respect to SEQ ID NO:1). Amino acid residue 99 is identified as a Performance Sensitive Position in a CBH2 homolog and is also identified by ProSAR analysis to be a beneficial residue for amino acid substitution in M. thermophila CBH2b. Therefore, a substitution at this position is expected to be beneficial. Thus, in some embodiments, a cellobiohydrolase variant of the present invention has an amino acid sequence comprising an amino acid residue at position 99 (numbered with reference to SEQ ID NO:1) other than the amino acid that naturally occurs at that position in the cellobiohydrolase from which the variant is derived. In some embodiments, a cellobiohydrolase variant of the present invention has an amino acid sequence comprising an aspartic acid, glutamic acid, or proline residue at position 99 (X99D/E/P), wherein the amino acid positions are numbered with reference to SEQ ID NO:1. This substitution is expected to be beneficial for improving cellobiohydrolase thermostability and/or thermoactivity.

It will be appreciated that secreted cellobiohydrolase variants of the present invention may encompass additional amino acid substitutions beyond those listed above (such as additional conservative substitutions) or may be less-than-full length compared to a wild-type secreted M. thermophila cellobiohydrolase protein. Thus, cellobiohydrolase variants of the present invention may comprise insertions or may be truncated (at the N- or C-terminus) or comprise deletions relative to a full-length cellobiohydrolase (e.g., SEQ ID NO:1). For illustration and not limitation, in some embodiments the variant may be longer or shorter by up to 25%, 20%, 15%, 10%, 5%, 4%, 3%, 2%, or 1% of the wild-type length. For example, a cellobiohydrolase variant with an amino-terminal and/or carboxy-terminal deletion and/or internal deletion relative to a full-length cellobiohydrolase (e.g., SEQ ID NO:1) may comprise, for example, about 70%, about 75%, about 80%, at about 85%, about 90%, about 91%, about 92%, about 93%, about 94%, about 95%, about 96%, about 97%, about 98%, or about 99% the length of a full-length cellobiohydrolase polypeptide.

In some embodiments, a cellobiohydrolase variant of the present invention further comprises a signal peptide linked to the amino-terminus of the polypeptide. In some embodiments, the signal peptide is an endogenous M. thermophila cellobiohydrolase signal peptide. In some embodiments, the signal peptide is a signal peptide from another M. thermophila secreted protein. In some embodiments, the signal peptide is a signal peptide from a cellobiohydrolase or another secreted protein secreted from an organism other than M. thermophila (e.g., from a filamentous fungus, yeast, or bacteria).

### IV. Generation of Cellobiohydrolase Variants

A cellobiohydrolase variant polypeptide of the invention can be subject to further modification to generate new polypeptides that retain the specific substitutions that characterize the variant and which may have desirable properties. For example, a polynucleotide encoding a cellobiohydrolase with an improved property can be subjected to additional rounds of mutagenesis treatments to generate polypeptides with further improvements in the desired enzyme or enzyme properties.

Given the wild-type M. thermophila CBH2b sequence or the sequence of a wild-type fungal homolog of M. thermophila CBH2b, cellobiohydrolase variants can be generated according to the methods described herein and can be screened for the presence of improved properties, such as increased thermostability or increased thermoactivity. Libraries of cellobiohydrolase variant polypeptides (and/or polynucleotides encoding the variant) may be generated from a parental sequence (e.g., wild-type M. thermophila CBH2b, or a wild-type cellobiohydrolase from another fungal strain such as a cellobiohydrolase of Table 1, or one of the cellobiohydrolase variants exemplified herein), and screened using a high throughput screen to determine improved properties such as increased activity or stability at desired conditions, as described herein. Mutagenesis and directed evolution methods are well known in the art and can be readily applied to polynucleotides encoding cellobiohydrolase variants exemplified herein to generate variant libraries that can be expressed, screened, and assayed using the methods described herein. See, e.g., Ling, et al., 1999, “Approaches to DNA mutagenesis: an overview,” Anal. Biochem., 254(2):157-78; Dale, et al., 1996, “Oligonucleotide-directed random mutagenesis using the phosphorothioate method,” Methods Mol. Biol., 57:369-74; Smith, 1985, “In vitro mutagenesis,” Ann. Rev. Genet., 19:423-462; Botstein, et al., 1985, “Strategies and applications of in vitro mutagenesis,” Science, 229:1193-1201; Carter, 1986, “Site-directed mutagenesis,” Biochem. J., 237:1-7; Kramer, et al., 1984, “Point Mismatch Repair,” Cell, 38:879-887; Wells, et al., 1985, “Cassette mutagenesis: an efficient method for generation of multiple mutations at defined sites,” Gene, 34:315-323; Minshull, et al., 1999, “Protein evolution by molecular breeding,” Current Opinion in Chemical Biology, 3:284-290; Christians, et al., 1999, “Directed evolution of thymidine kinase for AZT phosphorylation using DNA family shuffling,” Nature Biotechnology, 17:259-264; Crameri, et al., 1998, “DNA shuffling of a family of genes from diverse species accelerates directed evolution,” Nature, 391:288-291; Crameri, et al., 1997, “Molecular evolution of an arsenate detoxification pathway by DNA shuffling,” Nature Biotechnology, 15:436-438; Zhang, et al., 1997 “Directed evolution of an effective fucosidase from a galactosidase by DNA shuffling and screening,” Proceedings of the National Academy of Sciences, U.S.A., 94:45-4-4509; Crameri, et al., 1996, “Improved green fluorescent protein by molecular evolution using DNA shuffling,” Nature Biotechnology, 14:315-319; Stemmer, 1994, “Rapid evolution of a protein in vitro by DNA shuffling,” Nature, 370:389-391; Stemmer, 1994, “DNA shuffling by random fragmentation and reassembly: In vitro recombination for molecular evolution,” Proceedings of the National Academy of Sciences, U.S.A., 91:10747-10751; WO 95/22625; WO 97/0078; WO 97/35966; WO 98/27230; WO 00/42651; and WO 01/75767, all of which are incorporated herein by reference.

Cellobiohydrolase variants having the amino acid substitutions described herein can also be synthetically generated. Chemically synthesized polypeptides may be generated using the well-known techniques of solid phase, liquid phase, or peptide condensation techniques, and can include any combination of amino acids as desired to produce the variants described herein. Synthetic amino acids can be obtained from Sigma, Cambridge Research Biochemical, or any other chemical company familiar to those skilled in the art.

In generating variants that comprise substitutions, insertions or deletions at positions in addition to those described supra, the ordinarily skilled practitioner will be aware that certain regions of the cellobiohydrolase protein are less tolerant than others to substitutions (especially non-conservative substitutions). Thus, in some embodiments, variant cellobiohydrolase polypeptides retain conserved residues and functional domains from the parent.

### V. Cellobiohydrolase Thermoactivity and Thermostability

Cellobiohydrolase activity and thermostability can be determined by methods described in the Examples section (e.g., Examples 4, 6, and 12), and/or using any other methods known in the art. Cellobiohydrolase activity may be determined, for example, using an assay that measures the conversion of crystalline cellulose to glucose (e.g., Examples 4 and 6). In some embodiments, cellobiohydrolase activity and thermostability can be determined using methods that measure the conversion of a lignocellulose biomass to glucose (e.g., Example 12). For example, activity may determined using an assay that measures conversion of the biomass to cellobiose using a β-glucosidase to produce glucose in a reaction comprising a biomass substrate such as wheat straw.

For example, cellobiohydrolase activity can be determined using a cellulose assay, in which the ability of the cellobiohydrolase variants to hydrolyze a cellulose substrate, e.g., crystalline cellulose, to cellobiose under specific temperature and/or pH conditions is measured using a β-glucosidase to convert the cellobiose to glucose. Conversion of crystalline cellulose to fermentable sugar oligomers (e.g., glucose) can be determined by art-known means, including but not limited to coupled enzymatic assay and colorimetric assay. For example, glucose concentrations can be determined using a coupled enzymatic assay based on glucose oxidase and horseradish peroxidase (e.g., GOPOD assay) as exemplified in Trinder, P. (1969) Ann. Clin. Biochem. 6:24-27, which is incorporated herein by reference in its entirety. GOPOD assay kits are known in the art and are readily commercially available, e.g., from Megazyme (Wicklow, Ireland). Methods for performing GOPOD assays are known in the art; see, e.g., McCleary et al., J. AOAC Int. 85(5):1103-11 (2002), the contents of which are incorporated by reference herein.

In one exemplary assay, biotransformation reactions are performed by mixing 60 μl clear supernatant with 40 μl of a slurry of crystalline cellulose in 340 mM sodium acetate buffer pH 4.2-5.0 (final concentration: 200 g/L crystalline cellulose; a glass bead/well). Additionally, 50 μl of beta-glucosidase supernatant is added to the reaction mixture for the conversion of cellobiose to glucose. Biotransformation is performed at pH 4.5, 65-70° C. for an appropriate amount of time. Glucose generation is measured using a GOPOD assay. For the GOPOD assay, fermentable sugar oligomer (e.g., glucose) production is measured by mixing 10 μl of the above reaction with 190 μl of GOPOD assay mix. The reactions are allowed to shake for 30 min at room temperature. Absorbance of the solution is measured at 510 nm to determine the amount of glucose produced in the original biotransformation reaction. The amount of glucose produced is measured at 510 nm to calculate cellobiohydrolase activity. Additional methods of cellobiose quantification include chromatographic methods, for example by HPLC as exemplified in the incorporated materials of U.S. Pat. Nos. 6,090,595 and 7,419,809.

In another illustrative assay for determining cellobiohydrolase activity, the ability of the cellobiohydrolase variants to hydrolyze a cellulose substrate to cellobiose (e.g., wheat straw pretreated under acidic conditions), under specific temperature and/or pH conditions is measured using a β-glucosidase to convert the cellobiose to glucose. For example, a supernatant containing the secreted cellobiohydrolase is mixed with a cellulose substrate, e.g., wheat straw pretreated under acidic conditions, in a buffered solution and β-glucosidase is added to convert the cellobiose to glucose. A biotransformation reaction is performed under specific time, temperature, and/or pH conditions, for example, pH 5.0, 55° C., for 24-72 hours. An aliquot of the biotransformation reaction is then assayed for conversion of cellulose (e.g., wheat straw pretreated under acidic conditions) to fermentable soluble sugars (e.g., glucose). Conversion of cellulose substrate (e.g., wheat straw pretreated under acidic conditions) to fermentable sugar oligomers (e.g., glucose) can be determined by art-known means, including but not limited to coupled enzymatic assay and colorimetric assay. For example, glucose concentrations can be determined using a coupled enzymatic assay based on glucose oxidase and horseradish peroxidase (e.g., GOPOD assay) as exemplified in Trinder, P. (1969) Ann. Clin. Biochem. 6:24-27, which is incorporated herein by reference in its entirety. GOPOD assay kits are known in the art and are readily commercially available, e.g., from Megazyme (Wicklow, Ireland). Methods for performing GOPOD assays are known in the art; see, e.g., McCleary et al., J. AOAC Int. 85(5):1103-11 (2002), the contents of which are incorporated by reference herein. Additional methods of cellobiose quantification include chromatographic methods, for example by HPLC as exemplified in the incorporated materials of U.S. Pat. Nos. 6,090,595 and 7,419,809.

In a further illustrative assay, cellobiose hydrolase reactions are performed in a buffered reaction by mixing 100 μl of supernatant obtained from a high throughput growth procedure (e.g., a procedure of Example 4) with 200 μl of a pre-treatment filtrate obtained from acid-pretreated wheat straw substrate. In typical embodiments, the filtrate is pH-adjusted to reduce the acidity, e.g., pH-adjusted to a pH of from about 4.0 to about 7.0 Additionally, 100 μl of beta-glucosidase supernatant (to produce 0.005 g/L final β-glucosidase concentration) is added to the reaction mixture for the conversion of cellobiose to glucose. Reactions are incubated at pH 5.0, 55° C. for an appropriate length of time, e.g., 72 hours. Production of glucose can be measured as described above and in Example 12.

Cellobiohydrolase thermostability can be determined, for example, by exposing the cellobiohydrolase variants and the reference (e.g., wild-type) cellobiohydrolase to stress conditions of elevated temperature and/or low pH for a desired period of time and then determining residual cellobiohydrolase activity using an assay that measures the conversion of cellulose to glucose. In an exemplary assay, thermostability is screened using a cellulose-based High Throughput Assay. In deep, 96-well microtiter plates 85 μL of media supernatant containing cellobiohydrolase variant is added to 200 g/L crystalline cellulose in 150 mM sodium acetate buffer pH 4.5. After sealing with aluminum/polypropylene laminate heat seal tape (Velocity 11 (Menlo Park, Calif.), Cat#06643-001)), the plates are shaken at 67° C. for 1 hr. The reactions are diluted by adding 150 μL of water into the deep well plates. The plates are centrifuged at 4000 rpm for 5 minutes. 150 μL of supernatant from the reaction mixture is filtered with a 0.45 μm low-binding hydrophilic PTFE filter plate (Millipore, Billerica, Mass.). The sample plates are sealed with heat seal tape to prevent evaporation. Beta-glucosidase, which converts cellobiose to glucose, is subsequently added and conversion of crystalline cellulose to fermentable sugar is measured by any art-known means, for example using any of the assays as described above, such as coupled enzymatic assay based on glucose oxidase and horseradish peroxidase or GOPOD assay.

Some cellobiohydrolase variants of the present invention will have improved thermoactivity or thermostability as compared to a reference sequence. In some embodiments, a cellobiohydrolase variant has improved thermostability or improved thermoactivity at a pH range of 3.0 to 7.5, at a pH range of 3.5 to 6.5, at a pH range of 3.5 to 6.0, at a pH range of 3.5 to 5.5, at a pH range of 3.5 to 5.0, or at a pH range of 4.0 to 5.0. In some embodiments, a cellobiohydrolase variant has improved thermostability or improved thermoactivity at a temperature of about 55° C. to 80° C., at a temperature of about 60° C. to 80° C., at a temperature of about 65° C. to 80° C., or at a temperature of about 65 to 75° C. In some embodiments, a cellobiohydrolase will have improved thermostability or improved thermoactivity at a pH of 3.5 to 5.0 and a temperature of 65-80° C.

In some embodiments, the cellobiohydrolase variants of the invention exhibit cellobiohydrolase activity that is at least about 1.1 fold, at least about 1.5 fold, at least about 2.0 fold, or at least about 3.0 fold or greater than the cellobiohydrolase activity of a control cellobiohydrolase (e.g., the wild-type cellobiohydrolase of SEQ ID NO:1) when tested under the same conditions. In some embodiments, the thermostability of the cellobiohydrolase variants at pH 4.5 and 70° C. is at least about 1.1 fold, at least about 1.5 fold, at least about 2.0 fold, or at least about 3.0 fold or greater than the thermostability of a control cellobiohydrolase (e.g., the wild-type cellobiohydrolase of SEQ ID NO:1) under the same conditions.

In some embodiments, a cellobiohydrolase variant of the invention will exhibit improved cellobiohydrolase activity as compared to a reference sequence when assayed using a biomass substrate at pH 5.0 at about 55° C. for 72 hours.

### VI. Fusion Peptides and Additional Sequence Elements

In some embodiments, a cellobiohydrolase variant of the present invention further comprises additional sequences which do not alter the encoded activity of the cellobiohydrolase. For example, the cellobiohydrolase may be linked to an epitope tag or to another sequence useful in purification.

The present invention also provides cellobiohydrolase variant fusion polypeptides, wherein the fusion polypeptide comprises an amino acid sequence encoding a cellobiohydrolase variant polypeptide of the present invention or fragment thereof, linked either directly or indirectly through the N- or C-terminus of the cellobiohydrolase variant polypeptide to an amino acid sequence encoding at least a second (additional) polypeptide. The cellobiohydrolase variant fusion polypeptide may further include amino acid sequence encoding a third, fourth, fifth, or additional polypeptides. Typically, each additional polypeptide has a biological activity, or alternatively, is a portion of a polypeptide that has a biological activity, where the portion has the effect of improving expression and/or secretion and/or purification and/or detection of the fusion polypeptide from the desired expression host. These sequences may be fused, either directly or indirectly, to the N- or C-terminus of the cellobiohydrolase variant polypeptide or fragment thereof, or alternatively, to the N- or C-terminus of the additional polypeptides having biological activity.

In some embodiments, the additional polypeptide(s) encode an enzyme or active fragment thereof, and/or a polypeptide that improves expression and/or secretion of the fusion polypeptide from the desired expression host cell. For example, the additional polypeptide may encode a cellulase (for example, a cellobiohydrolase having a different amino acid sequence from the cellobiohydrolase variant polypeptide in the fusion polypeptide, or a polypeptide exhibiting endoglucanase activity or β-glucosidase activity) and/or a polypeptide that improves expression and secretion from the desired host cell, such as, for example, a polypeptide that is normally expressed and secreted from the desired expression host, such as a secreted polypeptide normally expressed from filamentous fungi. These include glucoamylase, α-amylase and aspartyl proteases from Aspergillus niger, Aspergillus niger var. awamori, and Aspergillus oryzae, cellobiohydrolase I, cellobiohydrolase II, endoglucanase I and endoglucase III from Trichoderma and glucoamylase from Neurospora and Humicola species. See WO 98/31821, which is incorporated herein by reference.

The polypeptide components of the fusion polypeptide may be linked to each other indirectly via a linker. Linkers suitable for use in the practice of the present invention are described in WO 2007/075899, which is incorporated herein by reference. Exemplary linkers include peptide linkers of from 1 to about 40 amino acid residues in length, including those from about 1 to about 20 amino acid residues in length, and those from about 1 to about 10 amino acid residues in length. In some embodiments, the linkers may be made up of a single amino acid residue, such as, for example, a Gly, Ser, Ala, or Thr residue or combinations thereof, particularly Gly and Ser. Linkers employed in the practice of the present invention may be cleavable. Suitable cleavable linkers may contain a cleavage site, such as a protease recognition site. Exemplary protease recognition sites are well known in the art and include, for example, Lys-Arg (the KEX2 protease recognition site, which can be cleaved by a native Aspergillus KEX2-like protease), Lys and Arg (the trypsin protease recognition sites). See, for example, WO 2007/075899.

### Signal Peptides

In some embodiments, the cellobiohydrolase variant polypeptides of the present invention are secreted from the host cell in which they are expressed (e.g., a yeast or fungal cell) and are expressed as a pre-protein including a signal peptide, i.e., an amino acid sequence linked to the amino terminus of a polypeptide and which directs the encoded polypeptide into the cell secretory pathway. In one embodiment, the signal peptide is an endogenous M. thermophila cellobiohydrolase signal peptide. For example, the signal peptide of the CBH2b of SEQ ID NO:1 has the sequence MAKKLFITAALAAAVLA (SEQ ID NO:40). In other embodiments, signal peptides from other M. thermophila secreted proteins are used.

Still other signal peptides may be used, depending on the host cell and other factors. Effective signal peptide coding regions for filamentous fungal host cells include, but are not limited to, the signal peptide coding regions obtained from Aspergillus oryzae TAKA amylase, Aspergillus niger neutral amylase, Aspergillus niger glucoamylase, Rhizomucor miehei asparatic proteinase, Humicola insolens cellulase, Humicola lanuginosa lipase, and T. reesei cellobiohydrolase II (TrCBH2).

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis βlactamase, Bacillus stearothermophilus neutral proteases (nprT, nprS, nprM), and Bacillus subtilis prsA. Further signal peptides are described by Simonen and Palva, 1993, Microbiol Rev 57: 109-137 (incorporated herein by reference).

Useful signal peptides for yeast host cells also include those from the genes for Saccharomyces cerevisiae alpha-factor, Saccharomyces cerevisiae SUC2 invertase (see Taussig and Carlson, 1983, Nucleic Acids Res 11:1943-54; SwissProt Accession No. P00724), and others. See, e.g., Romanos et al., 1992, Yeast 8:423-488. Variants of these signal peptides and other signal peptides are suitable.

### Cellulose Binding Domains

Cellobiohydrolases and other cellulases generally have a multidomain structure comprising a catalytic domain (CD) and a cellulose binding domain (CBD) joined by a linker peptide. For example, the CBH2b of SEQ ID NO:1 comprises a CBD at amino acids 14-41 and a CD at amino acids 118-431. In some embodiments, a cellobiohydrolase variant of the present invention lacks a CBD. For example, in some embodiments the CBD of the cellobiohydrolase is cleaved from the catalytic domain following secretion of the enzyme. Alternatively, engineered cellobiohydrolases lacking a CBD may be used.

In some embodiments, a cellobiohydrolase variant of the present invention is truncated at the C-terminus and/or has a disruption of the cellulose binding domain (CBD). Without being bound to a particular theory, truncation of the C-terminus may disrupt the folding of the CBD and/or affect the ability of the CBD to bind substrate. Accordingly, in one aspect the present invention provides a CBH2b variant wherein the CBD, or a substantial portion of the CBD has been modified to disrupt folding and/or template binding. Such a modified CBD or deleted CBD is likely to beneficial for cellobiohydrolase properties, e.g., thermostability and/or tolerance for low pH.

In some embodiments, one or more modifications to the CBD is combined with one or more substitutions described herein (e.g., one or more amino acid substitutions or one or more substitution sets listed in Tables 3, 4, or 6).

The M. thermophila CBH2b CBD comprises residues 14-41 of SEQ ID NO:1. In some embodiments, a CBH2b variant of the present invention comprises the entire length of the CBD (optionally with the above-described modifications and optionally with other substitutions and/or modifications as described herein). In some embodiments, the CBH2b variant has an N-terminal deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 residues relative to SEQ ID NO:1. In some embodiments, the CBH2b variant has an N-terminal deletion of 1-10, 5-20, or 10-35 residues. In some embodiments, the CBH2b variant comprises a N-terminal deletion as described herein and further comprises one or more non-CBD residues appended to the N-terminus of the variant polypeptide.

CBDs may be homologous or heterologous to the catalytic domain. A homologous CBD is associated in the wild-type cellobiohydrolase with the parental catalytic domain. For example, the M. thermophila CBH2b CBD is homologous to the M. thermophila CBH2b catalytic domain. In some embodiments, a cellobiohydrolase variant of the present invention has multiple CBDs. The multiple CBDs can be in tandem or in different regions of the polypeptide.

### VII. Polynucleotides and Expression Systems Encoding Cellobiohydrolase Type 2 Variants

In another aspect, the present invention provides polynucleotides encoding the variant cellobiohydrolase polypeptides as described herein. The polynucleotide may be operably linked to one or more heterologous regulatory or control sequences that control gene expression to create a recombinant polynucleotide capable of expressing the polypeptide. Expression constructs containing a heterologous polynucleotide encoding the engineered cellobiohydrolase can be introduced into appropriate host cells to express the cellobiohydrolase.

In some embodiments, the cellobiohydrolase variant is generated from a wild-type cellobiohydrolase cDNA sequence (e.g., a wild-type M. thermophila CBH2b cDNA sequence, or a wild-type protein of Table 1) or the portion thereof comprising the open reading frame, with changes made as required at the codons corresponding to substitutions (residues mutated relative to the wild-type sequence as described herein, for example at Tables 3, 4, or 6). In addition, one or more of the “silent” nucleotide changes shown in Table 3 or 4 can be incorporated. A DNA sequence may also be designed for high codon usage bias codons (codons that are used at higher frequency in the protein coding regions than other codons that code for the same amino acid). The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. A codon whose frequency increases with the level of gene expression is typically an optimal codon for expression. In particular, a DNA sequence can be optimized for expression in a particular host organism. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; Codon W, John Peden, University of Nottingham; McInerney, J. O, 1998, Bioinformatics 14:372-73; Stenico et al., 1994, Nucleic Acids Res. 222437-46; Wright, F., 1990, Gene 87:23-29; Wada et al., 1992, Nucleic Acids Res. 20:2111-2118; Nakamura et al., 2000, Nucl. Acids Res. 28:292; Henaut and Danchin, “Escherichia coli and Salmonella,” 1996, Neidhardt, et al. Eds., ASM Press, Washington D.C., p. 2047-2066, all of which are incorporated herein be reference). The data source for obtaining codon usage may rely on any available nucleotide sequence capable of coding for a protein, e.g., complete protein coding sequences (CDSs), expressed sequence tags (ESTs), or predicted coding regions of genomic sequences.

Those having ordinary skill in the art will understand that due to the degeneracy of the genetic code, a multitude of nucleotide sequences encoding cellobiohydrolase polypeptides of the present invention exist. For example, the codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence. The invention contemplates and provides each and every possible variation of nucleic acid sequence encoding a polypeptide of the invention that could be made by selecting combinations based on possible codon choices.

Polynucleotides encoding cellobiohydrolases can be prepared using methods that are well known in the art. Typically, oligonucleotides of up to about 40 bases are individually synthesized, then joined (e.g., by enzymatic or chemical ligation methods, or polymerase-mediated methods) to form essentially any desired continuous sequence. For example, polynucleotides of the present invention can be prepared by chemical synthesis using, for example, the classical phosphoramidite method described by Beaucage, et al., 1981, Tetrahedron Letters, 22:1859-69, or the method described by Matthes, et al., 1984, EMBO J. 3:801-05, both of which are incorporated herein by reference. These methods are typically practiced in automated synthetic methods. According to the phosphoramidite method, oligonucleotides are synthesized, e.g., in an automatic DNA synthesizer, purified, annealed, ligated and cloned in appropriate vectors.

General texts that describe molecular biological techniques which are useful herein, including the use of vectors, promoters, protocols sufficient to direct persons of skill through in vitro amplification methods, including the polymerase chain reaction (PCR) and the ligase chain reaction (LCR), and many other relevant methods, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2009) (“Ausubel”), all of which are incorporated herein by reference. Reference is made to Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomeli et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564, all of which are incorporated herein by reference. Methods for cloning in vitro amplified nucleic acids are described in Wallace et al., U.S. Pat. No. 5,426,039, which is incorporated herein by reference.

### Vectors

The present invention makes use of recombinant constructs comprising a sequence encoding a cellobiohydrolase as described above. In a particular aspect the present invention provides an expression vector comprising a cellobiohydrolase polynucleotide operably linked to a heterologous promoter. Expression vectors of the present invention may be used to transform an appropriate host cell to permit the host to express the cellobiohydrolase protein. Methods for recombinant expression of proteins in fungi and other organisms are well known in the art, and a number expression vectors are available or can be constructed using routine methods. See, e.g., Tkacz and Lange, 2004, ADVANCES IN FUNGAL BIOTECHNOLOGY FOR INDUSTRY, AGRICULTURE, AND MEDICINE, KLUWER ACADEMIC/PLENUM PUBLISHERS. New York; Zhu et al., 2009, Construction of two Gateway vectors for gene expression in fungi Plasmid 6:128-33; Kavanagh, K. 2005, FUNGI: BIOLOGY AND APPLICATIONS Wiley, all of which are incorporated herein by reference.

Nucleic acid constructs of the present invention comprise a vector, such as, a plasmid, a cosmid, a phage, a virus, a bacterial artificial chromosome (BAC), a yeast artificial chromosome (YAC), and the like, into which a nucleic acid sequence of the invention has been inserted. Polynucleotides of the present invention can be incorporated into any one of a variety of expression vectors suitable for expressing a polypeptide. Suitable vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others. Any vector that transduces genetic material into a cell, and, if replication is desired, which is replicable and viable in the relevant host can be used.

In some embodiments, the construct further comprises regulatory sequences, including, for example, a promoter, operably linked to the protein encoding sequence. Large numbers of suitable vectors and promoters are known to those of skill in the art.

### Promoters

In order to obtain high levels of expression in a particular host it is often useful to express the cellobiohydrolase variant of the present invention under the control of a heterologous promoter. A promoter sequence may be operably linked to the 5′ region of the cellobiohydrolase coding sequence using routine methods.

Examples of useful promoters for expression of cellobiohydrolases include promoters from fungi. In some embodiments, a promoter sequence that drives expression of a gene other than a cellobiohydrolase gene in a fungal strain may be used. As a non-limiting example, a fungal promoter from a gene encoding an endoglucanase may be used. In some embodiments, a promoter sequence that drives the expression of a cellobiohydrolase gene in a fungal strain other than the fungal strain from which the cellobiohydrolase variant was derived may be used. As a non-limiting example, if the cellobiohydrolase variant is derived from M. thermophile, a promoter from a T. reesei cellobiohydrolase gene may be used or a promoter as described in WO 2010/107303, such as but not limited to the sequences identified as SEQ ID NO:25, SEQ ID NO:26, SEQ ID NO:27, SEQ ID NO:28, or SEQ ID NO:29 in WO 2010/107303.

Examples of other suitable promoters useful for directing the transcription of the nucleotide constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787, which is incorporated herein by reference), as well as the NA2-tpi promoter (a hybrid of the promoters from the genes for Aspergillus niger neutral alpha-amylase and Aspergillus oryzae triose phosphate isomerase), promoters such as cbh1, cbh2, egl1, egl2, pepA, hfb1, hfb2, xyn1, amy, and glaA (Nunberg et al., 1984, Mol. Cell Biol., 4:2306-2315, Boel et al., 1984, EMBO J. 3:1581-85 and EPA 137280, all of which are incorporated herein by reference), and mutant, truncated, and hybrid promoters thereof. In a yeast host, useful promoters can be from the genes for Saccharomyces cerevisiae enolase (eno-1), Saccharomyces cerevisiae galactokinase (gal1), Saccharomyces cerevisiae alcohol dehydrogenase/glyceraldehyde-3-phosphate dehydrogenase (ADH2/GAP), and S. cerevisiae 3-phosphoglycerate kinase. Other useful promoters for yeast host cells are described by Romanos et al., 1992, Yeast 8:423-488, incorporated herein by reference. Promoters associated with chitinase production in fungi may be used. See, e.g., Blaiseau and Lafay, 1992, Gene 120243-248 (filamentous fungus Aphanocladium album); Limon et al., 1995, Curr. Genet, 28:478-83 (Trichoderma harzianum), both of which are incorporated herein by reference.

In a yeast host, useful promoters can be from the genes for Saccharomyces cerevisiae enolase (ENO-1), Saccharomyces cerevisiae galactokinase (GALI), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8:423-88.

### Other Expression Elements

Cloned cellobiohydrolases may also have a suitable transcription terminator sequence, a sequence recognized by a host cell to terminate transcription. The terminator sequence is operably linked to the 3′ terminus of the nucleic acid sequence encoding the polypeptide. Any terminator that is functional in the host cell of choice may be used in the present invention.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary transcription terminators are described in U.S. Pat. No. 7,399,627, incorporated herein by reference.

Exemplary terminators for yeast host cells can be obtained from the genes for Saccharomyces cerevisiae enolase, Saccharomyces cerevisiae cytochrome C (CYCI), and Saccharomyces cerevisiae glyceraldehyde-3-phosphate dehydrogenase. Other useful terminators for yeast host cells are described by Romanos et al., 1992, Yeast 8:423-88.

A suitable leader sequence may be part of a cloned cellobiohydrolase sequence, which is a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

Sequences may also contain a polyadenylation sequence, which is a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are described by Guo and Sherman, Mol Cell Bio I5:5983-5990 (1995).

The expression vector of the present invention preferably contains one or more selectable markers, which permit easy selection of transformed cells. A selectable marker is a gene, the product of which provides for biocide or viral resistance, resistance to heavy metals, prototrophy to auxotrophs, and the like. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus. Suitable markers for yeast host cells are ADE2, HIS3, LEU2, LYS2, MET3, TRP1, and URA3.

### VIII. Host Cells Comprising Cellobiohydrolase Type 2 Variants

A vector comprising a sequence encoding a cellobiohydrolase is transformed into a host cell in order to allow propagation of the vector and expression of the cellobiohydrolase. In some embodiments, the cellobiohydrolase is post-translationally modified to remove the signal peptide and in some cases may be cleaved after secretion.

The transformed or transfected host cell described above is cultured in a suitable nutrient medium under conditions permitting the expression of the cellobiohydrolase. The medium used to culture the cells may be any conventional medium suitable for growing the host cells, such as minimal or complex media containing appropriate supplements. Cells are optionally grown in HTP media. Suitable media are available from commercial suppliers or may be prepared according to published recipes (e.g. in catalogues of the American Type Culture Collection).

### Expression Hosts

In some embodiments, the host cell is a eukaryotic cell. Suitable eukaryotic host cells include, but are not limited to, fungal cells, algal cells, insect cells, and plant cells. Suitable fungal host cells include, but are not limited to, Ascomycota, Basidiomycota, Deuteromycota, Zygomycota, Fungi imperfecti. Particularly preferred fungal host cells are yeast cells and filamentous fungal cells. The filamentous fungal host cells of the present invention include all filamentous forms of the subdivision Eumycotina and Oomycota. (Hawksworth et al., In Ainsworth and Bisby's Dictionary of The Fungi, 8th edition, 1995, CAB International, University Press, Cambridge, UK). Filamentous fungi are characterized by a vegetative mycelium with a cell wall composed of chitin, cellulose and other complex polysaccharides. The filamentous fungal host cells of the present invention are morphologically distinct from yeast.

In the present invention a filamentous fungal host cell may be a cell of a species of, but not limited to Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, Volvariella, or teleomorphs, or anamorphs, and synonyms, basionyms, or taxonomic equivalents thereof.

In some embodiments of the invention, the filamentous fungal host cell is of the Trichoderma species, e.g., T. longibrachiatum, T. viride (e.g., ATCC 32098 and 32086), Hypocrea jecorina or T. reesei (NRRL 15709, ATTC 13631, 56764, 56765, 56466, 56767 and RL-P37 and derivatives thereof; see Sheir-Neiss et al., Appl. Microbiol. Biotechnology, 20 (1984) pp 46-53), T. koningii, and T. harzianum. In addition, the term “Trichoderma” refers to any fungal strain that was previously classified as Trichoderma or currently classified as Trichoderma. In some embodiments of the invention, the filamentous fungal host cell is of the Aspergillus species, e.g., A. awamori, A. funigatus, A. japonicus, A. nidulans, A. niger, A. aculeatus, A. foetidus, A. oryzae, A. sojae, and A. kawachi. (Reference is made to Kelly and Hynes (1985) EMBO J. 4, 475479; NRRL 3112, ATCC 11490, 22342, 44733, and 14331; Yelton M., et al., (1984) Proc. Natl. Acad. Sci. USA, 81, 1470-1474; Tilburn et al., (1982) Gene 26, 205-221; and Johnston, I. L. et al. (1985) EMBO J. 4, 1307-1311). In some embodiments of the invention, the filamentous fungal host cell is of the Chtysosporium species, e.g., C. lucknowense, C. keratinophilum, C. tropicum, C. merdarium, C. inops, C. pannicola, and C. zonatum. In some embodiments of the invention, the filamentous fungal host cell is of the Myceliophthora species, e.g., M. thermophila. In some embodiments of the invention, the filamentous fungal host cell is of the Fusarium species, e.g., F. bactridioides, F. cerealis, F. crookwellense, F. culmorum, F. graminearum, F. graminum. F. oxysporum, F. roseum, and F. venenatum. In some embodiments of the invention, the filamentous fungal host cell is of the Neurospora species, e.g., N. crassa. Reference is made to Case, M. E. et al., (1979) Proc. Natl. Acad. Sci. USA, 76, 5259-5263; U.S. Pat. No. 4,486,553; and Kinsey, J. A. and J. A. Rambosek (1984) Molecular and Cellular Biology 4, 117-122. In some embodiments of the invention, the filamentous fungal host cell is of the Humicola species, e.g., H. insolens, H. grisea, and H. lanuginosa. In some embodiments of the invention, the filamentous fungal host cell is of the Mucor species, e.g., M. miehei and M. circinelloides. In some embodiments of the invention, the filamentous fungal host cell is of the Rhizopus species, e.g., R. oryzae and R. niveus. In some embodiments of the invention, the filamentous fungal host cell is of the Penicillum species, e.g., P. purpurogenum, P. chrysogenum, and P. verruculosum. In some embodiments of the invention, the filamentous fungal host cell is of the Thielavia species, e.g., T. terrestris and T. heterothallica. In some embodiments of the invention, the filamentous fungal host cell is of the Tolypocladium species, e.g., T. inflatum and T. geodes. In some embodiments of the invention, the filamentous fungal host cell is of the Trametes species, e.g., T. villosa and T. versicolor. In some embodiments of the invention, the filamentous fungal host cell is of the Sporotrichium species In some embodiments of the invention, the filamentous fungal host cell is of the Corynascus species.

In the present invention a yeast host cell may be a cell of a species of, but not limited to Candida, Hansenula, Saccharomyces, Schizosaccharomyces, Pichia, Kluyveromyces, and Yarrowia. In some embodiments of the invention, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, and Yarrowia lipolytica.

In some embodiments of the invention, the host cell is an algal cell such as Chlamydomonas (e.g., C. Reinhardtii) and Phormidium (P. sp. ATCC29409).

In other embodiments, the host cell is a prokaryotic cell. Suitable prokaryotic cells include gram positive, gram negative and gram-variable bacterial cells. For example and not for limitation, the host cell may be a species of Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia and Zymomonas. In some embodiments, the host cell is a species of Agrobacterium, Acinetobacter, Azobacter, Bacillus, Bifidobacterium, Buchnera, Geobacillus, Campylobacter, Clostridium, Corynebacterium, Escherichia, Enterococcus, Erwinia, Flavobacterium, Lactobacillus, Lactococcus, Pantoea, Pseudomonas, Staphylococcus, Salmonella, Streptococcus, Streptomyces, and Zymomonas.

In yet other embodiments, the bacterial host strain is non-pathogenic to humans. In some embodiments the bacterial host strain is an industrial strain. Numerous bacterial industrial strains are known and suitable in the present invention.

In some embodiments of the invention, the bacterial host cell is of the Agrobacterium species, e.g., A. radiobacter, A. rhizogenes, and A. rubi. In some embodiments of the invention the bacterial host cell is of the Arthrobacter species, e.g., A. aurescens, A. citreus, A. globformis, A. hydrocarboglutamicus, A. mysorens, A. nicotianae, A. paraffineus, A. protophonniae, A. roseoparqffinus, A. sulfureus, and A. ureafaciens. In some embodiments of the invention the bacterial host cell is of the Bacillus species, e.g., B. thuringensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. Some preferred embodiments of a Bacillus host cell include B. subtilis, B. licheniformis, B. megaterium, B. stearothermophilus and B. amyloliquefaciens. In some embodiments the bacterial host cell is of the Clostridium species, e.g., C. acetobutylicum, C. tetani E88, C. lituseburense, C. saccharobutylicum, C. perfringens, and C. beijerinckii. In some embodiments the bacterial host cell is of the Corynebacterium species e.g., C. glutamicum and C. acetoacidophilum. In some embodiments the bacterial host cell is of the Escherichia species, e.g., E. coli. In some embodiments the bacterial host cell is of the Erwinia species, e.g., E. uredovora, E. carotovora, E. ananas, E. herbicola, E. punctata, and E. terreus. In some embodiments the bacterial host cell is of the Pantoea species, e.g., P. citrea, and P. agglomerans. In some embodiments the bacterial host cell is of the Pseudomonas species, e.g., P. putida, P. aeruginosa, P. mevalonfi, and P. sp. D-0I 10. In some embodiments the bacterial host cell is of the Streptococcus species, e.g., S. equisimiles, S. pyogenes, and S. uberis. In some embodiments the bacterial host cell is of the Streptomyces species, e.g., S. ambofaciens, S. achromogenes, S. avermitilis, S. coelicolor, S. aureofaciens, S. aureus, S. fungicidicus, S. griseus, and S. lividans. In some embodiments the bacterial host cell is of the Zymomonas species, e.g., Z. mobilis, and Z. lipolytica.

Strains which may be used in the practice of the invention including both prokaryotic and eukaryotic strains, are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

Host cells may be genetically modified to have characteristics that improve protein secretion, protein stability or other properties desirable for expression and/or secretion of a protein. For example, knockout of Alp1 function results in a cell that is protease deficient. Knockout of pyr5 function results in a cell with a pyrimidine deficient phenotype. In particular embodiments host cells are modified to delete endogenous cellulase protein-encoding sequences or otherwise eliminate expression of one or more endogenous cellulases. In one embodiment expression of one or more endogenous cellulases is inhibited to increase production of cellulases of interest. Genetic modification can be achieved by genetic engineering techniques or using classical microbiological techniques, such as chemical or UV mutagenesis and subsequent selection. A combination of recombinant modification and classical selection techniques may be used to produce the organism of interest. Using recombinant technology, nucleic acid molecules can be introduced, deleted, inhibited or modified, in a manner that results in increased yields of cellobiohydrolase within the organism or in the culture. In one genetic engineering approach, homologous recombination can be used to induce targeted gene modifications by specifically targeting a gene in vivo to suppress expression of the encoded protein. In an alternative approach, siRNA, antisense, or ribozyme technology can be used to inhibit gene expression.

In some embodiments, the host cell for expression is a fungal cell (e.g., Myceliophthora thermophila) genetically modified to reduce the amount of endogenous cellobiose dehydrogenase (EC 1.1.3.4) and/or other enzyme (e.g., protease) activity that is secreted by the cell. A variety of methods are known in the art for reducing expression of protein in a cell, including deletion of all or part of the gene encoding the protein and site-specific mutagenesis to disrupt expression or activity of the gene product. See, e.g., Chaveroche et al., 2000, Nucleic Acids Research, 28:22 e97; Cho et al., 2006, MPMI 19: 1, pp. 7-15; Maruyama and Kitamoto, 2008, Biotechnol Lett 30:1811-1817; Takahashi et al., 2004, Mol Gen Genomics 272: 344-352; and You et al., 2009, Arch Micriobiol 191:615-622, the contents of each of which is incorporated by reference herein in its entirety. Random mutagenesis, followed by screening for desired mutations, can also be used. See e.g., Combier et al., 2003, FEMS Microbiol Lett 220:141-8 and Firon et al., 2003, Eukaryot Cell 2:247-55, incorporated by reference herein in its entirety.

Exemplary Myceliophthora thermophila cellobiose dehydrogenases are CDH1 (SEQ ID NO:34), encoded by the nucleotide sequence SEQ ID NO:33, and CDH2 (SEQ ID NO:36) encoded by the nucleotide sequence SEQ ID NO:35. The genomic sequence for the Cdh1 encoding CDH1 has accession number AF074951.1. In one approach, gene disruption is achieved using genomic flanking markers (see, e.g., Rothstein, 1983, Methods in Enzymology 101:202-11).

Site-directed mutagenesis may be used to target a particular domain, in some cases, to reduce enzymatic activity (e.g., glucose-methanol-choline oxido-reductase N and C domains of a cellobiose dehydrogenase or heme binding domain of a cellobiose dehydrogenase; see, e.g., Rotsaert et al., 2001, Arch. Biochem. Biophys. 390:206-14, which is incorporated by reference herein in its entirety).

In some embodiments, the cell is modified to reduce production of endogenous cellobiose dehydrogenases. In some embodiments, the cell is modified to reduce production of either CDH1 or CDH2. In some embodiments, the host cell has less than 75%, sometimes less than 50%, sometimes less than 30%, sometimes less than 25%, sometimes less than 20%, sometimes less than 15%, sometimes less than 10%, sometimes less than 5%, and sometimes less than 1% of the CDH1 and/or CDH2 activity of the corresponding cell in which the gene is not disrupted.

### Transformation and Culture

Introduction of a vector or DNA construct into a host cell can be effected by calcium phosphate transfection, DEAE-Dextran mediated transfection, PEG-mediated transformation, electroporation, or other common techniques (See Davis et al., 1986, Basic Methods in Molecular Biology, which is incorporated herein by reference).

The engineered host cells can be cultured in conventional nutrient media modified as appropriate for activating promoters, selecting transformants, or amplifying the cellobiohydrolase polynucleotide. Culture conditions, such as temperature, pH and the like, are those previously used with the host cell selected for expression, and will be apparent to those skilled in the art. As noted, many references are available for the culture and production of many cells, including cells of bacterial, plant, animal (especially mammalian) and archebacterial origin. See e.g., Sambrook, Ausubel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelli, et al., (1989) In Vitro Cell Dev. Biol. 25:1016-1024, all of which are incorporated herein by reference. For plant cell culture and regeneration, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); Jones, ed. (1984) Plant Gene Transfer and Expression Protocols, Humana Press, Totowa, N.J. and Plant Molecular Biology (1993) R. R. D. Croy, Ed. Bios Scientific Publishers, Oxford, U.K. ISBN 0 12 198370 6, all of which are incorporated herein by reference. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue (1998) from Sigma-Aldrich, Inc (St Louis, Mo.) (“Sigma-LSRCCC”) and, for example, The Plant Culture Catalogue and supplement (1997) also from Sigma-Aldrich, Inc (St Louis, Mo.) (“Sigma-PCCS”), all of which are incorporated herein by reference.

In some embodiments, cells expressing the cellobiohydrolase polypeptides of the invention are grown under batch or continuous fermentations conditions. Classical batch fermentation is a closed system, wherein the compositions of the medium is set at the beginning of the fermentation and is not subject to artificial alternations during the fermentation. A variation of the batch system is a fed-batch fermentation which also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

Cell-free transcription/translation systems can also be employed to produce cellobiohydrolase polypeptides using the polynucleotides of the present invention. Several such systems are commercially available. A general guide to in vitro transcription and translation protocols is found in Tymms (1995) In vitro Transcription and Translation Protocols: Methods in Molecular Biology, Volume 37, Garland Publishing, NY, which is incorporated herein by reference.

### IX. Production and Recovery of Cellobiohydrolase Type 2 Variants

In another aspect, the present invention is directed to a method of making a polypeptide having cellobiohydrolase activity. In some embodiments, the method comprises: providing a host cell transformed with any one of the described cellobiohydrolase polynucleotides of the present invention; culturing the transformed host cell in a culture medium under conditions in which the host cell expresses the encoded cellobiohydrolase polypeptide; and optionally recovering or isolating the expressed cellobiohydrolase polypeptide, or recovering or isolating the culture medium containing the expressed cellobiohydrolase polypeptide. The method further provides optionally lysing the transformed host cells after expressing the encoded cellobiohydrolase polypeptide and optionally recovering or isolating the expressed cellobiohydrolase polypeptide from the cell lysate. The present invention further provides a method of making an cellobiohydrolase polypeptide, said method comprising cultivating a host cell transformed with a cellobiohydrolase polypeptide under conditions suitable for the production of the cellobiohydrolase polypeptide and recovering the cellobiohydrolase polypeptide.

Typically, recovery or isolation of the cellobiohydrolase polypeptide is from the host cell culture medium, the host cell or both, using protein recovery techniques that are well known in the art, including those described herein. Cells are typically harvested by centrifugation, disrupted by physical or chemical means, and the resulting crude extract may be retained for further purification. Microbial cells employed in expression of proteins can be disrupted by any convenient method, including freeze-thaw cycling, sonication, mechanical disruption, or use of cell lysing agents, or other methods, which are well known to those skilled in the art.

The resulting polypeptide may be recovered/isolated and optionally purified by any of a number of methods known in the art. For example, the polypeptide may be isolated from the nutrient medium by conventional procedures including, but not limited to, centrifugation, filtration, extraction, spray-drying, evaporation, chromatography (e.g., ion exchange, affinity, hydrophobic interaction, chromatofocusing, and size exclusion), or precipitation. Protein refolding steps can be used, as desired, in completing the configuration of the mature protein. Finally, high performance liquid chromatography (HPLC) can be employed in the final purification steps. Purification of BGL1 is described in Parry et al., 2001, Biochem. J. 353:117, and Hong et al., 2007, Appl. Microbiol. Biotechnol. 73:1331, both incorporated herein by reference. In addition to the references noted supra, a variety of purification methods are well known in the art, including, for example, those set forth in Sandana (1997) Bioseparation of Proteins, Academic Press, Inc.; Bollag et al. (1996) Protein Methods, 2^{nd} Edition, Wiley-Liss, NY; Walker (1996) The Protein Protocols Handbook Humana Press, NJ; Harris and Angal (1990) Protein Purification Applications: A Practical Approach, IRL Press at Oxford, Oxford, England; Harris and Angal Protein Purification Methods: A Practical Approach, IRL Press at Oxford, Oxford, England; Scopes (1993) Protein Purification: Principles and Practice 3^{rd} Edition, Springer Verlag, NY; Janson and Ryden (1998) Protein Purification: Principles, High Resolution Methods and Applications, Second Edition, Wiley-VCH, NY; and Walker (1998) Protein Protocols on CD-ROM, Humana Press, NJ, all of which are incorporated herein by reference.

Immunological methods may be used to purify cellobiohydrolase polypeptides. In one approach, antibody raised against the cellobiohydrolase polypeptides (e.g., against a polypeptide comprising SEQ ID NO:1 or an immunogenic fragment thereof) using conventional methods is immobilized on beads, mixed with cell culture media under conditions in which the cellobiohydrolase is bound, and precipitated. In a related approach immunochromatography is used.

As noted, in some embodiments the cellobiohydrolase is expressed as a fusion protein including a non-enzyme portion. In some embodiments the cellobiohydrolase sequence is fused to a purification facilitating domain. As used herein, the term “purification facilitating domain” refers to a domain that mediates purification of the polypeptide to which it is fused. Suitable purification domains include metal chelating peptides, histidine-tryptophan modules that allow purification on immobilized metals, a sequence which binds glutathione (e.g., GST), a hemagglutinin (HA) tag (corresponding to an epitope derived from the influenza hemagglutinin protein; Wilson et al., 1984, Cell 37:767), maltose binding protein sequences, the FLAG epitope utilized in the FLAGS extension/affinity purification system (Immunex Corp, Seattle, Wash.), and the like. The inclusion of a protease-cleavable polypeptide linker sequence between the purification domain and the cellobiohydrolase polypeptide is useful to facilitate purification. One expression vector contemplated for use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography, as described in Porath et al., 1992, Protein Expression and Purification 3:263-281) while the enterokinase cleavage site provides a means for separating the cellobiohydrolase polypeptide from the fusion protein. pGEX vectors (Promega; Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

### X. Methods of Using Cellobiohydrolase Type 2 Variants

The cellobiohydrolase variants as described herein have multiple industrial applications, including but are not limited to, sugar production (e.g. glucose syrups), biofuels production, textile treatment, pulp or paper treatment, and applications in detergents or animal feed. A host cell containing a cellobiohydrolase variant of the present invention may be used without recovery and purification of the recombinant cellobiohydrolase, e.g., for use in a large scale biofermentor. Alternatively, the recombinant cellobiohydrolase variant may be expressed and purified from the host cell. The cellobiohydrolase variants of the present invention may also be used according to the methods of Section III (“Improved Saccharification Process”) of WO 2010/120557, the contents of which are incorporated by reference herein.

The variant cellobiohydrolases that have been described herein are particularly useful for breaking down cellulose to smaller oligosaccharides, disaccharides and monosaccharides. In some embodiments, the variant cellobiohydrolases are useful in saccharification methods. In some embodiments, the variant cellobiohydrolases may be used in combination with other cellulase enzymes including, for example, conventional enzymatic saccharification methods, to produce fermentable sugars.

Therefore, in one aspect the present invention provides a method of producing an end-product from a cellulosic substrate, the method comprising contacting the cellulosic substrate with a cellobiohydrolase variant as described herein (and optionally other cellulases) under conditions in which fermentable sugars are produced, and contacting fermentable sugars with a microorganism in a fermentation to produce the end-product. In some embodiments, the method further comprises pretreating the cellulosic substrate to increase its susceptibility to hydrolysis prior to contacting the cellulosic substrate with the cellobiohydrolase variant (and optionally other cellulases).

In some embodiments, enzyme compositions comprising the cellobiohydrolase variants of the present invention may be reacted with a biomass substrate in the range of about 25° C. to 100° C., about 30° C. to 90° C., about 30° C. to 80° C., and about 30° C. to 70° C. Also the biomass may be reacted with the cellobiohydrolase enzyme compositions at about 25° C., at about 30° C., at about 35° C., at about 40° C., at about 45° C., at about 50° C., at about 55° C., at about 60° C., at about 65° C., at about 70° C., at about 75° C., at about 80° C., at about 85° C., at about 90° C., at about 95° C. and at about 100° C. Generally the pH range will be from about pH 3.0 to 8.5, pH 3.5 to 8.5, pH 4.0 to 7.5, pH 4.0 to 7.0 and pH 4.0 to 6.5. The incubation time may vary for example from 1.0 to 240 hours, from 5.0 to 180 hrs and from 10.0 to 150 hrs. For example, the incubation time will be at least 1 hr, at least 5 hrs, at least 10 hrs, at least 15 hrs, at least 25 hrs, at least 50 hr, at least 100 hrs, at least 180 and the like. Incubation of the cellulase under these conditions and subsequent contact with the substrate may result in the release of substantial amounts of fermentable sugars from the substrate (e.g., glucose when the cellulase is combined with β-glucosidase). For example at least 20%, at least 30%, at least 40%, at least 50%, at least 60%, at least 70%, at least 80%, at least 90% or more fermentable sugar may be available as compared to the release of sugar by a wild-type polypeptide.

In some embodiments, an end-product of a fermentation is any product produced by a process including a fermentation step using a fermenting organism. Examples of end-products of a fermentation include, but are not limited to, alcohols (e.g., fuel alcohols such as ethanol and butanol), organic acids (e.g., citric acid, acetic acid, lactic acid, gluconic acid, and succinic acid), glycerol, ketones, diols, amino acids (e.g., glutamic acid), antibiotics (e.g., penicillin and tetracycline), vitamins (e.g., beta-carotine and B12), hormones, and fuel molecules other than alcohols (e.g., hydrocarbons).

In some embodiments, the fermentable sugars produced by the methods of the present invention may be used to produce an alcohol (such as, for example, ethanol, butanol, and the like). The variant cellobiohydrolases of the present invention may be utilized in any method used to generate alcohols or other biofuels from cellulose, and are not limited necessarily to those described herein. Two methods commonly employed are the separate saccharification and fermentation (SHF) method (see, Wilke et al., Biotechnol. Bioengin. 6:155-75 (1976)) or the simultaneous saccharification and fermentation (SSF) method disclosed for example in U.S. Pat. Nos. 3,990,944 and 3,990,945.

The SHF method of saccharification comprises the steps of contacting a cellulase with a cellulose containing substrate to enzymatically break down cellulose into fermentable sugars (e.g., monosaccharides such as glucose), contacting the fermentable sugars with an alcohol-producing microorganism to produce alcohol (e.g., ethanol or butanol) and recovering the alcohol. In some embodiments, the method of consolidated bioprocessing (CBP) can be used, where the cellulase production from the host is simultaneous with saccharification and fermentation either from one host or from a mixed cultivation.

In addition to SHF methods, a SSF method may be used. In some cases, SSF methods result in a higher efficiency of alcohol production than is afforded by the SHF method (Drissen et al., Biocatalysis and Biotransformation 27:27-35 (2009). One disadvantage of SSF over SHF is that higher temperatures are required for SSF than for SHF. In one embodiment, the present invention claims cellobiohydrolase polypeptides that have higher thermostability than a wild-type cellobiohydrolase and one practicing the present invention could expect an increase in ethanol production if using the cellulases described here in combination with SSF.

For cellulosic substances to be used effectively as substrates for the saccharification reaction in the presence of a cellulase of the present invention, it is often desirable to pretreat the substrate. Means of pretreating a cellulosic substrate are known in the art, including but not limited to chemical pretreatment (e.g., ammonia pretreatment, dilute acid pretreatment, dilute alkali pretreatment, or solvent exposure), physical pretreatment (e.g., steam explosion or irradiation), mechanical pretreatment (e.g., grinding or milling) and biological pretreatment (e.g., application of lignin-solubilizing microorganisms), and the present invention is not limited by such methods. Illustrative pretreatment procedures are described in further detail in the section describing lignocellulosic feedstocks.

Any alcohol producing microorganism such as those known in the art, e.g., Saccharomyces cerevisiae, can be employed with the present invention for the fermentation of fermentable sugars to alcohols and other end-products.

The fermentable sugars produced from the use of one or more cellobiohydrolase variants encompassed by the invention may be used to produce other end-products besides alcohols, such as but not limited to other biofuels compounds, acetone, an amino acid (e.g., glycine, lysine, and the like), organic acids (e.g., lactic acids and the like), glycerol, ascorbic acid, a diol (e.g., 1,3-propanediol, butanediol, and the like), vitamins, hormones, antibiotics, other chemicals, and animal feeds.

The cellobiohydrolase variants as described herein are further useful in the pulp and paper industry. In the pulp and paper industry, neutral cellulases can be used, for example, in deinking of different recycled papers and paperboards having neutral or alkaline pH, in improving the fiber quality, or increasing the drainage in paper manufacture. Other examples include, for example, the removal of printing paste thickener and excess dye after textile printing.

### Lignocellulosic Feedstocks

The term “lignocellulosic feedstock” refers to any type of plant biomass comprised of lignin and cellulose, such as, but not limited to, non-woody plant biomass, cultivated crops such as, but not limited to grasses, for example, but not limited to, C4 grasses, such as switch grass, cord grass, rye grass, miscanthus, reed canary grass, or a combination thereof, sugar processing residues, for example, but not limited to, baggase, such as sugar cane bagasse, beet pulp, or a combination thereof, agricultural residues, for example, but not limited to, soybean stover, corn stover, rice straw, sugar cane straw, rice hulls, barley straw, corn cobs, wheat straw, canola straw, oat straw, oat hulls, corn fiber, or a combination thereof, forestry biomass for example, but not limited to, recycled wood pulp fiber, sawdust, hardwood, for example aspen wood, softwood, or a combination thereof. Furthermore, the lignocellulosic feedstock may comprise cellulosic waste material or forestry waste materials such as, but not limited to, newsprint, cardboard and the like. Lignocellulosic feedstock may comprise one species of fiber or, alternatively, lignocellulosic feedstock may comprise a mixture of fibers that originate from different lignocellulosic feedstocks. In addition, the lignocellulosic feedstock may comprise fresh lignocellulosic feedstock, partially dried lignocellulosic feedstock, fully dried lignocellulosic feedstock, or a combination thereof.

Lignocellulosic feedstocks often comprise cellulose in an amount greater than about 20%, greater than about 30%, or greater than about 40% (w/w). For example, in some embodiments, the lignocellulosic material may comprise from about 20% to about 90% (w/w) cellulose, or any amount therebetween. Furthermore, in some additional embodiments, the lignocellulosic feedstock comprises lignin. In some embodiments, lignin is present in an amount greater than about 10% and is often present in an amount greater than about 15% (w/w). The lignocellulosic feedstock may also comprise small amounts of sucrose, fructose and starch. The lignocellulosic feedstock is generally first subjected to size reduction by methods including, but not limited to, milling, grinding, agitation, shredding, compression/expansion, or other types of mechanical action. Size reduction by mechanical action can be performed by any type of equipment adapted for the purpose, for example, but not limited to, hammer mills, tub-grinders, roll presses, refiners and hydrapulpers. In some embodiments, at least 90% by weight of the particles produced from the size reduction may have a length less than between about 1/16 and about 4 in. The measurement may be a volume or a weight average length.

The preferable equipment for the particle size reduction is a hammer mill or shredder. Subsequent to size reduction, the feedstock is typically slurried in water. This allows the feedstock to be pumped.

Lignocellulosic feedstocks of particle size less than about 6 inches may not require size reduction.

The feedstock may be slurried prior to pretreatment. In one embodiment of the invention, the consistency of the feedstock slurry is between about 2% and about 30% and more typically between about 4% and about 15%. Optionally, the slurry is subjected to a water or acid soaking operation prior to pretreatment.

Prior to pretreatment, the slurry may be dewatered using known methodologies to reduce steam and chemical usage. Examples of dewatering devices include pressurized screw presses, such as those described in WO 2010/022511 (incorporated herein by reference), pressurized filters and extruders.

A pretreated lignocellulosic feedstock, or pretreated lignocellulose, is a lignocellulosic feedstock that has been subjected to physical and/or chemical processes to make the fiber more accessible and/or receptive to the actions of cellulolytic enzymes.

The pretreatment may be carried out to hydrolyze the hemicellulose, or a portion thereof, that is present in the lignocellulosic feedstock to monomeric pentose and hexose sugars, for example xylose, arabinose, mannose, galactose, or a combination thereof. For example, the pretreatment may be carried out so that nearly complete hydrolysis of the hemicellulose and a small amount of conversion of cellulose to glucose occurs. During the pretreatment, typically an acid concentration in the aqueous slurry from about 0.02% (w/w) to about 2% (w/w), or any amount therebetween, is used for the treatment of the lignocellulosic feedstock. The acid may be, but is not limited to, hydrochloric acid, nitric acid, or sulfuric acid. For example, the acid used during pretreatment is sulfuric acid. One method of performing acid pretreatment of the feedstock is steam explosion using the process conditions set out in U.S. Pat. No. 4,461,648. Another method of pretreating the feedstock slurry involves continuous pretreatment, meaning that the lignocellulosic feedstock is pumped though a reactor continuously. Continuous acid pretreatment is familiar to those skilled in the art; see, for example, U.S. Pat. No. 7,754,457.

Pretreatment may also be conducted with alkali. In contrast to acid pretreatment, pretreatment with alkali may not hydrolyze the hemicellulose component of the feedstock. For example, the alkali may react with acidic groups present on the hemicellulose to open up the surface of the substrate. The addition of alkali may also alter the crystal structure of the cellulose so that it is more amenable to hydrolysis. Examples of alkali that may be used in the pretreatment include ammonia, ammonium hydroxide, potassium hydroxide, and sodium hydroxide.

An example of a suitable alkali pretreatment is Ammonia Freeze Explosion, Ammonia Fiber Explosion or Ammonia Fiber Expansion (“AFEX” process). According to this process, the lignocellulosic feedstock is contacted with ammonia or ammonium hydroxide in a pressure vessel for a sufficient time to enable the ammonia or ammonium hydroxide to alter the crystal structure of the cellulose fibers. The pressure is then rapidly reduced, which allows the ammonia to flash or boil and explode the cellulose fiber structure. (See U.S. Pat. Nos. 5,171,592; 5,037,663; 4,600,590; 6,106,888; 4,356,196; 5,939,544; 6,176,176; 5,037,663 and 5,171,592). The flashed ammonia may then be recovered according to known processes.

Dilute ammonia pretreatment utilizes more dilute solutions of ammonia or ammonium hydroxide than AFEX (see WO2009/045651 and US 2007/0031953). Such a pretreatment process may or may not produce any monosaccharides.

Yet a further non-limiting example of a pretreatment process for use in the present invention includes chemical treatment of the feedstock with organic solvents. Organic liquids in pretreatment systems are described by Converse et al. (U.S. Pat. No. 4,556,430; incorporated herein by reference), and such methods have the advantage that the low boiling point liquids easily can be recovered and reused. Other pretreatments, such as the Organosolv™ process, also use organic liquids (see U.S. Pat. No. 7,465,791, which is also incorporated herein by reference). Subjecting the feedstock to pressurized water may also be a suitable pretreatment method (see Weil et al. (1997) Appl. Biochem. Biotechnol. 68(1-2): 21-40, which is incorporated herein by reference).

The pretreated lignocellulosic feedstock may be processed after pretreatment by any of several steps, such as dilution with water, washing with water, buffering, filtration, or centrifugation, or a combination of these processes, prior to enzymatic hydrolysis, as is familiar to those skilled in the art.

The pretreatment produces a pretreated feedstock composition (e.g., a pretreated feedstock slurry) that contains a soluble component including the sugars resulting from hydrolysis of the hemicellulose, optionally acetic acid and other inhibitors, and solids including unhydrolyzed feedstock and lignin.

The soluble components of the pretreated feedstock composition may be separated from the solids to produce a soluble fraction. The soluble fraction, which includes the sugars released during pretreatment and other soluble components, including inhibitors, may then be sent to fermentation. It will be understood, however, that if the hemicellulose is not effectively hydrolyzed during the pretreatment, it may be desirable to include a further hydrolysis step or steps with enzymes or by further alkali or acid treatment to produce fermentable sugars. The foregoing separation may be carried out by washing the pretreated feedstock composition with an aqueous solution to produce a wash stream, and a solids stream comprising the unhydrolyzed, pretreated feedstock. Alternatively, the soluble component is separated from the solids by subjecting the pretreated feedstock composition to a solids-liquid separation, using known methods such as centrifugation, microfiltration, plate and frame filtration, cross-flow filtration, pressure filtration, vacuum filtration, and the like. Optionally, a washing step may be incorporated into the solids-liquids separation. The separated solids, which contain cellulose, may then be sent to enzymatic hydrolysis with cellulase enzymes in order to convert the cellulose to glucose.

The pretreated feedstock composition may be fed to the fermentation without separation of the solids contained therein. After the fermentation, the unhydrolyzed solids may be subjected to enzymatic hydrolysis with cellulase enzymes to convert the cellulose to glucose.

Prior to hydrolysis with cellulase enzymes, the pH of the pretreated feedstock slurry may be adjusted to a value that is amenable to the cellulase enzymes, which is typically between about 4 and about 6, although the pH can be higher if alkalophilic cellulases are used.

### Enzyme Mixtures

In another aspect, the invention provides an enzyme mixture that comprises a cellobiohydrolase variant polypeptide as described herein. In some embodiments, the enzymes of the enzyme mixture may be secreted from a host cell and in other embodiments, the enzymes of the enzyme mixture may not be secreted. The enzyme mixture may be cell-free, or in alternative embodiments, may not be separated from host cells that secrete an enzyme mixture component. A cell-free enzyme mixture typically comprises enzymes that have been separated from any cells. Cell-free enzyme mixtures can be prepared by any of a variety of methodologies that are known in the art, such as filtration or centrifugation methodologies. In certain embodiments, the enzyme mixture can be, for example, partially cell-free, substantially cell-free, or entirely cell-free. In some embodiments, one or more enzymes of the enzyme mixture are not secreted by the host cell. The cells may be lysed to release the enzyme(s). Enzymes may be recovered from the cell lysate or the cell lysate may be combined, with partial purification or without further purification, with the substrate.

The cellobiohydrolase variant and any additional enzymes present in an enzyme mixture may be secreted from a single genetically modified host cell or by different microbes in combined or separate fermentations. Similarly, the cellobiohydrolase variant and any additional enzymes present in the enzyme mixture may be expressed individually or in sub-groups from different strains of different organisms and the enzymes combined in vitro to make the enzyme mixture. It is also contemplated that the cellobiohydrolase variant and any additional enzymes in the enzyme mixture may be expressed individually or in sub-groups from different strains of a single organism, and the enzymes combined to make the enzyme mixture. In some embodiments, all of the enzymes are expressed from a single host organism, such as a genetically modified fungal cell.

In some embodiments, the enzyme mixture comprises other types of cellulases, selected from but not limited to cellobiohydrolase, endoglucanase, 8-glucosidase, and glycoside hydrolase 61 protein (GH61) cellulases. These enzymes may be wild-type or recombinant enzymes. In some embodiments, the cellobiohydrolase is a type 1 cellobiohydrolase, e.g., a T. reesei cellobiohydrolase I. In some embodiments, the endoglucanase comprises a catalytic domain derived from the catalytic domain of a Streptomyces avermitilis endoglucanase. See US 2010/0267089, incorporated herein by reference. In some embodiments, the at least one cellulase is derived from Acidothermus cellulolyticus, Thermobifida fusca, Humicola grisea, Myceliophthora thermophila, Chaetomium thermophilum, Acremonium sp., Thielavia sp, Trichoderma reesei, Aspergillus sp., or a Chtysosporium sp. Cellulase enzymes of the cellulase mixture work together resulting in decrystallization and hydrolysis of the cellulose from a biomass substrate to yield fermentable sugars, such as but not limited to glucose (See Brigham et al., 1995, in Handbook on Bioethanol (C. Wyman ed.) pp 119-141, Taylor and Francis, Washington D.C., which is incorporated herein by reference).

Cellulase mixtures for efficient enzymatic hydrolysis of cellulose are known (see, e.g., Viikari et al., 2007, “Thermostable enzymes in lignocellulose hydrolysis” Adv Biochem Eng Biotechnol 108:121-45, and US Pat. publications US 2009/0061484; US 2008/0057541; and US 2009/0209009 to Iogen Energy Corp.), each of which is incorporated herein by reference for all purposes. In some embodiments, mixtures of purified naturally occurring or recombinant enzymes are combined with cellulosic feedstock or a product of cellulose hydrolysis. Alternatively or in addition, one or more cell populations, each producing one or more naturally occurring or recombinant cellulases, may be combined with cellulosic feedstock or a product of cellulose hydrolysis.

In some embodiments, the enzyme mixture comprises an isolated CBH2b variant as described herein and at least one or more of an isolated cellobiohydrolase type 1a such as a CBH1a, an isolated endoglucanase (EG) such as a type 2 endoglucanase (EG2) or a type 1 endoglucanase (EG1) such as endoglucanase type 1b (EB1b), an isolated β-glucosidase (BGL), and an isolated glycoside hydrolase 61 protein (GH61). In some embodiments, at least 20%, at least 25%, at least 30%, at least 35%, at least 40%, at least 45%, or at least 50% of the enzyme mixture is a CBH2b variant. In some embodiments, the enzyme mixture further comprises a cellobiohydrolase type 1a (e.g., CBH1a), and the CBH2b variant and the CBH1a together comprise at least 25%, at least 30%, at least 35%, at least 40%, at least 45%, at least 50%, at least 55%, at least 60%, at least 65%, at least 70%, at least 75%, or at least 80% of the enzyme mixture. In some embodiments, the enzyme mixture further comprises a β-glucosidase (BGL), and the CBH2b variant, the CBH1a, and the BGL together comprise at least 30%, at least 35%, at least 40%, at least 45%, at least 50%, at least 55%, at least 60%, at least 65%, at least 70%, at least 75%, at least 80%, or at least 85% of the enzyme mixture. In some embodiments, the enzyme mixture further comprises an endoglucanase (EG), and the CBH2b variant, the CBH1a, the BGL, and the EG together comprise at least 35%, at least 40%, at least 45%, at least 50%, at least 55%, at least 60%, at least 65%, at least 70%, at least 75%, at least 80%, at least 85%, or at least 90% of the enzyme mixture. In some embodiments, the enzyme mixture comprises a CBH2b variant as described herein, a cellobiohydrolase type 1a (CBH1a), a β-glucosidase (BGL), an endoglucanase (EG), and a glycoside hydrolase 61 protein (GH61). In some embodiments, the enzyme mixture composition comprises isolated cellulases in the following proportions by weight (wherein the total weight of the cellulases is 100%): about 20%-10% of EG, about 20%-10% of BGL, about 30%-25% of CBH1a, about 10%-30% of GH61, and about 20%-25% of a CBH2b variant of the present invention. In some embodiments, the enzyme mixture composition comprises isolated cellulases in the following proportions by weight: about 20%-10% of EG, about 25%-15% of BGL, about 20%-30% of CBH1a, about 10%-15% of GH61, and about 25%-30% of a CBH2b variant of the present invention. In some embodiments, the enzyme mixture composition comprises isolated cellulases in the following proportions by weight: about 10%-15% of EG, about 20%-25% of BGL, about 30%-20% of CBH1a, about 15%-5% of GH61, and about 25%-35% of a CBH2b variant of the present invention. In some embodiments, the enzyme mixture composition comprises isolated cellulases in the following proportions by weight: about 15%-5% of EG, about 15%-10% of BGL, about 45%-30% of CBH1a, about 25%-5% of GH61, and about 40%-10% of a CBH2b variant of the present invention. In some embodiments, the enzyme mixture composition comprises isolated cellulases in the following proportions by weight: about 10% of EG, about 15% of BGL, about 40% of CBH1a, about 25% of GH61, and about 10% of a CBH2b variant of the present invention. In some embodiments, the enzyme mixture composition comprises isolated cellulases in the following proportions by weight: about 0% of EG, about 15%-10% of BGL, about 30%-40% of CBH1a, about 15%-10% of GH61, and about 30%-40% of a CBH2b variant of the present invention. In some embodiments, the enzyme component comprises more than 1 CBH2b variant (e.g. 2, 3 or 4 different CBH2b variants as disclosed herein). In some embodiments, an enzyme mixture composition of the invention can also contain one or more additional proteins such as those listed below. In some embodiments, an enzyme mixture composition of the invention can also contain one or more additional enzymes other than the EG, BGL, CBH1a, GH61, and/or CBH2b variant recited herein, such as the enzymes listed below. In some embodiments, an enzyme mixture composition of the invention can also contain one or more additional cellulases other than the EG, BGL, CBH1a, GH61, and/or CBH2b variant recited herein.

A cellobiohydrolase variant polypeptide of the invention may also be present in mixtures with non-cellulase enzymes that degrade cellulose, hemicellulose, pectin, and/or lignocellulose.

A “hemicellulase” as used herein, refers to a polypeptide that can catalyze hydrolysis of hemicellulose into small polysaccharides such as oligosaccharides, or monomeric saccharides. Hemicellullases include xylan, glucuonoxylan, arabinoxylan, glucomannan and xyloglucan. Hemicellulases include, for example, the following: endoxylanases, β-xylosidases, α-L-arabinofuranosidases, a-D-glucuronidases, feruloyl esterases, coumarolyl esterases, α-galactosidases, β-galactosidases, β-mannanases, and β-mannosidases. An enzyme mixture may therefore comprise a cellobiohydrolase variant of the invention and one or more hemicellulases.

An endoxylanase (EC 3.2.1.8) catalyzes the endohydrolysis of 1,4-β-D-xylosidic linkages in xylans. This enzyme may also be referred to as endo-1,4-β-xylanase or 1,4-β-D-xylan xylanohydrolase. An alternative is EC 3.2.1.136, a glucuronoarabinoxylan endoxylanase, an enzyme that is able to hydrolyse 1,4 xylosidic linkages in glucuronoarabinoxylans.

A β-xylosidase (EC 3.2.1.37) catalyzes the hydrolysis of 1,4-β-D-xylans, to remove successive D-xylose residues from the non-reducing termini. This enzyme may also be referred to as xylan 1,4-β-xylosidase, 1,4-β-D-xylan xylohydrolase, exo-1,4-β-xylosidase, or xylobiase.

An α-L-arabinofuranosidase (EC 3.2.1.55) catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, arabinofuranosidase, polysaccharide alpha-L-arabinofuranosidase, alpha-L-arabinofuranoside hydrolase, L-arabinosidase and alpha-L-arabinanase.

An alpha-glucuronidase (EC 3.2.1.139) catalyzes the hydrolysis of an alpha-D-glucuronoside to D-glucuronate and an alcohol.

An acetylxylanesterase (EC 3.1.1.72) catalyzes the hydrolysis of acetyl groups from polymeric xylan, acetylated xylose, acetylated glucose, alpha-napthyl acetate, and p-nitrophenyl acetate.

A feruloyl esterase (EC 3.1.1.73) has 4-hydroxy-3-methoxycinnamoyl-sugar hydrolase activity (EC 3.1.1.73) that catalyzes the hydrolysis of the 4-hydroxy-3-methoxycinnamoyl (feruloyl) group from an esterified sugar, which is usually arabinose in “natural” substrates, to produce ferulate (4-hydroxy-3-methoxycinnamate). Feruloyl esterase is also known as ferulic acid esterase, hydroxycinnamoyl esterase, FAE-III, cinnamoyl ester hydrolase, FAEA, cinnAE, FAE-I, or FAE-II.

A coumaroyl esterase (EC 3.1.1.73) catalyzes a reaction of the form: coumaroyl-saccharide+H(2)O=coumarate+saccharide. The saccharide may be, for example, an oligosaccharide or a polysaccharide. This enzyme may also be referred to as trans-4-coumaroyl esterase, trans-p-coumaroyl esterase, p-coumaroyl esterase or p-coumaric acid esterase. The enzyme also falls within EC 3.1.1.73 so may also be referred to as a feruloyl esterase.

An α-galactosidase (EC 3.2.1.22) catalyzes the hydrolysis of terminal, non-reducing α-D-galactose residues in α-D-galactosides, including galactose oligosaccharides, galactomannans, galactans and arabinogalactans. This enzyme may also be referred to as melibiase.

A β-galactosidase (EC 3.2.1.23) catalyzes the hydrolysis of terminal non-reducing β-D-galactose residues in β-D-galactosides. Such a polypeptide may also be capable of hydrolyzing α-L-arabinosides. This enzyme may also be referred to as exo-(1->4)-β-D-galactanase or lactase.

A β-mannanase (EC 3.2.1.78) catalyzes the random hydrolysis of 1,4-β-D-mannosidic linkages in mannans, galactomannans and glucomannans. This enzyme may also be referred to as mannan endo-1,4-β-mannosidase or endo-1,4-mannanase.

A β-mannosidase (EC 3.2.1.25) catalyzes the hydrolysis of terminal, non-reducing β-D-mannose residues in β-D-mannosides. This enzyme may also be referred to as mannanase or mannase.

A glucoamylase (EC 3.2.1.3) is an enzyme which catalyzes the release of D-glucose from non-reducing ends of oligo- and poly-saccharide molecules. Glucoamylase is also generally considered a type of amylase known as amylo-glucosidase.

An amylase (EC 3.2.1.1) is a starch cleaving enzyme that degrades starch and related compounds by hydrolyzing the α-1,4 and/or α-1,6 glucosidic linkages in an endo- or an exo-acting fashion. Amylases include α-amylases (EC 3.2.1.1); β-amylases (3.2.1.2), amylo-amylases (EC 3.2.1.3), α-glucosidases (EC 3.2.1.20), pullulanases (EC 3.2.1.41), and isoamylases (EC 3.2.1.68). In some embodiments, the amylase is an α-amylase.

One or more enzymes that degrade pectin may also be included in an enzyme mixture that comprises a cellobiohydrolase variant of the invention. A pectinase catalyzes the hydrolysis of pectin into smaller units such as oligosaccharide or monomeric saccharides. An enzyme mixture may comprise any pectinase, for example an endo-polygalacturonase, a pectin methyl esterase, an endo-galactanase, a pectin acetyl esterase, an endo-pectin lyase, pectate lyase, alpha rhamnosidase, an exo-galacturonase, an exo-polygalacturonate lyase, a rhamnogalacturonan hydrolase, a rhamnogalacturonan lyase, a rhamnogalacturonan acetyl esterase, a rhamnogalacturonan galacturonohydrolase or a xylogalacturonase.

An endo-polygalacturonase (EC 3.2.1.15) catalyzes the random hydrolysis of 1,4-α-D-galactosiduronic linkages in pectate and other galacturonans. This enzyme may also be referred to as polygalacturonase pectin depolymerase, pectinase, endopolygalacturonase, pectolase, pectin hydrolase, pectin polygalacturonase, poly-α-1,4-galacturonide glycanohydrolase, endogalacturonase; endo-D-galacturonase or poly(1,4-α-D-galacturonide) glycanohydrolase.

A pectin methyl esterase (EC 3.1.1.11) catalyzes the reaction: pectin+nH₂O=n methanol+pectate. The enzyme may also been known as pectinesterase, pectin demethoxylase, pectin methoxylase, pectin methylesterase, pectase, pectinoesterase or pectin pectylhydrolase.

A endo-galactanase (EC 3.2.1.89) catalyzes the endohydrolysis of 1,4-β-D-galactosidic linkages in arabinogalactans. The enzyme may also be known as arabinogalactan endo-1,4-β-galactosidase, endo-1,4-β-galactanase, galactanase, arabinogalactanase or arabinogalactan 4-β-D-galactanohydrolase.

A pectin acetyl esterase catalyzes the deacetylation of the acetyl groups at the hydroxyl groups of GalUA residues of pectin.

An endo-pectin lyase (EC 4.2.2.10) catalyzes the eliminative cleavage of (1→4)-α-D-galacturonan methyl ester to give oligosaccharides with 4-deoxy-6-O-methyl-α-D-galact-4-enuronosyl groups at their non-reducing ends. The enzyme may also be known as pectin lyase, pectin trans-eliminase; endo-pectin lyase, polymethylgalacturonic transeliminase, pectin methyltranseliminase, pectolyase, PL, PNL or PMGL or (1→4)-6-O-methyl-α-D-galacturonan lyase.

A pectate lyase (EC 4.2.2.2) catalyzes the eliminative cleavage of (1→4)-α-D-galacturonan to give oligosaccharides with 4-deoxy-α-D-galact-4-enuronosyl groups at their non-reducing ends. The enzyme may also be known polygalacturonic transeliminase, pectic acid transeliminase, polygalacturonate lyase, endopectin methyltranseliminase, pectate transeliminase, endogalacturonate transeliminase, pectic acid lyase, pectic lyase, α-1,4-D-endopolygalacturonic acid lyase, PGA lyase, PPase-N, endo-α-1,4-polygalacturonic acid lyase, polygalacturonic acid lyase, pectin trans-eliminase, polygalacturonic acid trans-eliminase or (1→4)-α-D-galacturonan lyase.

An alpha rhamnosidase (EC 3.2.1.40) catalyzes the hydrolysis of terminal non-reducing α-L-rhamnose residues in α-L-rhamnosides or alternatively in rhamnogalacturonan. This enzyme may also be known as α-L-rhamnosidase T, α-L-rhamnosidase N or α-L-rhamnoside rhamnohydrolase.

An exo-galacturonase (EC 3.2.1.82) hydrolyzes pectic acid from the non-reducing end, releasing digalacturonate. The enzyme may also be known as exo-poly-α-galacturonosidase, exopolygalacturonosidase or exopolygalacturanosidase.

An exo-galacturonase (EC 3.2.1.67) catalyzes a reaction of the following type: (1,4-α-D-galacturonide)n+H2O=(1,4-α-D-galacturonide)n-i+D-galacturonate. The enzyme may also be known as galacturan 1,4-α-galacturonidase, exopolygalacturonase, poly(galacturonate) hydrolase, exo-D-galacturonase, exo-D-galacturonanase, exopoly-D-galacturonase or poly(1,4-α-D-galacturonide) galacturonohydrolase.

An exopolygalacturonate lyase (EC 4.2.2.9) catalyzes eliminative cleavage of 4-(4-deoxy-α-D-galact-4-enuronosyl)-D-galacturonate from the reducing end of pectate, i.e. de-esterified pectin. This enzyme may be known as pectate disaccharide-lyase, pectate exo-lyase, exopectic acid transeliminase, exopectate lyase, exopolygalacturonic acid-trans-eliminase, PATE, exo-PATE, exo-PGL or (1→4)-α-D-galacturonan reducing-end-disaccharide-lyase.

A rhamnogalacturonan hydrolyzes the linkage between galactosyluronic acid and rhamnopyranosyl in an endo-fashion in strictly alternating rhamnogalacturonan structures, consisting of the disaccharide [(1,2-alpha-L-rhamnoyl-(1,4)-alpha-galactosyluronic acid].

A rhamnogalacturonan lyase cleaves α-L-Rhap-(1→4)-α-D-GalpA linkages in an endo-fashion in rhamnogalacturonan by beta-elimination.

A rhamnogalacturonan acetyl esterase catalyzes the deacetylation of the backbone of alternating rhamnose and galacturonic acid residues in rhamnogalacturonan.

A rhamnogalacturonan galacturonohydrolase hydrolyzes galacturonic acid from the non-reducing end of strictly alternating rhamnogalacturonan structures in an exo-fashion. This enzyme may also be known as xylogalacturonan hydrolase.

An endo-arabinanase (EC 3.2.1.99) catalyzes endohydrolysis of 1,5-α-arabinofuranosidic linkages in 1,5-arabinans. The enzyme may also be know as endo-arabinase, arabinan endo-1,5-α-L-arabinosidase, endo-1,5-α-L-arabinanase, endo-α-1,5-arabanase; endo-arabanase or 1,5-α-L-arabinan 1,5-α-L-arabinanohydrolase.

One or more enzymes that participate in lignin degradation may also be included in an enzyme mixture that comprises a cellobiohydrolase variant of the invention. Enzymatic lignin depolymerization can be accomplished by lignin peroxidases, manganese peroxidases, laccases and cellobiose dehydrogenases (CDH), often working in synergy. These extracellular enzymes are often referred to as lignin-modifying enzymes or LMEs. Three of these enzymes comprise two glycosylated heme-containing peroxidases: lignin peroxidase (LIP); Mn-dependent peroxidase (MNP); and, a copper-containing phenoloxidase laccase (LCC).

Laccase:

Laccases are copper containing oxidase enzymes that are found in many plants, fungi and microorganisms. Laccases are enzymatically active on phenols and similar molecules and perform a one electron oxidation. Laccases can be polymeric and the enzymatically active form can be a dimer or trimer.

Mn-Dependent Peroxidase:

The enzymatic activity of Mn-dependent peroxidase (MnP) in is dependent on Mn2+. Without being bound by theory, it has been suggested that the main role of this enzyme is to oxidize Mn2+ to Mn3+. (Glenn et al. (1986) Arch. Biochem. Biophys. 251:688-696). Subsequently, phenolic substrates are oxidized by the Mn3+ generated.

Lignin Peroxidase:

Lignin peroxidase is an extracellular heme that catalyses the oxidative depolymerization of dilute solutions of polymeric lignin in vitro. Some of the substrates of LiP, most notably 3,4-dimethoxybenzyl alcohol (veratryl alcohol, VA), are active redox compounds that have been shown to act as redox mediators. VA is a secondary metabolite produced at the same time as LiP by ligninolytic cultures of P. chrysosporium and without being bound by a theory, has been proposed to function as a physiological redox mediator in the LiP-catalysed oxidation of lignin in vivo (Harvey, et al. (1986) FEBS Lett. 195, 242-246).

An enzymatic mixture comprising a cellobiohydrolase variant of the invention may further comprise at least one of the following: a protease or a lipase that participates in cellulose degradation.

“Protease” includes enzymes that hydrolyze peptide bonds (peptidases), as well as enzymes that hydrolyze bonds between peptides and other moieties, such as sugars (glycopeptidases). Many proteases are characterized under EC 3.4, and are suitable for use in the invention. Some specific types of proteases include, cysteine proteases including pepsin, papain and serine proteases including chymotrypsins, carboxypeptidases and metalloendopeptidases.

“Lipase” includes enzymes that hydrolyze lipids, fatty acids, and acylglycerides, including phosphoglycerides, lipoproteins, diacylglycerols, and the like. In plants, lipids are used as structural components to limit water loss and pathogen infection. These lipids include waxes derived from fatty acids, as well as cutin and suberin.

An enzyme mixture that comprises a cellobiohydrolase variant of the invention may also comprise at least one expansin or expansin-like protein, such as a swollenin (see Salheimo et al., Eur. J. Biochem. 269, 4202-4211, 2002) or a swollenin-like protein.

Expansins are implicated in loosening of the cell wall structure during plant cell growth. Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein contains an N-terminal Carbohydrate Binding Module Family 1 domain (CBD) and a C-terminal expansin-like domain. For the purposes of this invention, an expansin-like protein or swollenin-like protein may comprise one or both of such domains and/or may disrupt the structure of cell walls (such as disrupting cellulose structure), optionally without producing detectable amounts of reducing sugars.

An enzyme mixture that comprises a cellobiohydrolase variant of the invention may also comprise at least one of the following: a polypeptide product of a cellulose integrating protein, scaffoldin or a scaffoldin-like protein, for example CipA or CipC from Clostridium thermocellum or Clostridium cellulolyticum respectively. Scaffoldins and cellulose integrating proteins are multi-functional integrating subunits which may organize cellulolytic subunits into a multi-enzyme complex. This is accomplished by the interaction of two complementary classes of domain, i.e. a cohesion domain on scaffoldin and a dockerin domain on each enzymatic unit. The scaffoldin subunit also bears a cellulose-binding module that mediates attachment of the cellulosome to its substrate. A scaffoldin or cellulose integrating protein for the purposes of this invention may comprise one or both of such domains.

An enzyme mixture that comprises a cellobiohydrolase variant of the invention may also comprise at least one cellulose induced protein or modulating protein, for example as encoded by a cip1 or cip2 gene or similar genes from Trichoderma reesei (see Foreman et al., J. Biol. Chem. 278(34), 31988-31997, 2003).

An enzyme mixture that comprises a cellobiohydrolase variant of the invention may comprise a member of each of the classes of the polypeptides described above, several members of one polypeptide class, or any combination of these polypeptide classes.

### Cellobiohydrolase Compositions

The cellobiohydrolase variants of the present invention may be used in combination with other optional ingredients such as a buffer, a surfactant, and/or a scouring agent. A buffer may be used with a cellobiohydrolase of the present invention (optionally combined with other cellulases, including another cellobiohydrolase) to maintain a desired pH within the solution in which the cellobiohydrolase is employed. The exact concentration of buffer employed will depend on several factors which the skilled artisan can determine. Suitable buffers are well known in the art. A surfactant may further be used in combination with the cellobiohydrolases of the present invention. Suitable surfactants include any surfactant compatible with the cellobiohydrolase and, optionally, with any other cellulases being used. Exemplary surfactants include an anionic, a non-ionic, and ampholytic surfactants. Suitable anionic surfactants include, but are not limited to, linear or branched alkylbenzenesulfonates; alkyl or alkenyl ether sulfates having linear or branched alkyl groups or alkenyl groups; alkyl or alkenyl sulfates; olefinsulfonates; alkanesulfonates, etc. Suitable counter ions for anionic surfactants include, but are not limited to, alkali metal ions such as sodium and potassium; alkaline earth metal ions such as calcium and magnesium; ammonium ion; and alkanolamines having 1 to 3 alkanol groups of carbon number 2 or 3. Ampholytic surfactants include, e.g., quaternary ammonium salt sulfonates, and betaine-type ampholytic surfactants. Nonionic surfactants generally comprise polyoxyalkylene ethers, as well as higher fatty acid alkanolamides or alkylene oxide adduct thereof, and fatty acid glycerine monoesters. Mixtures of surfactants can also be employed as is known in the art.

The present invention may be practiced at effective amounts, concentrations, and lengths of time. An effective amount of cellobiohydrolase is a concentration of cellobiohydrolase sufficient for its intended purpose. For example, an effective amount of cellobiohydrolase within a solution may vary depending on whether the intended purpose is to use the enzyme composition comprising the cellobiohydrolase in a saccharification process, or for example a textile application such as stone-washing denim jeans. The amount of cellobiohydrolase employed is further dependent on the equipment employed, the process parameters employed, and the cellulase activity, e.g., a particular solution will require a lower concentration of cellobiohydrolase where a more active cellulase composition is used as compared to a less active cellulase composition. A concentration of cellobiohydrolase and length of time that an cellobiohydrolase will be in contact with the desired target further depends on the particular use employed by one of skill in the art, as is described herein.

One skilled in the art may practice the present invention using cellobiohydrolases in either aqueous solutions, or a solid cellobiohydrolase concentrate. When aqueous solutions are employed, the cellobiohydrolase solution can easily be diluted to allow accurate concentrations. A concentrate can be in any form recognized in the art including, but not limited to, liquids, emulsions, gel, pastes, granules, powders, an agglomerate, or a solid disk. Other materials can also be used with or placed in the cellulase composition of the present invention as desired, including stones, pumice, fillers, solvents, enzyme activators, and anti-redeposition agents depending on the intended use of the composition.

### XI. Examples

The following examples are offered to illustrate, but not to limit the claimed invention.

### Example 1

### Wild-Type M. thermophila Cellobiohydrolase Type 2b Gene Acquisition and Protein Sequencing

The M. thermophila CBH2b cDNA gene was cloned by PCR amplification from a cDNA library using vector-specific primers that flanked the inserts. Following isolation of the gene, the gene was sequenced. The sequenced mature (i.e., lacking the endogenous signal peptide MAKKLFITAALAAAVLA, SEQ ID NO:40) M. thermophila CBH2b protein is provided as SEQ ID NO:1. It was determined that the amino acid sequence encoded by the M. thermophila CBH2b gene differed from the previously published sequence (U.S. patent application Ser. No. 11/487,547, published as US 2007/0238155). Specifically, the sequencing results indicated the presence of a tryptophan residue (encoded by the codon TGG) at position 14 of the mature M. thermophila CBH2b protein, which is not present in the published sequence. This newly identified tryptophan residue is located at the N-terminus of the protein in the cellulose-binding domain, and while not being bound by a particular theory, it is believed the residue may be important for binding to cellulose.

The thermostability of the M. thermophila CBH2b protein identified herein was measured against the M. thermophila CBH2b protein of the previously published sequence by using an Avicel assay. The thermostability of the two M. thermophila cellobiohydrolase type 2b proteins was found to be comparable.

### Example 2

### Construction of Expression Vectors

For the Round 1 thermostability screen, the wild-type M. thermophila CBH2b cDNA gene disclosed in Example 1 was cloned for expression in Saccharomyces cerevisiae strain InvSc1, a commercially available strain (Invitrogen, Carlsbad, Calif.). For the Round 2 thermostability screen, a variant derived from the Round 1 screen, variant 81, was cloned for expression in the Saccharomyces cerevisiae strain InvSc1.

### Example 3

### Shake Flask Procedure

A single colony of S. cerevisiae containing a plasmid with the M. thermophila CBH2b, or variant, cDNA gene was inoculated into 1 mL Synthetic Defined-uracil (SD-ura) Broth (2 g/L synthetic Drop-out minus uracil w/o yeast nitrogen base (from United States Biological, Swampscott, Mass.), 5 g/L Ammonium Sulphate, 0.1 g/L Calcium Chloride, 2 mg/L Inositol, 0.5 g/L Magnesium Sulphate, 1 g/L Potassium Phosphate monobasic (KH₂PO₄), 0.1 g/L Sodium Chloride) containing 3% glucose. Cells were grown overnight (at least 24 hours) in an incubator at 37° C. with shaking at 250 rpm. The culture was then diluted into 50 mL Defined Expression Medium with extra amino acids (“DEMA Extra”) broth (20 g/L glucose, 6.7 g/L yeast nitrogen base without amino acids (SigmaY-0626), 5 g/L ammonium sulphate, 24 g/L amino acid mix minus uracil (United States Biological D9535); pH approximately 6.0) containing 1% galactose in a 250 mL baffled sterile shake flask and incubated at 37° C. for 72 hours. Cells were pelleted by centrifugation (4000 rpm, 15 min, 4° C.). The clear media supernatant containing the secreted M. thermophila cellobiohydrolase was collected and stored at −20° C. until used.

### Example 4

### High Throughput Production of M. thermophila Cellobiohydrolase Type 2 Variants in Microtiter Plates

Variations were introduced into cellobiohydrolase cDNA sequences resulting in the generation of plasmid libraries. The plasmid libraries containing variant cellobiohydrolase genes were transformed into S. cerevisiae. Transformants were plated on SD-ura agar plate containing 2% glucose. After incubation for at least 48 hours at 30° C., colonies were picked using a Q-bot® robotic colony picker (Genetix USA, Inc., Beaverton, Oreg.) into shallow, 96-well well microtiter plates containing pH adjusted 200 μL SD-ura media and 3% glucose. Cells were grown for 24 hours at 30° C. with shaking at 250 rpm and 85% humidity. 20 μL of this overnight culture was then transferred into 96-well microtiter plates (deep well) containing 380 μL DEMA Extra medium and 1% galactose. The plates were incubated at 3TC with shaking at 250 rpm and 85% humidity for 48 hours. The deep plates were centrifuged at 4000 rpm for 15 minutes and the clear media supernatant containing the secreted cellobiohydrolase was used for the high throughput assays.

### Example 5

### Assays to Determine Cellobiohydrolase Thermostability

Cellobiohydrolase thermostability may be determined by exposing the cellobiohydrolase to stress conditions of elevated temperature and/or low pH for an appropriate period of time and then determining residual cellobiohydrolase activity by an activity assay such as a cellulose assay.

The cellobiohydrolase was challenged by incubating under conditions of pH 4.5 and 67° C. for 1 hour or pH 4.5 and 65° C., 73° C., or 75° C. for 18, 4, or 2 hours, respectively. Following the challenge incubation, residual activity of the cellobiohydrolase was measured.

Residual cellobiohydrolase activity was determined using a cellulose assay, which used microcrystalline cellulose (Avicel, from Sigma) as a substrate. In a total volume of 385 μL, 85 μL buffered supernatant containing cellobiohydrolase enzyme before and after thermal challenge was added to 200 g/L Avicel in 150 mM sodium acetate buffer (pH 5) containing beta-glucosidase, which converts cellobiose to glucose. The reaction was incubated at 50° C. for 24 hours. Conversion of Avicel to glucose was measured using a GOPOD Assay. The reaction was incubated at 50° C. for 24 hours. Glucose production was measured by mixing 10 μl of the above reaction with 190 μl of GOPOD assay mix. The reactions were allowed to shake for 30 min at room temperature. Absorbance of the solution was measured at 510 nm to determine the amount of glucose produced in the original Avicel biotransformation reaction.

### Example 6

### Evaluation of Optimal M. thermophila Cellobiohydrolase Type 2 Thermostability

The wild-type M. thermophila CBH2b thermostability profile was investigated at different temperatures and pHs using cellulose (Avicel) as a substrate. The experimental and analytical procedures are described in Examples 5 and 7. Wild-type M. thermophila CBH2b was found to retain <10% of unchallenged enzyme activity within 6 hours of incubation at pH 4.5-5, 65-75° C.

### Example 7

### High Throughput Assays to Identify Thermostability Improved M. thermophila Cellobiohydrolase Type 2 Variants

The M. thermophila cellobiohydrolase libraries were screened in high throughput using a thermostability assay. In the thermostability assay, the HTP media supernatant samples containing M. thermophila cellobiohydrolase variant enzymes were pre-incubated at pH 4.5, temperature 67-75° C. for 1-18 hours. The residual enzyme activity with and without the thermal challenge was measured using a cellulose-based assay (substrate: 200 g/L Avicel; pH 5.0; temperature 50° C.; time: 24 hrs) as described in Example 5.

The residual activity was calculated using the formula:

% residual activity=100×(Activity of challenged samples/Activity of unchallenged samples)

Residual activities of the M. thermophila cellobiohydrolase variants were compared to that of the wild-type M. thermophila CBH2b or variant 81 to identify the thermostability improved variants.

### Example 8

### Improved Thermostability of Engineered M. thermophila Cellobiohydrolase Type 2 Variants—Round 1 Screen

Improved M. thermophila CBH2b variants were identified from the high throughput screening of various M. thermophila cellobiohydrolase variant libraries as described in Example 4. For the Round 1 screen (Table 3), the M. thermophila CBH2b of SEQ ID NO:1 was the reference protein. From the Round 1 screen, one of the improved variants from the round (variant 81, as shown in Table 3) was then selected as the reference protein for the Round 2 screen.

Tables 3a-d and 4a-d summarize the improvement in thermostabilities of certain M. thermophila cellobiohydrolase variants. These and other variants are encompassed by the present invention.

Tables 3a-d summarize the results of the Round 1 screen, which identified improved M. thermophila cellobiohydrolase variants derived from the wild-type M. thermophila CBH2b (SEQ ID NO:1). Libraries for generating and screening variants were generated by several means. The thermostability of the cellobiohydrolase variants were compared to the thermostability of the wild-type M. thermophila CBH2b of SEQ ID NO:1. Thermostability was assessed by determining residual enzyme activity on microcrystalline cellulose (Avicel, Sigma) after incubation at pH 4.5 and 67° C. for 1 hour. Thermostability is presented as fold increase over wild-type M. thermophila CBH2b (SEQ ID NO:1). Silent nucleotide changes are indicated with respect to the wild-type M. thermophila CBH2b sequence. Amino acid positions (e.g., “W289”) and changes (e.g., “W289S”) are relative to SEQ ID NO:1.

TABLE 3
Improved M. Thermophila CBH2b variants

| | | | Stability: Fold increase |
|---|---|---|---|
| | Amino acid changes over wild-type | Silent | over wild-type |
| Variant | M. thermophila CBH2b | nucleotide | M. thermophila CBH2b |
| Number | (SEQ ID NO: 1) | changes | (SEQ ID NO: 1) |
| SEQ ID | — | — | — |
| NO: 1 | | | |

Table 3a

| | | | |
|---|---|---|---|
| 1 | W289S | | +++ |
| 2 | W289C | | +++ |
| 3 | R429D | | ++ |
| 4 | S336N | | ++ |
| 5 | S339R | | ++ |
| 6 | R429N | | + |
| 7 | W289M | | + |
| 8 | S437P | | + |
| 9 | R429H | | + |
| 10 | D424Q, L436K | | + |
| 11 | S336H | | + |
| 12 | T425P | | + |
| 13 | S437G | | + |
| 14 | A1V, S359D | | + |
| 15 | S339W | | + |
| 16 | C27Y | | + |
| 17 | W292R | | + |
| 18 | H286Q | | + |
| 19 | T425R | | + |
| 20 | Y120H | | + |
| 21 | N325H | | + |
| 22 | V267E | | + |
| 23 | P363H | | + |
| 24 | L128H | | + |
| 25 | N251T | | + |
| 26 | T73A, I227K | | + |
| 27 | Q381L | | + |
| 28 | E301K | | + |
| 29 | T425K | | + |
| 30 | Y432W | | + |
| 31 | Q297P | g342a | + |
| 32 | A360T | | + |
| 33 | S339Q | | + |
| 34 | Q297R | | + |
| 35 | N245T | | + |
| 36 | I227Q | | + |
| 37 | W292H | | + |
| 38 | I227A | | + |
| 39 | S260K | | + |
| 40 | N327L | | + |
| 41 | V267K | | + |
| 42 | G403T | | + |
| 43 | S333F | | + |
| 44 | V267L | | + |
| 45 | I227H | | + |
| 46 | P363D | | + |
| 47 | S359K | | + |
| 48 | H126E | | + |
| 49 | G384T | | + |
| 50 | Q169R, F353I | | + |
| 51 | D424N | | + |
| 52 | P276T | | + |
| 53 | G311Q | | + |
| 54 | Q382R | | + |
| 55 | S426K | | + |
| 56 | P363V | | + |
| 57 | I227G | | + |
| 58 | W292P | | + |
| 59 | M250G | | + |
| 60 | Q151L | | + |
| 61 | Q169K | | + |
| 62 | H286S | | + |
| 63 | T459K | | + |
| 64 | Q272R | | + |
| 65 | E405G | | + |
| 66 | E405P | | + |
| 67 | Q441K, P464R | | + |
| 68 | N341V | | + |
| 69 | Q297K | | + |
| 70 | W292A, R397H | | + |
| 71 | A294R | | + |
| 72 | Q165P | | + |
| 73 | Q169L | | + |
| 74 | N295R | | + |
| 75 | Q297Y | | + |
| 76 | N251D | | + |

Table 3b

| | | | |
|---|---|---|---|
| 77 | D160P, S230P, A253P, A334P | | ++ |
| 78 | R64P, S78P, A99P, S230P, S336P, | c858t | ++ |
| | E405P, S437P | | |
| 79 | S230P, A328P, E405P, S437P | | ++ |
| 80 | E39P, R64P, S78P, A99P, A253T, | | ++ |
| | A328P | | |
| 81 | S230P, A253P, E405P, S437P | | ++ |
| 82 | Q37P, E39P, S78P, A99P, D119P, | | ++ |
| | S230P, A253P, A328P, S437P | | |
| 83 | D119P, A253P, E405P, S437P | | ++ |
| 84 | E39P, S78P, A99P, S230P, A253P, | | ++ |
| | E405P | | |
| 85 | R64P, A99P, S230P, A253P, S336P | t582c | ++ |
| 86 | R64P, S230P, A253P, S336P, E405P | | ++ |
| 87 | E39P, R64P, S78P, V92P, A253P, | | ++ |
| | E405P, S437P | | |
| 88 | S78P, D119P, S230P, A253P, E405P | | ++ |
| 89 | E39P, S230P, A253P, S336P, E405P | | ++ |
| 90 | E39P, R64P, V92P, A253P, S437P | | + |
| 91 | D119P, S230P, S437P | | + |
| 92 | S230P, A253P, E405P | t294c | + |
| 93 | R64P, S78P, V92P, A99P, D119P, | c561t | + |
| | D160P, S230P, A253P, S336P, | | |
| | E405P | | |
| 94 | Q8H, R64P, V92P, D119P, S230P, | | + |
| | A253P, A328P, E405P | | |
| 95 | E6G, R64P, S78P, D160P, S230P, | | + |
| | A253P, E405P | | |
| 96 | E39P, S78P, D119P, D160P, S230P, | | + |
| | A253P, E405P | | |
| 97 | E39P, S78P, D119P, D160P, S230P, | | + |
| | A328P, E405P, S437P | | |
| 98 | R64P, S78P, D119P, S230P, A328P | | + |
| 99 | E39P, R64P, S78P, A253P, S336P, | | + |
| | E405P | | |
| 100 | E39P, S78P, V92P, D119P, D160P, | | + |
| | S230P, S336P | | |
| 101 | R64P, V92P, S104I, D160P, S230P, | c474t, t498c | + |
| | S336P, E405P | | |
| 102 | R64P, A99P, D160P, S230P, A328P, | c630t | + |
| | S336P, E405P | | |
| 103 | E39P, R64P, V92P, D160P, A328P, | | + |
| | S336P, K390N, E405P, A428T, | | |
| | S437P | | |
| 104 | E39P, S78P, A99P, D160P, A253P, | | + |
| | A328P, S336P, E405P | | |
| 105 | E39P, R64P, D160P, A253P, S336P, | | + |
| | L357M, E405P | | |
| 106 | E39P, R64P, S78P, V92P, A99P, | | + |
| | D119P, A328P, S336P, E405P | | |
| 107 | T61A, G107D | | − |
| 108 | R64P, S78P, D119P, D160P, S336P | | − |
| 109 | R64P, V92P, A328P, E405P | | − |
| 110 | D119P, D160P, A328P, S336P, | | − |
| | E405P, S437P | | |
| 111 | E39P, R64P, S78P, V92P | | − |

Table 3c

| | | |
|---|---|---|
| 112 | P109C, A279C | +++ |
| 113 | A129C, Q451C | +++ |
| 114 | I159C, A221C | ++ |
| 115 | V247C, A299C | ++ |
| 116 | I159C, A184C, A221C | ++ |
| 117 | A304C, A360C | ++ |
| 118 | L128C, W449C | ++ |
| 119 | A284C, L319C | + |
| 120 | I219C, A269C | + |
| 121 | I207C, T261C | + |
| 122 | A300C, L356C | + |
| 123 | V267C, D309C | + |

Table 3d

| | | | |
|---|---|---|---|
| 124 | T100G, E301K, S336N | | ++++ |
| 125 | N20P, S336N, T459N | g1335a | ++++ |
| 126 | Y120H, Q169R, A253T | | ++++ |
| 127 | I227M, S336N | | ++++ |
| 128 | D119R, I227M, S336N, L356P, | | ++++ |
| | S359D | | |
| 129 | Q165P, I227M, S336N, S437G | g111a | +++ |
| 130 | R7S, Q297R, A360T, T459R | | +++ |
| 131 | N20P, Q165R, S336N, F465R | | +++ |
| 132 | Q165P, S336N | C1395t | +++ |
| 133 | S336N, P363H | g708a | +++ |
| 134 | I227M, S336T, S339W | | +++ |
| 135 | S101R, S336N, P363H | C1260t | +++ |
| 136 | S67R, S336N, G384T, N462H | | +++ |
| 137 | S101R, P180Q, S336N | | +++ |
| 138 | Q169R, Q297K A360T, T425R | | +++ |
| 139 | G311R, S336T, S426R | g1344a | +++ |
| 140 | N20P, I227Q, V267L, S336K, S437P, | C1080t | ++ |
| | P464Q | | |
| 141 | N20P, K271R, S336N, Q381L, | | ++ |
| | S423G | | |
| 142 | V13G, I227M, S336N, P363D | | ++ |
| 143 | Q165P, A360T | | ++ |
| 144 | I227Q, A360K, A428P | | ++ |
| 145 | Q169R, I227H, W292A, S336N, | | ++ |
| | P363D | | |
| 146 | N20P, A139P, Q169R, I227Q, S333T, | | + |
| | S437P | | |
| 147 | N20P, S333T, A360T, T459G | | + |
| 148 | T100N | | + |

Fold increase for stability is represented as follows:
− = less than 1.1 fold increase over M. thermophila cellobiohydrolase type 2b (SEQ ID NO: 1)
+ = 1.1 to 1.9 fold increase over M. thermophila cellobiohydrolase type 2b (SEQ ID NO: 1)
++ = 2.0 to 2.9 fold increase over M. thermophila cellobiohydrolase type 2b (SEQ ID NO: 1)
+++ = 3.0 to 5.0 fold increase over M. thermophila cellobiohydrolase type 2b (SEQ ID NO: 1)
++++ = greater than 5.0 fold increase over M. thermophila cellobiohydrolase type 2b (SEQ ID NO: 1)

### Example 9

### Improved Thermostability of Engineered M. thermophila Cellobiohydrolase Type 2 Variants—Round 2 Screen

Tables 4a-d summarize the results of the Round 2 screen, which identified improved M. thermophila CBH2b variants derived from variant 81 (SEQ ID NO:2). Libraries for generating and screening variants were generated by DNA shuffling techniques. The thermostability of the cellobiohydrolase variants were compared to the thermostability of variant 81. Thermostability was assessed by determining residual enzyme activity on microcrystalline cellulose (Avicel, from Sigma) after incubation at pH 4.5 and 65° C., 73° C., or 75° C. for 18, 4, or 2 hours, respectively. Thermostability is presented as fold increase over variant 81 (SEQ ID NO:2). Silent nucleotide changes are indicated with respect to the wild-type M. thermophila CBH2b sequence. Amino acid positions (e.g., “Y121”) and changes (e.g., “Y121R”) are relative to SEQ ID NO:1.

TABLE 4
Improved M. thermophila CBH2b variants

| | | Stability: Fold | Stability: Fold | Stability: Fold |
|---|---|---|---|---|
| | Amino acid changes over wild-type | increase over | increase over | increase over |
| Variant | M. thermophila CBH2b | SEQ ID NO: 2 | SEQ ID NO: 2 | SEQ ID NO: 2 |
| Number | (SEQ ID NO: 1) | (65° C., 18 hrs) | (73° C., 4 hrs) | (75° C., 2 hrs) |
| 81 (SEQ | S230P, A253P, E405P, | — | — | — |
| ID NO: 2) | S437P | | | |

Table 4a

| | | |
|---|---|---|
| 149 | Y121R, S230P, A253P, | + |
| | E405P, S437P | |
| 150 | S168T, S230P, A253P, | + |
| | E405P, S437P | |

Table 4b

| | | |
|---|---|---|
| 151 | R7S, T100G, Y120H, S230P, A253P, | + |
| | E301K, E405P, S437P, T459N | |
| 152 | T100G, Y120H, Q165R, I227M, S230P, | + |
| | A253P, S339Q, E405P, S437P, T459N | |
| 153 | Y120H, Q165R, S230P, A253P, S339Q, | + |
| | E405P, S437P, T459R | |
| 154 | R7S, T100G, Y120H, Q165R, S230P, | + |
| | A253P, E301K, S339Q, E405P, S437P, | |
| | T459N | |
| 155 | R7S, T100G, Y120H, Q165R, I227M, | + |
| | S230P, A253P, S339Q, E405P, S437P, | |
| | T459N | |
| 156 | T100G, Y120H, S230P, A253P, E301K, | + |
| | S339Q, E405P, S437P, T459N | |
| 157 | T100G, Y120H, S230P, A253P, S339Q, | + |
| | G395C, E405P, S437P, T459R | |
| 158 | Q165R, S230P, A253P, S339Q, E405P, | + |
| | S437P, T459R | |
| 159 | R7S, T100G, Y120H, Q165R, S230P, | + |
| | A253P, S339Q, E405P, S437P, T459N | |
| 160 | R7S, T100G, Y120H, Q165R, S230P, | + |
| | A253P, S339Q, E405P, S437P, T459N | |
| 161 | R7S, T100G, Y120H, Q165R, S230P, | + |
| | A253P, S339Q, E405P, S437P, T459N | |
| 162 | R7S, T100G, Y120H, S230P, A253P, | + |
| | S339Q, E405P, S437P, T459N | |
| 163 | R7S, Y120H, S230P, A253P, S339Q, | + |
| | E405P, S437P, T459N | |
| 164 | T100G, Y120H, Q165R, S230P, A253P, | + |
| | S339Q, E405P, S437P | |
| 165 | T100G, Y120H, Q165R, S230P, A253P, | + |
| | E405P, S437P, T459N | |
| 166 | T100G, Y120H, Q165R, S230P, A253P, | + |
| | E405P, | |
| | S437P T459N | |
| 167 | T100G, Y120H, S230P, A253P, E405P, | + |
| | S437P, T459N | |
| 168 | T100G, Y120H, S230P, A253P, E405P, | + |
| | S437P, T459N | |
| 169 | Q165R, S230P, A253P, E405P, S437P, | − |
| | T459N | |
| 170 | T100G, Y120H, S230P, A253P, S339Q, | − |
| | E405P, S437P, T459N | |
| 171 | R7S, T100G, Y120H, S230P, A253P, | − |
| | E405P, | |
| | S437P | |
| 172 | T100G, Q165R, S230P, A253P, E405P, | − |
| | S437P | |
| 173 | T100G, S230P, A253P, S339Q, E405P, | − |
| | S437P | |

Table 4c

| | | |
|---|---|---|
| 174 | S230P, A253P, A300C, A304C, L356C, | ++++ |
| | A360C, E405P, S437P | |
| 175 | I159C, A221C, S230P, A253P, A304C, | ++++ |
| | A360C, E405P, S437P | |
| 176 | S230P, A253P, A304C, A360C, E405P, | ++++ |
| | S437P | |
| 177 | T54I, S230P, A253P, A304C, A360C, | ++++ |
| | E405P, S437P | |
| 178 | I159C, A221C, S230P, A253P, A300C, | +++ |
| | L356C, E405P, S437P | |

Table 4d

| | | |
|---|---|---|
| 179 | R7S, S230P, P253S, E405P, S437P | +++ |
| 180 | R7S, W292L, S230P, A253P, E405P, | ++ |
| | S437P | |

Fold increase for stability is represented as follows:
− = less than 1.1 fold increase over cellobiohydrolase variant 81 (SEQ ID NO: 2)
+ = 1.1 to 1.9 fold increase over cellobiohydrolase variant 81 (SEQ ID NO: 2)
++ = 2.0 to 2.9 fold increase over cellobiohydrolase variant 81 (SEQ ID NO: 2)
+++ = 3.0 to 5.0 fold increase over cellobiohydrolase variant 81 (SEQ ID NO: 2)
++++ = greater than 5.0 fold increase over cellobiohydrolase variant 81 (SEQ ID NO: 2)

### Example 10

### Characterization of Thermostability of Selected M. thermophila Cellobiohydrolase Type 2 Variants

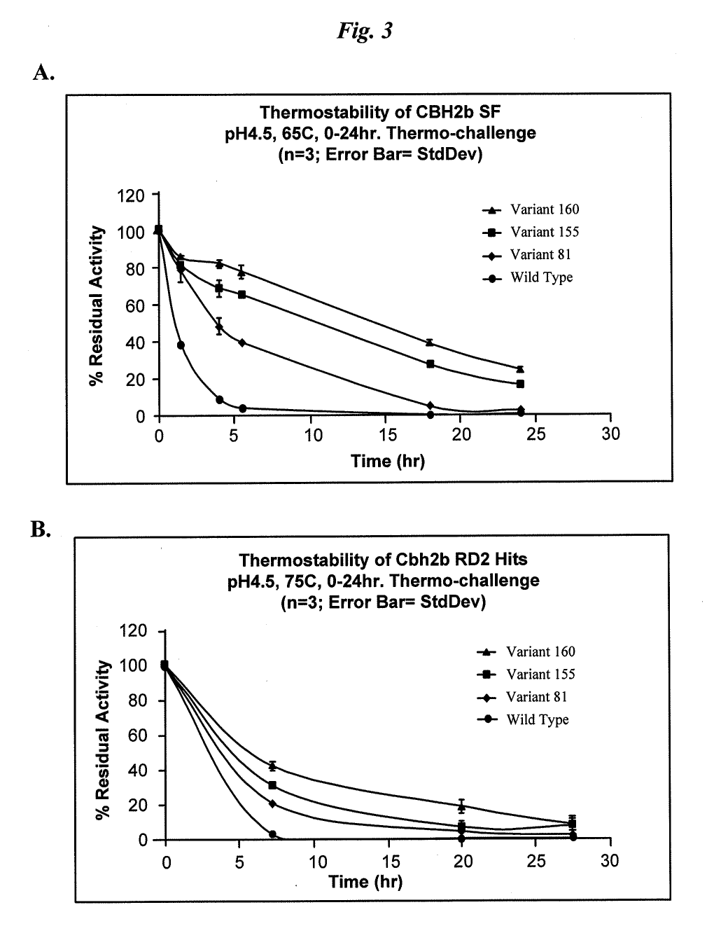

Three cellobiohydrolase variants (variant 81, variant 155, and variant 160) and wild-type M. thermophila CBH2b were grown in shake flask and characterized to determine their stability at low pH and high temperature. The samples containing various cellobiohydrolase variants or wild-type cellobiohydrolase were pre-incubated at pH 4.5, 65° C. or 75° C., for 0-24 hours. The residual enzyme activity after the thermal challenge was measured using Avicel (200 g/L) as a substrate in 450 mM sodium acetate buffer (pH 5). The reaction was incubated at 50° C. for 24 hours. FIGS. 3A and 3B illustrate the residual activity of improved cellobiohydrolase variants. Variants 155 and 160 were more stable than variant 81, while the wild-type cellobiohydrolase was the least stable under both conditions of pH 4.5, 65° C. and pH 4.5, 75° C.

### Example 11

### Performance Sensitive Positions Identified in M. thermophila Cellobiohydrolases Type 2a and 2b that Improve Protein Stability

To determine whether beneficial mutations at particular cellobiohydrolase residues impart improved stability across corresponding amino acid residues of multiple cellobiohydrolases, site-saturation mutagenesis was performed on both M. thermophila CBH2a and M. thermophila CBH2b. The wild-type M. thermophila CBH2a and CBH2b cDNA genes were expressed in S. cerevisiae as described in Example 2. Site-saturation libraries were constructed for each gene and HTP screens were performed according to the methods described in Example 6. Sequence analysis of the improved variants identified for CBH2a and CBH2b showed that there were multiple positions for which amino acid substitutions from the wild-type residue resulted in improvements in stability (as measured according to the methods of Examples 4 and 6) for corresponding positions in M. thermophila CBH2a and CBH2b (Table 5). Corresponding positions were identified by aligning CBH2a and CBH2b using the protein alignment tool Protein Align as shown in FIG. 1 and FIG. 2.

TABLE 5
Amino acid positions common to CBH2b and CBH2a
for mutations that improve protein stability

| CBH2b substitution | CBH2a substitution |
|---|---|
| S111N | Q31C |
| D119PR | N39ACKPRV |
| M250G | S160CLM |
| W289CMS | W199MT |
| A294R | A204QW |
| S336HKNPT | S250CPV |
| S359DK | S273AD |
| G384T | G297M |
| Y432W | S345EP |
| Q448K | T361EKQ |

### Example 12

### Improved Activity of Engineered M. thermophila High Throughput-Produced CBH2b Variants on Biomass Substrate

Activity on biomass substrate (wheat straw pretreated under acidic conditions) was measured using a buffered reaction mixture of 400 μl volume containing 20 mg of biomass, 100 μl of HTP produced CBH2b supernatant produced as described in Example 4, 200 μl of 1× filtrate (obtained from wheat straw pretreated under acidic conditions), and 100 μl of dilute M. thermophila β-glucosidase to produce 0.005 g/L final β-glucosidase concentration. The reactions were incubated at pH 5.0, 55° C. for 72 hours while shaking at 950 rpm. The reactions were centrifuged and 20 μl of the reaction was added to 180 μl of water.

Glucose was measured using a GOPOD-format assay (MEGAZYME, Ireland). From the diluted biomass hydrolysis reaction, 20 μl of reaction mixture was transferred to 180 μl of the GOPOD mixture (containing glucose oxidase, peroxidase and 4-aminoantipyrine) and incubated at room temperature for 30 minutes. The amount of glucose was measured spectrophotometrically at 510 nm with a Spectramax M2 (Molecular Devices, Sunnyvale, Calif.). The amount of glucose can be calculated based on the measured absorbance at 510 nm and using the standard curve. The glucose background was subtracted from data points using the background negative control. Background negative control was obtained by using media supernatant from cultures of S. cerevisiae cells without the CBH2b gene in the plasmid. The amount of glucose can be calculated based on the measured absorbance at 510 nm and using the standard curve. When wild-type M. thermophila CBH2b produced as described in Example 4 is used in the described reaction, approximately 1 g/L of glucose is produced.

Table 6 provides the relative cellobiohydrolase activity of M. thermophila CBH2b variants compared to M. thermophila wild-type CBH2b enzyme for glucose production. Relative activity is presented as fold improvement over M. thermophila wild-type CBH2b. The glucose background was subtracted from data points. Amino acid substitutions listed for each variant correspond to residue positions of M. thermophila wild-type CBH2b enzyme of SEQ ID NO:1.

TABLE 6
Yeast-produced M. thermophila CBH2b variant
polypeptides and relative cellobiohydrolase activity

| | | Relative |
|---|---|---|
| | Amino acid changes over | Improvement in |
| Variant | wild-type M. thermophila CBH2b without | Glucose |
| Number | signal peptide (SEQ ID NO: 1) | Production |
| WT | — | − |
| 1001 | H126M, Q165T, A212S, S339Q | + |
| 1002 | H126M, Q165T, Q169R, A212S, K271A, S339Q | + |
| 1003 | H126M, Q165T, Q169R, A212S, I227H, S339Q | + |
| 1004 | H126E, L128H, I227K | + |
| 1005 | Q165P, K271A, P340N, S359D | + |
| 1006 | P86T, Q165P, Q169R | + |
| 1007 | I227H, A253N, S359D | + |
| 1008 | Q165P, Q169R | + |
| 1009 | Q165P, Q169R, I227K, S359D | + |
| 1010 | P86T, H126M, Q165T, A212S, S339Q | + |
| 1011 | H126M, L128H, Q165T, A212S, S339Q | + |
| 1012 | H126M, Q165P, A212S, S339Q | + |
| 1013 | H126M, Q165T, P181A, A212S, S339Q | + |
| 1014 | H126M, Q165T, A212S, S339Q, Q382D | + |
| 1015 | H126M, L128H, Q165T, A212S, S339Q, Q382D | + |
| 1016 | P86T, H126M, Q165T, P181A, A212S, S339Q | + |
| 1017 | T33H, Q165P, Q169R | + |
| 1018 | A29T, Q165P, Q169R | + |
| 1019 | N20S, G107D, Q165P, Q169R | + |
| 1020 | N20L, Q165P, Q169R | + |
| 1021 | Q37H, Q165P, Q169R | + |
| 1022 | Q37F, Q165P, Q169R | + |
| 1023 | A29R, Q165P, Q169R | + |
| 1024 | A12I, Q165P, Q169R | + |
| 1025 | G112E, Q165P, Q169R | + |
| 1026 | T100V, Q165P, Q169R | + |
| 1027 | Q37L, T102W, S106W, Q165P, Q169R | + |
| 1028 | S101G, Q165P, Q169R | + |
| 1029 | L128E, Q165P, Q169R | + |
| 1030 | Y120R, Q165P, Q169R | + |
| 1031 | Y120N, Q165P, Q169R | + |
| 1032 | A139H, Q165P, Q169R | + |
| 1033 | A143M, Q165P, Q169R | + |
| 1034 | E146L, Q165P, Q169R | + |
| 1035 | D160H, Q165P, Q169R | + |
| 1036 | S142E, Q165P, Q169R | + |
| 1037 | I159S, Q165P, Q169R | ++ |
| 1038 | Q165P, Q169R, S339Q, S359D | + |
| 1039 | Q165P, Q169R, S339Q | +++ |
| 1040 | H126M, Q165T, Q169R, S339Q, S359D | ++ |
| 1041 | H126M, Q165T, Q169R, A253N, S339Q | + |
| 1042 | P86T, H126M, Q165P, Q169R, S339Q | + |
| 1043 | P86T, Q165P, S339Q | ++ |
| 1044 | P86T, H126M, Q165T, Q169R, K271A, S339Q | + |
| 1045 | H126M, Q165T, Q169R, S339Q | ++ |
| 1046 | H126M, Q165P, S339Q | ++ |
| 1047 | Q165P, Q169R, S339Q, S359D | +++ |
| 1048 | Q165P, Q169R, P340N | + |
| 1049 | Q165P, Q169R, S359Y | ++ |
| 1050 | H126M, Q165T, Q169R, A253N | ++ |
| 1051 | Q165P, Q169R | + |
| 1052 | W40L, P86T, Q165P, Q169R, K271A, S339L | + |
| 1053 | W40L, P86T, Q165P | ++ |
| 1054 | H126M, Q165P, Q169R, A253N, S339Q | +++ |
| 1055 | Q165P, Q169R, K271A | +++ |
| 1056 | P86T, H126L, Q165P | + |
| 1057 | P86T, Q165P, Q169R, S339Q | + |
| 1058 | Q165T, Q169R, K271A | + |
| 1059 | Q165P, Q169R, K271A, S339Q | ++ |
| 1060 | Q165P, Q169D | + |
| 1061 | M163L, Q165P, Q169R | + |
| 1062 | V164R, Q165P, Q169R | + |
| 1063 | Q165P, Q169R, A212L | ++ |
| 1064 | Q165P, Q169R, A176R | ++ |
| 1065 | Q165P, Q169R, N209S | + |
| 1066 | Q165P, Q169R, S206K | + |
| 1067 | Q165P, Q169R, G210A | + |
| 1068 | Q165P, Q169R, A212N, A364T | + |
| 1069 | Q165P, Q169R, A176G | + |
| 1070 | Q165P, Q169R, A212P | + |
| 1071 | Q165P, Q169R, A212R | + |
| 1072 | Q165P, Q169R, A213Q | + |
| 1073 | Q165P, Q169R, S206H, E228G | + |
| 1074 | Q165P, Q169R, A213H | + |
| 1075 | Q165P, Q169R, A213G | + |
| 1076 | Q165P, Q169R, A212C | + |
| 1077 | Q165P, Q169R, A360S | + |
| 1078 | Q165P, Q169R, S336L | + |
| 1079 | Q165P, Q169R, A360D | + |
| 1080 | Q165P, Q169R, S336E | + |
| 1081 | Q165P, Q169R, A360R | + |
| 1082 | Q165P, Q169R, A400V | + |
| 1083 | Q165P, Q169R, S336A | + |
| 1084 | Q165P, Q169R, A332S | + |
| 1085 | P87T, Q165P, Q169R, A212S | + |
| 1086 | P87T, Q165P, Q169R | + |
| 1087 | P87T, Q165P, Q169R, P340N | + |
| 1088 | P86T, S123R, Q165P, S168G, Q169R | + |
| 1089 | Q165P, S168R, Q169R, L356H | + |
| 1090 | Q165P, Q169R, L356G | ++ |
| 1091 | P86T, Q165P, S168R, Q169R, L356H, A428S | + |
| 1092 | Q165P, Q169R, L356H | + |
| 1093 | P86T, Q165P, Q169R, L356H | + |
| 1094 | P86T, Q165P, S168Q, Q169R, I227K, L356H | + |
| 1095 | P2S, P86T, Q165P, Q169R, I227K, L356H, A428S | + |
| 1096 | P86T, Q165P, S168G, Q169R, L356G | + |
| 1097 | P86T, Q165P, S168Q, Q169R, L356G | + |
| 1098 | P86T, Q165P, S168Q, Q169R, L356H, A428S | + |
| 1099 | P86T, Q165P, Q169R, L356G, A428S | ++ |
| 1100 | P86T, S123R, Q165P, S168G, Q169R, L356H | + |
| 1101 | Q165P, S168R, Q169R, I227K, L356H | + |
| 1102 | P86T, Q165P, Q169R, L356H, A428S | ++ |
| 1103 | P86T, Q165P, Q169R, L356G | + |
| 1104 | P86T, S123R, Q165P, S168Q, Q169R, L356H, A428S | + |
| 1105 | P86T, Q165P, S168Q, Q169R, L356G, A428S | + |
| 1106 | P86T, Q165P, S168G, Q169R | + |
| 1107 | P86T, Q165P, S168G, Q169R, L356H | + |
| 1108 | Q165P, S168R, Q169R, L356G | + |
| 1109 | Q165P, S168Q, Q169R, L356H | + |
| 1110 | Q165P, S168Q, Q169R, L356H, A428S | + |
| 1111 | P86T, Q165P, Q169R, L356G | + |
| 1112 | H126M, Q165P, Q169R, S339Q | ++ |
| 1113* | Q8P, H126M, Q165P, Q169R, S339Q | +++ |
| 1114 | Q165P, T166R, Q169R | ++ |
| 1115 | H126M, Q165P, Q169R, L356G | ++ |
| 1116 | Q165P, Q169R, S339Q | ++ |
| 1117 | H126M, Q165P, Q169R | ++ |
| 1118 | Q165P, Q169R, A212S, S339Q | + |
| 1119 | Q165P, Q169R, P181A, S339Q | + |
| 1120 | Q165P, T166R, Q169R, S339Q | + |
| 1121 | H126M, Q165P, Q169R, A212S, S339Q | + |
| 1122 | Q165P, Q169R, P181A | + |
| 1123 | H126M, Q165P, Q169R, P181A | + |
| 1124 | H126M, Q165P, Q169R, A212S | + |
| 1125 | H126M, Q165P, Q169R, P181A, S339Q | ++ |
| 1126 | Q165P, Q169R, S339Q, L356G | + |
| 1127 | Q165P, Q169R, A212S, S339Q, L356G | + |
| 1128 | V92K, Q165P, Q169R, S339Q, S359D | + |
| 1129 | V92R, Q165P, Q169R | ++ |
| 1130 | V92K, Q165P, Q169R, K224E, S359D | + |
| 1131 | V92K, Q165P, Q169R, S339Q, S359D | ++ |
| 1132 | Q165P, Q169R, S359D | + |
| 1133 | V92R, Q165P, Q169R, S339Q, S359D | + |
| 1134 | V92K, Q165P, Q169R, S339Q, S359D | ++ |
| 1135 | V92K, Q165P, Q169R, S359D | ++ |
| 1136 | V92R, M133F, Q165P, Q169R, S339Q | + |
| 1137 | Q165P, Q169R, S359D, R365L | + |
| 1138 | V92R, M133F, Q165P, Q169R | + |
| 1139 | V92R, M133F, Q165P, Q169R, S339Q | + |
| 1140 | V92K, M133V, Q165P, Q169R, S359D | + |
| 1141 | V92R, Q165P, Q169R, S339Q | + |
| 1142 | V92K, Q165P, Q169R | + |
| 1143 | V92K, Q165P, Q169R, S339Q | + |
| 1144 | V92R, M133F, Q165P, Q169R, S339Q, S359D | + |
| 1145 | V92R, M133F, Q165P, Q169R | + |
| 1146 | V92K, Q165P, Q169R, S339Q | + |
| 1147 | V92K, M163A, Q165P, Q169R, S339Q, S359D | + |
| 1148 | V92K, Q165P, Q169R, K224E | + |
| 1149 | V92R, M133V, Q165P, Q169R, S339Q | + |
| 1150 | S67G, T102C, V157S, Q165P, Q169R, V396R | + |
| 1151 | V157D, Q165P, Q169R, V396R | + |
| 1152 | S67G, V157D, Q165P, Q169R | + |
| 1153 | S67G, T102C, Q165P, Q169R, V396R | + |
| 1154 | T102C, V157S, Q165P, Q169R | + |
| 1155 | S67G, V157D, Q165P, Q169R, V396R | + |
| 1156 | V50K, Q165P, T166R, Q169R | ++ |
| 1157 | V50K, A117T, Q165P, Q169R | ++ |
| 1158 | V50R, Q165P, T166R, Q169R | + |
| 1159 | V50K, Q165P, Q169R | ++ |
| 1160 | Q165P, Q169R, S230P | + |
| 1161 | A117T, Q165P, Q169R | + |
| 1162 | V50R, A117T, Q165P, T166R, Q169R | + |
| 1163 | V50R, A117T, Q165P, Q169R | + |
| 1164 | V50R, Q165P, Q169R | + |
| 1165 | V50E, Q165P, Q169R, S230P, E405P | ++ |
| 1166 | V50E, Q165P, Q169R, E405P | + |
| 1167 | V50R, A117T, Q165P, T166R, Q169R, E405P | + |
| 1168 | E6N, A99V, Q165P, Q169R | ++ |
| 1169 | Q165P, Q169R, L436N | + |
| 1170 | Q165P, Q169R, A178N | + |
| 1171 | E6N, Q165P, Q169R, A253N | + |
| 1172 | Q165P, Q169R, A178N, A253N | + |
| 1173 | E6N, A99V, Q165P, Q169R, L436N | + |
| 1174 | E6N, A99V, Q165P, Q169R, A178N, A253N, L436N | + |
| 1175 | E6N, A99V, Q165P, Q169R, A178N | ++ |
| 1176 | E6N, Q165P, Q169R, A178N, L436N | ++ |
| 1177 | E6N, Q165P, Q169R | +++ |
| 1178 | Q165P, Q169R, A253N | ++ |
| 1179 | E6N, Q165P, Q169R | + |
| 1180 | A99V, Q165P, Q169R | + |
| 1181 | E6N, Q165P, Q169R, A178N | + |
| 1182 | T83D, P86T, H126M, Q165T, A212S, S339Q | + |
| 1183 | P86T, H126M, Q165T, A212S, S339Q, A360E | + |
| 1184 | P86T, H126M, Q165T, A212S, S339Q, P340N, R365G | +++ |
| 1185 | R74S, P86T, H126M, Q165T, A212S, S339Q | + |
| 1186 | G21D, P86T, H126M, Q165T, A212S, S339Q | + |
| 1187 | A36E, P86T, H126M, Q165T, A212S, S339Q | + |
| 1188 | P86T, H126M, Q165T, A212S, S339Q, L356H | + |
| 1189 | P86T, H126M, Q165T, A212S, S339Q, N358E | + |
| 1190 | P86T, H126M, Q165T, A212S, S339Q, A427T | + |
| 1191 | V50K, P86T, H126M, Q165T, A212S, S339Q | + |
| 1192 | P86T, Y120R, H126M, Q165T, A212S, S339Q | + |
| 1193 | P86T, H126M, Q165T, S168G, A212S, S339Q | + |
| 1194 | P86T, H126M, Q165T, A212S, S339Q, S359D, Q382D | + |
| 1195 | P86T, G107D, H126M, Q165T, A212S, S339Q | + |
| 1196 | P86T, H126M, Q165T, N179D, A212S, K224A, S339Q | + |
| 1197 | P86T, H126M, Q165T, A212S, S339Q, L356E | + |
| 1198 | P86T, H126M, Q165T, A212S, S339Q, S359D | + |
| 1199 | P86T, H126M, Q165T, Q169D, A212S, S339Q | + |
| 1200 | P86T, H126M, Q165T, N179D, A212S, S339Q | + |
| 1201 | V50E, P86T, H126M, Q165T, A212S, S339Q, S359Y, Q382H | + |
| 1202 | P86T, H126M, Q165T, A212S, K312A, S339Q | + |
| 1203 | P86T, P96E, H126M, Q165T, A212S, S339Q | + |
| 1204 | P86T, H126M, Q165T, S168Q, A212S, S339Q | + |
| 1205 | P86T, Y120N, H126M, Q165T, A212S, S339Q | + |
| 1206 | P86T, H126M, Q165P, A212S, S339Q | + |
| 1207 | L162I, Q165P, Q169R, I227K, S339E, S359D | + |
| 1208 | L162I, Q165P, Q169R, I227K, S359D | + |
| 1209 | L162I, Q165P, Q169R, I227K, S339E, S359D | +++ |
| 1210 | L162I, Q165P, Q169R, I227K, N249S, S359D, A360D | +++ |
| 1211 | Q165P, Q169R, I227K, S339E, S359D | + |
| 1212 | Q165P, Q169R, I227K, S339E, S359D, Q382D | +++ |
| 1213 | L162I, Q165P, Q169R, I227K, S339E, S359D, Q382D | +++ |
| 1214 | Q165P, Q169R, I227K, N249D, S359D, A360Q | + |
| 1215 | L128H, Q165P, Q169R, I227K, S359D, A360V | + |
| 1216 | R64C, L128H, Q165P, Q169R, A213G, I227K, S359D, A360V | + |
| 1217 | Y120E, Q165T, Q169R, I227K, S359D | + |
| 1218 | Q165P, Q169R, I227K, S339Q, N341D, S359D | + |
| 1219 | Q165P, Q169R, A212P, I227K, S359D | + |
| 1220 | P2H, Q165P, Q169R, I227K, T248S, S336N, S359D, A428N | + |
| 1221 | M163L, Q165P, Q169R, I227K, S359D | + |
| 1222 | S106Y, Q165P, Q169R, I227K, S336N, S359D | + |
| 1223 | Q165P, Q169R, A212R, I227K, S359D | + |
| 1224 | Q165P, Q169R, I227K, S336N, S359D | + |
| 1225 | Q151L, Q165P, Q169R, I227K, S336N, S359D | + |
| 1226 | M163L, Q165P, Q169R, I227K, S336N, S359D | + |
| 1227 | Q165P, Q169R, A212R, I227K, S336N, S359D, A428N | + |
| 1228 | Q165P, Q169R, A212R, I227K, S336N, S359D | + |
| 1229 | T161S, Q165P, Q169R, I227K, N249S, S339V, S359D | ++ |
| 1230 | Q165P, Q169R, I227K, V247A, S359D | ++ |
| 1231 | V113I, Q165P, Q169R, I227K, S359D | + |
| 1232 | Q165P, Q169R, I227K, S256R, S359D | + |
| 1233 | T161S, Q165P, Q169R, I227K, S359D | + |
| 1234 | T161S, Q165P, Q169R, I227K, S359D | + |
| 1235 | H126M, L128H, Q165P, Q169R, I227K, S339Q, S359D, Q382D | +++ |
| 1236 | R7H, Q165T, Q272H, S336N | +++ |
| 1237 | V92K, Q165T, S230P, A253T, S339Q | +++ |
| 1238 | V157S, Q165P, Q169R, S359D | +++ |
| 1239 | Q165P, Q169R, I227M, S359D | +++ |
| 1240 | L162I, Q165P, Q169R, I227K, S339E, S359D, Q382D | +++ |
| 1241 | L162I, Q165P, Q169R, I227K, S359D, A360D | +++ |
| 1242 | Q165P, Q169R, I227K, S339E, S359D, Q382D | +++ |
| 1243 | Q165P, Q169R, I227K, S359D | +++ |
| 1244 | R64C, Q165P, Q169R, I227M, S359D | +++ |
| 1245 | L162I, Q165P, Q169R, I227K, S339E, S359D | +++ |
| 1246 | Q165P, Q169R, I227K, V247A, S359D | +++ |
| 1247 | T161S, Q165P, Q169R, I227K, N249S, S339V, S359D | +++ |
| 1248 | Q165P, Q169R, I227M, F353L, S359D | +++ |
| 1249 | Q165P, Q169R, A212R, I227K, S336N, S359D | +++ |
| 1250 | S106Y, Q165P, Q169R, I227K, S336N, S359D | +++ |
| 1251 | Q165P, Q169R, I227K, S339E, S359D | +++ |
| 1252 | Q165P, Q169R, I227K, S339Q, S359D, Q382D | +++ |
| 1253 | H126M, Q165P, Q169R, I227K, S339Q, S359D, Q382D | +++ |
| 1254 | Q165P, Q169R, I227K, S359D, Q382D | +++ |
| 1255 | H126M, Q165P, Q169R, I227K, S359D, Q382D | +++ |
| 1256 | W14L, Q169R, I227K, S339Q, S359D | ++ |
| 1257 | V92S, Q165P, Q169R, I227K, S339E, S359D, N401D, | ++ |
| | E405P, S437P | |
| 1258 | Q165P, Q169R, S230P, A253T, S359D | ++ |
| 1259* | V50H, V92K, Q165T, S230P, S339Q, S359D | ++ |
| 1260 | Q165T, I227K, S359D | ++ |
| 1261 | V50K, Q165P, Q169R, A253T, S359D, V396E, E405P, S437P | ++ |
| 1262 | G18D, V92K, Q165P, Q169R, S230P, S339E, S359D | ++ |
| 1263 | V92K, Q165P, Q169R, S230P, A253T, S339E, S359D, Q382D | ++ |
| 1264 | Q165P, Q169R, S230P, A253T, N308E, S339Q | ++ |
| 1265 | V50K, Q165T, S230P, S260K, S339E, S359D, Q382A | ++ |
| 1266 | Q165P, Q169R, K224W, S339E, S359D, Q382D | ++ |
| 1267 | Q165P, Q169R, A212R, I227K, S336N, S359D, A428N | ++ |
| 1268 | R64C, Q165P, Q169R, I227T, S359D | ++ |
| 1269 | Q165P, Q169R, I227K, S336N, S359D | ++ |
| 1270 | M163L, Q165P, Q169R, I227K, S336N, S359D | ++ |
| 1271 | T161S, Q165P, Q169R, I227K, S359D | ++ |
| 1272 | Q151L, Q165P, Q169R, I227K, S336N, S359D | ++ |
| 1273 | Q49K, Q165P, Q169R, A176G, I227K, S359D | ++ |
| 1274 | R64C, Q165P, Q169R, I227K, S359D | ++ |
| 1275 | L162I, Q165P, Q169R, I227K, S359D | ++ |
| 1276 | Q165P, Q169D, I227K, S359D | ++ |
| 1277 | Q165P, Q169R, A176G, I227K, F353L, S359D | ++ |
| 1278 | Q165P, Q169R, N209S, I227K, S359D | ++ |
| 1279 | S94N, Q165P, Q169R, I227M, S359D | ++ |
| 1280 | Q165P, Q169R, I227K, S359D, A360V | ++ |
| 1281 | Q165P, Q169R, A212R, I227K, S359D | ++ |
| 1282 | Q165P, Q169R, I227K, N249D, S359D, A360Q | ++ |
| 1283 | Q37H, Q165P, Q169R, I227K, S359D | ++ |
| 1284 | Q165P, Q169R, A212S, I227K, S339Q, S359D, Q382D | + |
| 1285 | P86T, Q165P, Q169R, I227K, S339Q, S359D | + |
| 1286 | P56T, Q165T, Q169R, I227K, S359D, Q382D | + |
| 1287 | Q165P, Q169R, A212S, I227K, A253T, S359D, Q382D | + |
| 1288 | H126M, L128H, Q165P, Q169R, I227K, S359D, Q382D | + |
| 1289 | Q165P, Q169R, I227K, S339Q, S359D | + |
| 1290 | Q165T, Q169R, I227K, S339Q, S359D | + |
| 1291 | P86T, Q165T, Q169R, I227K, S339Q, S359D | + |
| 1292 | Q165T, Q169R, I227K, S339Q, S359D, Q382D | + |
| 1293 | Q165P, Q169R, A212S, I227K, S359D | + |
| 1294 | S81P, P86T, Q165P, Q169R, I227K, S359D | + |
| 1295 | V157H, Q165T, S230P, A253P, S339E, S359D, Q382D | + |
| 1296 | V50E, V157S, Q165T, S359D | + |
| 1297 | V164E, Q165T, S339E, S359D, Q382D | + |
| 1298 | Y120N, Q165P, Q169R, S230P, A253P, S359D, Q382D | + |
| 1299 | V92K, Q165P, Q169R, S339Q, S359D, Q382D | + |
| 1300 | V50E, Q165P, Q169R, S230P, A253P, S336N | + |
| 1301 | V157D, Q165T, S230P, V252N, A253P, S359D, E405P, | + |
| | S437P, Q448T | |
| 1302 | I95H, L128H, V157S, Q165T, V267L, S339Q, S359D, S437P | + |
| 1303 | V157D, Q165P, Q169R, S230P, V252N, A253P, S339E, | + |
| | S359D, Q382D | |
| 1304 | V50H, L162I, Q165P, Q169R, S230P, S339E, S359D, | + |
| | V396E, E405P, S437P | |
| 1305 | L128H, K224E, S230P, A253P, S339E, S359D, Q382D, | + |
| | V396E, E405P, S437P | |
| 1306 | R7S, Q165P, Q169R, I227K, Q272H, S359D | + |
| 1307 | V164E, Q165T, S339E, S359D | + |
| 1308 | R7S, V50K, V157D, Q165T, I227K, S339Q, S359D | + |
| 1309 | T61A, V164E, Q165T, S230P, V252N, A253P, S339E, S359D | + |
| 1310 | V50R, A139T, V157D, Q165T, I227K, S359D | + |
| 1311 | V50H, V92S, Q165P, Q169R, I227K, S359D | + |
| 1312 | V50K, V92S, Q165P, Q169R, I227K, S359D, Q382A | + |
| 1313 | V92S, Q165T, S230P, V252N, A253P, S339E, S359D, | + |
| | Q382D, N401D, E405P, S437P | |
| 1314 | V157H, Q165T, I227K, S339E, S359D, Q382D | + |
| 1315 | V157D, Q165T, S230P, S359D, S437P | + |
| 1316 | Q8L, V50D, A99V, Q165P, Q169R, I227K, S339E, S359D, | + |
| | Q382D, N401D, E405P, S437P | |
| 1317 | L162I, Q165P, Q169R, Q297R, A360T, E405P, S437P | + |
| 1318 | R7S, V92D, V157D, Q165T, S230P, A253P, S339E, S359D | + |
| 1319 | V50D, V164E, Q165T, S230P, S359D, H404N | + |
| 1320 | V92K, Q165T, I227K, T459R | + |
| 1321 | V50K, V157D, Q165T, S230P, S339Q, Q382A, | + |
| | N401D, E405P, S437P | |
| 1322 | R7S, Q165T, S230P, V252N, A253P, S339E, S359D, Q382D | + |
| 1323 | V92D, Q165T, S339E, S359D, Q382D, E405P, S437P | + |
| 1324 | N47K, Q165P, Q169R, S230P, S359D, E405P | + |
| 1325 | Q8L, V92D, Q165T, I227A, S336N | + |
| 1326 | Q165P, Q169R, I227K, S339E, S359D, Q382D, E405Q | + |
| 1327 | V92S, Q165P, Q169R, S230P, S359D | + |
| 1328 | Y120R, Q165T, I227K, S339E, S359D, Q382D | + |
| 1329 | V50R, V164R, Q165P, Q169R, S359D | + |
| 1330 | Q165T, I227K, V252N, A253P, S339Q, S359D, Q382A, E405P | + |
| 1331 | V50K, Q165T, S230P, A253P, S359D, V396E, E405P, S437P | + |
| 1332 | V50H, V157H, Q165T, Q272H, S359D, V396E, E405P, S437P | + |
| 1333 | V157S, Q165T, I227K, Q272H, S359D | + |
| 1334 | Q165T, I227K, S359D, Q382D | + |
| 1335* | I95N, L162I, Q165P, Q169R, S230P, A253P, S359D | + |
| 1336 | Q165P, Q169R, I227K, S359D, S437P | + |
| 1337 | I95H, V157D, Q165T, S230P, A253P, S359D | + |
| 1338 | V50K, Q165T, I227K, S359D, Q382A, N401D, E405P | + |
| 1339 | Q165P, Q169R, S230P, S359D | + |
| 1340 | V50D, Q151I, Q165T, S230P, S339E, S359D, Q382D, E405P, | + |
| | L436D, S437P | |
| 1341 | V92R, V157D, Q165T, I227K, S339E, S359D, P363D, V396E, | + |
| | E405P | |
| 1342 | V157H, Q165T, I227K, S339E, S359D | + |
| 1343 | V92S, V157D, Q165T, S230P, A253P, S339Q, Q382A, E445D | + |
| 1344 | V157S, Q165T, I227K, S339E, S359D, Q382D, S437P | + |
| 1345 | Q165P, Q169R, Q272H, S339Q, Q382A | + |
| 1346 | V50R, V164E, Q165T, I227K, S339E, S359D, Q382R | + |
| 1347 | V92R, Q165T, S359D | + |
| 1348 | V157S, Q165T, V252N, A253P, S339E, S359D | + |
| 1349 | V92S, L162I, Q165P, Q169R, S230P, S339E, S359D, Q382D, | + |
| | E405P, S437P | |
| 1350 | Q165P, Q169R, S230P, A253P, S339Q, Q382A | + |
| 1351 | Q8L, V50H, V157D, Q165T, P181A, I227K, S354G, S359D, | + |
| | S437P | |
| 1352 | V157S, Q165T, S230P, A253P, S336N, S359D | + |
| 1353 | V92S, V157S, Q165T, S230P, A253P, A259E, S339Q | + |
| 1354 | I95N, Q165T, I227A, S339Q, S359D | + |
| 1355 | V164E, Q165T, I227K, S339Q, Q382D, V396E, E405P, S437P | + |
| 1356 | Q165P, Q169R, I227K, M243I, S336N, S359D | + |
| 1357 | Q165P, Q169R, A176G, I227M, S359D | + |
| 1358* | P96S, T161S, Q165P, Q169R, I227K, S359D | + |
| 1359 | M163L, Q165P, Q169R, A212R, I227K, S336N, S359D | + |
| 1360 | T161N, Q165P, Q169R, I227K, S339E, S359D | + |
| 1361 | M163L, Q165P, Q169R, A212P, I227K, S336N, S359D | + |
| 1362 | M163L, Q165P, Q169R, I227K, S359D | + |
| 1363 | T161N, Q165P, Q169R, I227K, S359D | + |
| 1364 | V113I, I130V, Q165P, Q169R, I227K, S339E, S359D | + |
| 1365 | V113I, Q165P, Q169R, I227K, S359D | + |
| 1366 | S132I, Q165P, Q169R, I227K, S336N, S359D | + |
| 1367 | Q165P, Q169R, A212P, I227K, S336N, S359D | + |
| 1368 | R64C, L128H, Q165P, Q169R, A213G, I227K, S359D, A360V | + |
| 1369 | L128H, Q165P, Q169R, I227K, S359D, A360V | + |
| 1370 | Y120E, Q165T, Q169R, I227K, S359D | + |
| 1371 | R64C, S123Y, Q165P, Q169R, A176G, I227K, S359D | + |
| 1372 | L162I, Q165P, Q169R, I227K, S336N, S339E, S359D, Q382D | +++ |
| 1373 | Q165P, Q169R, I227K, S336N, S339E, S359D, Q382D | +++ |
| 1374 | Q165P, Q169R, I227M, S339E, L356E, S359D, Q382D | +++ |
| 1375 | Q165P, Q169R, I227K, S336N, S339E, N358E, S359D, Q382D | +++ |
| 1376 | L162I, Q165P, Q169R, I227T, S336N, S339E, N358E, S359D, | +++ |
| | Q382D | |
| 1377 | Q165P, Q169R, I227K, S339E, S359D, Q382D | +++ |
| 1378 | Q165P, Q169R, I227T, S336N, S339E, S359D, Q382D | ++ |
| 1379* | Q165P, Q169H, I227K, S339E, S359D, Q382D | ++ |
| 1380 | Q37H, Q165P, Q169R, I227K, S336N, S339E, S359D, Q382D | ++ |
| 1381 | Q165P, I227K, S339E, L356E, S359D, Q382D | ++ |
| 1382 | Q165P, Q169R, I227M, S336N, S339E, S359D, Q382D | ++ |
| 1383 | Q165P, Q169R, A176G, I227K, S339E, S359D, Q382D | ++ |
| 1384 | Q165P, Q169R, I227M, S336T, S339E, S359D, Q382D | ++ |
| 1385 | Q165P, I227K, S339E, S359D, Q382D | ++ |
| 1386 | Q165P, Q169R, I227K, S339E, L356E, S359D, Q382D | ++ |
| 1387 | L162I, Q165P, Q169R, I227K, G311D, S339E, N358E, S359D, | ++ |
| | Q382D | |
| 1388 | Q165P, Q169R, A176G, I227K, S339E, N358E, S359D, Q382D | + |
| 1389 | Q165P, Q169R, I227M, S339E, S359D, Q382D | + |
| 1390 | Q165P, Q169R, I227T, S336N, S339E, N358E, S359D, Q382D | + |
| 1391 | L128H, Q165P, Q169R, I227K, S339E, S359D, Q382D | + |
| 1392* | Q165P, I227K, S339E, S359D, Q382D | + |
| 1393* | Q165P, Q169R, I227K, S339E, L356E, S359D, Q382D | + |
| 1394 | Q37H, Q165P, Q169R, I227T, S339E, S359D, Q382D | + |
| 1395 | L128H, Q165P, Q169R, I227K, S260D, S339E, S359D, Q382D | + |
| 1396 | Q165P, Q169D, I227K, S339E, S359D, Q382D | + |
| 1397 | Y120N, Q165P, Q169R, I227K, S336N, S339E, S359D, Q382D | + |
| 1398 | Q165P, Q169R, K224E, I227M, S339E, S359D, Q382D | + |
| 1399 | Q165P, Q169R, A212P, I227K, S339E, N358E, S359D, Q382D | + |
| 1400 | G76D, Q165P, Q169R, I227K, S339E, S359D, Q382D | + |
| 1401 | S94N, Q165P, Q169R, I227K, S336N, S339E, S359D, Q382D | + |
| 1402 | Q165P, Q169R, I227K, S260D, S339E, S359D, Q382D | + |
| 1403 | G76D, L128H, Q165P, Q169R, A176G, I227K, S336N, S339E, | + |
| | S359D, Q382D | |
| 1404 | Q165P, Q169R, A176G, I227K, S336N, S339E, S359D, Q382D | + |
| 1405* | Q165P, Q169R, I227M, S339E, S359D, Q382D | + |
| 1406 | Q37H, Q165P, Q169R, I227K, S339E, S359D, Q382D | + |
| 1407 | G21K, N118D, Q165P, I227K, S339E, S359D, Q382D | + |
| 1408 | L162I, Q165P, Q169R, I227K, S339E, S359D, Q382D | + |
| 1409 | Y120N, Q165P, Q169R, I227K, S339E, S359D, Q382D | + |
| 1410 | Q165P, Q169R, A176G, I227K, S336N, S339E, S359D, A360D, | + |
| | Q382D | |
| 1411 | Q37H, L128H, Q165P, Q169R, I227K, S336N, S339E, S359D, | + |
| | Q382D | |
| 1412 | Q165P, Q169R, I227M, S336N, S339E, N358E, S359D, Q382D | + |
| 1413 | Q165P, Q169R, I227K, S339E, S359D, Q382D, G384S | + |
| 1414 | Q165P, Q169R, I227K, S260D, S339E, S359D, A360D, Q382D | + |
| 1415 | N118D, Q165P, Q169R, I227K, S339E, S359D, Q382D | + |
| 1416 | Q165P, Q169R, I227K, S339E, L356E, S359D, A360D, Q382D | + |
| 1417 | Q165P, Q169R, I227K, S260D, S336N, S339E, S359D, Q382D | + |
| 1418* | Q165P, Q169R, K224E, I227K, S339E, S359D, Q382D | + |
| 1419* | Q165P, Q169R, I227K, S339E, S359D, Q382D | + |

0 = 1.0 to 1.2 fold improvement over M. thermophila wild-type CBH2b.
++ = >1.2 to 1.4 fold improvement over M. thermophila wild-type CBH2b.
+++ >1.4 fold improvement over M. thermophila wild-type CBH2b.
*These variants further comprise at least one mutation in the signal peptide.

### Example 13

### Performance Sensitive Positions Identified in M. thermophila Cellobiohydrolases Type 2a and 2b that Improve Protein Activity and Stability

Beneficial mutations at amino acid positions in M. thermophila CBH2b that were identified as imparting improved cellobiohydrolase activity on biomass substrate (wheat straw pretreated under acidic conditions) were compared to beneficial mutations in M. thermophila CBH2a variants that were identified as having improved stability, in order to identify common positions for beneficial mutations in CBH2a and CBH2b. Beneficial amino acid substitutions from the wild-type residue which impart improved activity were determined for CBH2b as described in Example 12. Beneficial amino acid substitutions from the wild-type residue which impart improved stability were determined for CBH2a as described in Example 11. Corresponding amino acid positions between CBH2a and CBH2b were identified by aligning CBH2a and CBH2b using the protein alignment tool Protein Align as shown in FIG. 1 and FIG. 2. As shown in Table 7, sequence analysis of the CBH2b variants having improved activity on biomass and the CBH2a variants having improved stability showed that there were multiple positions at which beneficial amino acid substitutions were identified for both CBH2b and CBH2a.

TABLE 7
Amino acid positions common to CBH2b and CBH2a for mutations
that improve thermoactivity in CBH2b and thermostability in CBH2a

| CBH2b substitution | CBH2a substitution |
|---|---|
| V92DKRS | R11INTV |
| S94N | A13P |
| I95HN | S14L |
| P96ES | A15FIS |
| T161NS | R81K |
| A176GR | V95L |
| A213GHQ | D122S |
| N249DS | N159H |
| S336AELNT | S250CPV |
| N358E | K272A |
| S359DY | S273AD |
| G384S | G297M |
| A427T | A340S |
| Q448T | T361EKQ |

Of the amino acid positions identified in Table 7 as common positions for beneficial mutations imparting both improved activity in CBH2b and improved stability in CBH2a, four positions (S336, S359, G384, and Q448) were further identified as positions in which amino acid substitutions from the wild-type residue also impart improved stability on CBH2b, as shown in Table 5 above.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A recombinant cellobiohydrolase type 2b (CBH2b) variant comprising at least about 80% sequence identity to SEQ ID NO:1 and comprising an amino acid substitution, relative to SEQ ID NO:1, at one or more positions selected from 165, 169, and 359, wherein the position is numbered with reference to SEQ ID NO:1.

2. The variant of claim 1, wherein the amino acid residue at position 165 is proline, arginine, or threonine (X165P/R/T); the amino acid residue at position 169 is aspartic acid, lysine, leucine, or arginine (X169D/K/L/R); and/or the amino acid residue at position 359 is aspartic acid, lysine, or tyrosine (X359D/K/Y).

3. (canceled)

4. The variant of claim 1, further comprising an amino acid substitution, relative to SEQ ID NO:1, at one or more positions selected from 126, 128, 227, 339, and 360.

5. The variant of claim 4, wherein the amino acid residue at position 126 is glutamic acid, leucine, or methionine (X126E/L/M), the amino acid residue at position 128 is glutamic acid or histidine (X128E/H), the amino acid residue at position 227 is alanine, glycine, histidine, lysine, methionine, glutamine, or threonine (X227A/G/H/K/M/Q/T), the amino acid residue at position 339 is glutamic acid, leucine, glutamine, arginine, valine, or tryptophan (X339E/L/Q/R/V/W), and/or the amino acid residue at position 360 is cysteine, aspartic acid, glutamic acid, lysine, glutamine, arginine, serine, threonine, or valine (X360C/D/E/K/Q/R/S/T/V).

6. (canceled)

7. The variant of claim 1, further comprising an amino acid substitution, relative to SEQ ID NO:1, at one or more positions selected from 64, 86, 87, 102, 206, 212, 230, 253, 267, 271, 311, 332, 336, 340, 382, and 429.

8. The variant of claim 7, wherein the amino acid residue at position 64 is cysteine (X64C), the amino acid residue at position 86 is threonine (X86T), the amino acid residue at position 87 is threonine (X87T), the amino acid residue at position 102 is cysteine or tryptophan (X102C/W), the amino acid residue at position 206 is histidine or lysine (X206H/K), the amino acid residue at position 212 is cysteine, leucine, asparagine, proline, arginine, or serine (X212C/L/N/P/R/S), the amino acid residue at position 230 is proline (X230P), the amino acid residue at position 253 is asparagine, proline, or threonine (X253N/P/T), the amino acid residue at position 267 is glutamic acid, lysine, or leucine (X267E/K/L), the amino acid residue at position 271 is alanine (X271A), the amino acid residue at position 311 is aspartic acid or glutamine (X311D/Q), the amino acid residue at position 332 is serine (X332S), the amino acid residue at position 336 is alanine, glutamic acid, histidine, lysine, leucine, asparagine, proline, or threonine (X336A/E/H/K/L/N/P/T), the amino acid residue at position 340 is asparagine (X340N), the amino acid residue at position 382 is alanine, aspartic acid, histidine, or arginine (X382A/D/H/R), and/or the amino acid residue at position 429 is aspartic acid, histidine, or asparagine (X429D/H/N).

9. The variant of claim 1, wherein the variant is a Myceliophthora thermophila cellobiohydrolase.

10. The variant of claim 1 comprising at least 95% sequence identity to SEQ ID NO:1.

11. The variant of claim 1 that has an improved property relative to wild-type Myceliophthora thermophila CBH2b (SEQ ID NO:1).

12-15. (canceled)

16. A recombinant cellobiohydrolase type 2b (CBH2b) variant comprising at least about 80% sequence identity to SEQ ID NO:1 and comprising an amino acid substitution at one or more positions selected from A1, R7, C27, T73, A99, T100, S111, D119, Y120, Y121, H126, L128, Q151, Q165, S168, Q169, I227, S230, N245, M250, N251, A253, S260, V267, Q272, P276, H286, W289, W292, A294, N295, Q297, E301, G311, N325, N327, S333, A334, S336, S339, N341, F353, S359, A360, P363, Q381, Q382, G384, R397, G403, E405, D424, T425, S426, R429, Y432, L436, S437, Q441, Q448, T459, and P464, wherein the position is numbered with reference to SEQ ID NO:1.

17. (canceled)

18. The variant of claim 16, wherein the variant comprises one or more amino acid substitutions selected from A1V, R7S, C27Y, T73A, A99P, T100G/N, S111N, D119P/R, Y120H, Y121R, H126E, L128H, Q151L, Q165P/R, S168T, Q169K/L/R, 1227A/G/H/K/M/Q, S230P, N245T, M250G, N251D/T, A253P/T, S260K, V267E/K/L, Q272R, P276T, H286Q/S, W289C/M/S, W292A/H/P/R, A294R, N295R, Q297K/P/R/Y, E301K, G311Q, N325H, N327L, S333F, A334P, S336H/K/N/P/T, S339Q/R/W, N341V, F353I, S359D/K, A360C/K/T, P363D/H/V, Q381L, Q382R, G384T, R397H, G403T, E405G/P, D424N/Q, T425K/P/R, S426K, R429D/H/N, Y432W, L436K, S437G/P, Q441K, Q448K, T459G/K/N/R, and P464R.

19-36. (canceled)

37. A recombinant cellobiohydrolase type 2b (CBH2b) variant comprising at least about 80% sequence identity to SEQ ID NO:1 and comprising an amino acid substitution at one or more positions selected from P2, E6, R7, Q8, A12, W14, G18, N20, G21, A29, T33, A36, Q37, W40, N47, Q49, V50, P56, T61, R64, S67, R74, G76, S81, T83, P86, P87, V92, S94, I95, P96, A99, T100, S101, T102, S106, G107, G112, V113, A117, N118, Y120, S123, H126, L128, I130, S132, M133, A139, S142, A143, E146, Q151, V157, I159, D160, T161, L162, M163, V164, Q165, T166, S168, Q169, A176, A178, N179, P181, S206, N209, G210, A212, A213, K224, I227, E228, S230, M243, V247, T248, N249, V252, A253, S256, A259, S260, V267, K271, Q272, Q297, N308, G311, K312, A332, S336, S339, P340, N341, F353, S354, L356, N358, S359, A360, P363, A364, R365, Q382, G384, V396, A400, N401, H404, E405, A427, A428, L436, S437, E445, Q448, and T459, wherein the position is numbered with reference to SEQ ID NO:1.

38. (canceled)

39. The variant of claim 37, wherein the variant comprises one or more amino acid substitutions selected from P2H/S, E6N, R7H/S, Q8L/P, A12I, W14L, G18D, N20L/S, G21D/K, A29R/T, T33H, A36E, Q37F/H/L, W40L, N47K, Q49K, V50D/E/H/K/R, P56T, T61A, R64C, S67G, R74S, G76D, S81P, T83D, P86T, P87T, V92D/K/R/S, S94N, 195H/N, P96E/S, A99V, T100V, S101G, T102C/W, S106W/Y, G107D, G112E, V113I, A117T, N118D, Y120E/N/R, S123R/Y, H126E/L/M, L128E/H, I130V, S1321, M133F/V, A139H/T, S142E, A143M, E146L, Q151I/L, V157D/H/S, I159S, D160H, T161N/S, L162I, M163A/L, V164E/R, Q165P/T, T166R, S168G/Q/R, Q169D/R, A176G/R, A178N, N179D, P181A, S206H/K, N209S, G210A, A212C/L/N/P/R/S, A213G/H/Q, K224A/E/W, I227A/H/K/M/T, E228G, S230P, M243I, V247A, T248S, N249D/S, V252N, A253N/P/T, S256R, A259E, S260D/K, V267L, K271A, Q272H, Q297R, N308E, G311D, K312A, A332S, S336A/E/L/N/T, S339E/L/Q/V, P340N, N341D, F353L, S354G, L356E/G/H, N358E, S359D/Y, A360D/E/Q/R/S/T/V, P363D, A364T, R365G/L, Q382A/D/H/R, G384S, V396E/R, A400V, N401D, H404N, E405P/Q, A427T, A428N/S, L436D/N, S437P, E445D, Q448T, and T459R.

40-41. (canceled)

42. A recombinant cellobiohydrolase type 2b (CBH2b) variant comprising at least 50% sequence identity to SEQ ID NO:1 and comprising one or more amino acid substitutions selected from:
an aspartic acid, isoleucine, lysine, asparagine, arginine, serine, or threonine residue at position 92 (X92D/I/K/N/R/S/T);
an asparagine or proline residue at position 94 (X94N/P);
a histidine, leucine, or asparagine residue at position 95 (X95H/L/N);
a glutamic acid, phenylalanine, isoleucine, or serine residue at position 96 (X96E/F/I/S);
a cysteine or asparagine residue at position 111 (X111C/N);
an alanine, cysteine, lysine, proline, arginine, or valine residue at position 119 (X119A/C/K/P/R/V);
a lysine, asparagine, or serine residue at position 161 (X161K/N/S);
an alanine, leucine, or arginine residue at position 176 (X176G/L/R);
a glycine, histidine, glutamine, or serine residue at position 213 (X213G/H/Q/S);
an aspartic acid, histidine, or serine residue at position 249 (X249D/H/S);
a cysteine, glycine, leucine, or methionine residue at position 250 (X250C/G/L/M);
a cysteine, methionine, serine, or threonine residue at position 289 (X289C/M/S/T);
a glutamine, arginine, or tryptophan residue at position 294 (X294Q/R/W);
an alanine, cysteine, glutamic acid, histidine, lysine, leucine, asparagine, proline, threonine, or valine residue at position 336 (X336A/C/E/H/K/L/N/P/T/V);
an alanine or glutamic acid residue at position 358 (X358A/E);
an alanine, aspartic acid, lysine, or tyrosine residue at position 359 (X359A/D/K/Y);
a methionine, serine, or threonine residue at position 384 (X384M/S/T);
a serine or threonine residue at position 427 (X427S/T);
a glutamic acid, proline, or tryptophan residue at position 432 (X432E/P/W); and
a glutamic acid, lysine, glutamine, or threonine residue at position 448 (X448E/K/Q/T), wherein the position is numbered with reference to SEQ ID NO:1, and wherein the cellobiohydrolase variant has increased thermostability and/or thermoactivity in comparison to the wild-type cellobiohydrolase from which the variant is derived.

43-47. (canceled)

48. A recombinant polynucleotide encoding the variant of claim 1.

49. An expression vector comprising the recombinant polynucleotide of claim 48.

50. A recombinant host cell expressing a cellobiohydrolase variant having the amino acid sequence of a polypeptide of claim 1.

51. The recombinant host cell of claim 50, wherein the host cell is a yeast or filamentous fungus.

52. An enzyme composition comprising the variant cellobiohydrolase polypeptide of claim 1.

53. (canceled)

54. A method of producing a cellobiohydrolase type 2b (CBH2b) variant, the method comprising culturing the host cell of claim 50 under conditions sufficient for the expression of the cellobiohydrolase polypeptide by the cell.

55. (canceled)

56. A method of producing a fermentable sugar from a cellulosic substrate, comprising contacting the cellulosic substrate with a β-glucosidase (BGL), a type 2 endoglucanase (EG2), a type 1a cellobiohydrolase (CBH1a), a glycoside hydrolase 61 protein (GH61), and the CBH2b variant of claim 1 under conditions in which the fermentable sugar is produced.

57. The method of claim 56, comprising:
contacting the fermentable sugar with a microorganism in a fermentation to produce an end-product.

58. (canceled)

59. The method of claim 57, wherein the end-product is an alcohol, an amino acid, an organic acid, a diol, or glycerol.

60-65. (canceled)
